(19) United States
(12) Patent Application Publication
(10) Pub. No.: US 2026/0083504 A1
(43) Pub. Date: Mar. 26, 2026

(54) SURGICAL INSTRUMENT KINEMATICS PROCESSING, NAVIGATION, AND FEEDBACK

(71) Applicant: Intuitive Surgical Operations, Inc., Sunnyvale, CA (US)

(72) Inventors: Erez POSNER, Rehovot (IL); Moshe BOUHNIK, Holon (IL); Daniel DOBKIN, Tel-Aviv (IL); Netanel FRANK, Tel Aviv (IL); Liron LEIST, San Jose, CA (US); Emmanuelle MUHLETHALER, Tel-Aviv (IL); Roee SHIBOLET, Tel-Aviv (IL); Aniruddha TAMHANE, San Jose, CA (US); Adi ZHOLKOVER, Tel-Aviv (IL)

(21) Appl. No.: 19/112,395

(22) PCT Filed: Oct. 9, 2023
(86) PCT No.: PCT/US2023/034764

§ 371 Date: Mar. 17, 2025

Related U.S. Application Data
(60) Provisional application No. 63/415,220, filed on Oct. 11, 2022.
(60) Provisional application No. 63/415,225, filed on Oct. 11, 2022.
(60) Provisional application No. 63/415,231, filed on Oct. 11, 2022.

(51) Int. Cl. A61B 34/10; G06T 7/00; G06T 7/246; G06T 7/579; G06T 7/68; G06T 7/73; G06T 19/00

(52) U.S. Cl. CPC A61B 34/10; G06T 7/0014; G06T 7/248; G06T 7/579; G06T 7/68; G06T 7/74; G06T 19/003; A61B 2034/104; G06T 2207/10068; G06T 2207/20084; G06T 2207/30008; G06T 2207/30028; G06T 2210/41

(57) ABSTRACT

Various of the disclosed embodiments provide systems and methods for determining precise surgical instrument kinematics data, such as the pose of a colonoscope when examining a large intestine. The pose may then be used when constructing of a model of the patient interior from which reference geometries, such as a centerline, and navigational feedback, such as lacunae in the operator's review, may be produced. Some embodiments may also determine a data frame's suitability for downstream processing based upon the intraoperative field of view (e.g., downstream localization and mapping operations), such as whether the frame is undesirably blurred or depicts an obstruction. The operating surgeon, or reviewers of the surgical procedure, may then be presented, e.g., with metrics or graphical feedback related to one or more of: surgical instrument movement relative to the centerline, comprehensiveness of the operator's examination, and the amount of undesirable frames encountered during the examination.

### CROSS-REFERENCE TO RELATED APPLICATIONS

This application claims the benefit of, and priority to, U.S. Provisional Application No. 63/415,220, filed upon Oct. 11, 2022, entitled “SURGICAL INSTRUMENT REFERENCE KINEMATICS DETERMINATION AND DISPLAY”, U.S. Provisional Application No. 63/415,225, filed upon Oct. 11, 2022, entitled “INTRAOPERATIVE SENSOR VISUAL FIELD CHARACTERIZATION AND PROCESSING”, and U.S. Provisional Application No. 63/415,231, filed upon Oct. 11, 2022, entitled “COMPUTER STRUCTURES AND INTERFACES FOR INTRAOPERATIVE SURGICAL NAVIGATION”, each of which is incorporated by reference herein in its entirety for all purposes.

### TECHNICAL FIELD

Various of the disclosed embodiments relate to systems and methods for assessing surgical instrument kinematic behavior, e.g., for navigation, analysis, feedback, or for improved modeling of internal anatomical structures.

### BACKGROUND

While machine learning, network connectivity, surgical robotics and a variety of other new technologies hold great potential for improving healthcare efficacy and efficiency, many of these technologies and their applications cannot realize their full potential without accurate and consistent monitoring of instrument motion during a surgical procedure. Mechanical encoders and similar technologies may facilitate monitoring of surgical instrument positions, but they often are not consistent across surgical theaters and may not provide data readily comparable to systems with different or no such monitoring technology. In addition, mechanical solutions may require specific hardware and repairs, imposing additional costs and a reluctance to adequately maintain the system in a fully calibrated state. The inability to efficiently and economically acquire instrument kinematics data across disparate surgical theater configurations also makes it difficult to provide meaningful and consistent feedback to a surgeon, to analyze the surgeon's performance, and to provide comparisons between the surgeon's performance with that of other practitioners. Variance in acquisition methods across surgical theaters may bias the assessments of different surgeons and their surgical procedures, resulting in inaccurate, and possibly harmful, conclusions.

However, even such systems may be adversely affected when unsuitable data from the in-vivo sensor (e.g., where there is moisture upon the sensor, where sensor motion has blurred the field of view, where an occlusion prevents proper data gathering, etc.). Downstream processing which does not anticipate such corruption may itself become corrupted by these inputs, producing “garbage out” as a consequence of the “garbage in.” Even more unfortunately, in many downstream processing systems, particularly those which iteratively consider previously generated results in their input, such improper output may produce a cascade of improper results, causing even subsequently valid inputs to be improperly considered, as the process tries to reconcile the new, valid inputs, with the previously considered, invalid inputs. While there is value in simply excluding non-viable images, consistently recognizing when, where, and how often such unsuitable data instances are encountered may itself be informative of the surgeon's performance and the conditions of the surgical procedure.

Availability of such high quality kinematics results may then enable a variety of downstream applications. For example, even experienced surgeons may find it difficult to navigate and orient themselves within the patient interior. The pressures and distractions of the surgical theater, the wide variation in anatomical structures between patient populations, and the variation in surgical theater instrumentation and configurations can each easily precipitate confusion and disorientation. During a colonoscopy, for example, it can be difficult for the surgeon to know how much of the intestine remains to be examined, how much has been already examined, how thoroughly various regions of the intestine have been examined, how the examination compares to past or related examinations, etc. As staff shortages and an aging population continue to pressure hospitals to do more with fewer resources, there also exists a need for surgical team members with varying levels of experience to perform surgical operations quickly, efficiently, and consistently.

Consequently, there exists a need for computer systems and methods able to first identify surgical instrument fields of view suitable for downstream instrument kinematics processing, as well as systems and methods to assess the kinematic behavior of surgical instruments based upon those suitable fields of view, and, finally, systems and methods to assist surgical teams to consistently orient and navigate within patient interiors using such kinematics results.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation as may occur in relation to some embodiments;

FIG. 1B is a schematic view of various elements appearing in a surgical theater during a surgical operation employing a surgical robot as may occur in relation to some embodiments;

FIG. 2A is a schematic illustration of an organ, in this example a large intestine, with a cutaway view revealing the progress of a colonoscope during a surgical examination as may occur in connection with some embodiments;

FIG. 2B is a schematic illustration of a colonoscope distal tip as may be used in connection with some embodiments;

FIG. 2C is a schematic illustration of a portion of a colon with a cutaway view revealing a position of a colonoscope relative to a plurality of haustra;

FIG. 2D is a schematic representation of a camera-acquired visual image and a corresponding depth frame acquired from the perspective of the camera of the colonoscope depicted in FIG. 2C;

FIG. 2E is a pair of images depicting a grid-like pattern of orthogonal rows and columns in perspective, as captured from a colonoscope camera having a rectilinear view and a colonoscope camera having a fisheye view, each of which may be used in connection with some embodiments;

FIG. 3A is a schematic illustration of a computer-generated three-dimensional model of a large intestine with portions of the model highlighted, in a first perspective, as may occur in some embodiments;

FIG. 3B is a schematic illustration of the computer-generated three-dimensional model of FIG. 3A in a second perspective;

FIG. 3C is a schematic illustration of the computer-generated three-dimensional model of FIG. 3A in a third perspective;

FIGS. 4A-C are temporally successive schematic two-dimensional cross-section representations of a colonoscope progressing through a large intestine, as may occur in some embodiments;

FIGS. 4D-F are two dimensional schematic representations of depth frames generated from the corresponding fields of view depicted in FIGS. 4A-C, as may occur in some embodiments;

FIG. 4G is a schematic two-dimensional representation of a fusion operation between the depth frames of FIGS. 4D-F to create a consolidated representation, as may occur in some embodiments;

FIG. 5 is a flow diagram illustrating various operations in an example process for generating a computer model of at least a portion of an internal body structure, such as an organ, as may be implemented in some embodiments;

FIG. 6 is a flow diagram illustrating a pre-processing variation of the process of FIG. 5, as may be implemented in some embodiments;

FIG. 7 is an example processing pipeline for generating at least a portion of a three-dimensional model of a large intestine from a colonoscope data capture, as may be implemented in some embodiments;

FIG. 8 is an example processing pipeline depicting a pre-processing variation of the pipeline of FIG. 7, as may be implemented in some embodiments;

FIG. 9A is an example processing pipeline for determining a depth map and coarse local pose from colonoscope images using two distinct neural networks, as may be implemented in some embodiments;

FIG. 9B is an example processing pipeline for determining a depth map and coarse local pose from colonoscope images using a single neural network, as may be implemented in some embodiments;

FIG. 10A is a flow diagram illustrating various operations in a neural network training process as may be performed upon the networks of FIGS. 9A and 9B in some embodiments;

FIG. 10B is a bar plot depicting an exemplary set of training results for the process of FIG. 10A as may occur in connection with some embodiments;

FIG. 11A is a flow diagram illustrating various operations in a new fragment determination process as may be implemented in some embodiments;

FIG. 11B is a schematic side-view representation of an endoscope's successive fields of view as relates to a frustum overlap determination, as may occur in some embodiments;

FIG. 11C is a schematic temporal series of cross-sectional views depicting a colonoscope colliding with a sidewall of a colon and the resulting changes in the colonoscope camera's field of view, as may occur in connection with some embodiments;

FIG. 11D is a schematic representation of a collection of fragments corresponding to the collision of FIG. 11C, as may be generated in some embodiments;

FIG. 11E is a schematic network diagram illustrating various keyframe relations following graph network pose optimization operations, as may be occur in some embodiments;

FIG. 11F is a schematic diagram illustrating fragments with associated Truncated Signed Distance Function (TSDF) meshes relative to a full model TSDF mesh as may be generated in some embodiments;

FIG. 12A is a schematic cross-sectional view of a colonoscope within a portion of a colon, and the resulting colonoscope camera field of view, during advancing and withdrawing motions as may be occur in some embodiments;

FIG. 12B is a schematic three-dimensional model of a portion of a colon with a medial centerline axis reference geometry, as may be used in some embodiments;

FIG. 12C is a schematic side perspective view of a colonoscope camera in an advancing orientation relative to a centerline reference geometry, as may occur in some embodiments;

FIG. 12D is a schematic side perspective view of a colonoscope camera in a withdrawing orientation relative to a centerline reference geometry, as may occur in some embodiments;

FIG. 12E is a schematic side perspective view of a colonoscope camera in an off-centerline orientation relative to a centerline reference geometry, as may occur in some embodiments;

FIG. 12F is a schematic side perspective view of a colonoscope camera in a rotated orientation relative to a centerline reference geometry, as may occur in some embodiments;

FIG. 12G is a schematic side perspective view of a colonoscope camera in a rotated off-centerline orientation relative to a curved centerline reference geometry;

FIG. 12H is a schematic side perspective view of a succession of points along a centerline reference geometry;

FIG. 12I is a schematic perspective view of an orientation upon a centerline reference geometry with radial spatial contexts, as may occur in some embodiments;

FIG. 12J is a schematic cross-sectional view of a patient's pelvic region during a robotic surgical procedure as may occur in some embodiments;

FIG. 12K is a schematic depiction of a patient interior cavity with a cylindrical reference geometry;

FIG. 12L is a schematic depiction of a patient interior cavity with a spherical reference geometry;

FIG. 13A is a schematic three-dimensional model of a colon with depictions of advancing and withdrawing pathways;

FIG. 13B is a flow diagram illustrating various operations in an example medial centerline estimation process as may be implemented in some embodiments;

FIG. 13C is a schematic three-dimensional model of a colon with an associated preexisting global centerline and local centerline for a segment, as may occur in some embodiments;

FIG. 13D is a flow diagram illustrating various operations in an example process for estimating a local centerline segment, as may be implemented in some embodiments;

FIG. 13E is a flow diagram illustrating various operations in an example process for extending a global centerline with a segment's local centerline, as may be implemented in some embodiments;

FIG. 14 is a schematic operational pipeline depicting various steps in an example process for updating a global medial axis centerline with local segment centerlines, as may be implemented in some embodiments;

FIG. 15A is a flow diagram illustrating various operations in an example process for updating instrument kinematics data relative to a reference geometry during a surgical procedure, as may be implemented in some embodiments;

FIG. 15B is a flow diagram illustrating various operations in an example process for assessing kinematics data, as may be implemented in some embodiments;

FIG. 15C is a schematic representation of a model with spatial and temporal contextual regions, as may be used in some embodiments;

FIG. 15D is a collection of graphical user interface (GUI) elements, as may be implemented in some embodiments;

FIG. 16A is a schematic view of a three-dimensional colon model with a path graphic, as may be presented in some embodiments;

FIG. 16B is a schematic view of a three-dimensional colon model with a medial centerline graphic and corresponding speed plot, as may be presented in some embodiments;

FIG. 16C is the schematic view of a path graphic in a three-dimensional cavity model as may be presented in some embodiments;

FIG. 16D is a schematic view of a three-dimensional cavity model with a spherical reference geometry graphic, as may be presented in some embodiments;

FIG. 16E is a schematic image from a centerline reference geometry kinematics icon animation, as may be presented in some embodiments;

FIG. 16F is a schematic image from a spherical reference geometry kinematics icon animation, as may be presented in some embodiments;

FIG. 17A is a collection of schematic GUI elements, as may be implemented in some embodiments;

FIG. 17B is a three-dimensional colon model GUI element and a speed plot GUI element as may be presented to a user in some embodiments;

FIG. 17C is an enlarged view of the reference geometry kinematics graphic depicted in the GUI of 17A;

FIG. 17D is a collection of schematic GUI elements appearing in a robotic surgical procedure interface, as may be implemented in some embodiments;

FIG. 17E is a collection of schematic GUI elements appearing in a robotic surgical procedure interface, as may be implemented in some embodiments;

FIG. 17F is an enlarged view of a guidance graphic depicted in the GUI elements of FIG. 17E;

FIG. 18A is a schematic collection of a various surgical camera states and the corresponding fields of view, as may occur in some embodiments;

FIG. 18B is a schematic cross-section of a patient's pelvic region during a laparoscopic procedure, as may occur in some embodiments;

FIG. 18C is a schematic perspective view of a surgical tool occluding a portion of a surgical camera's field of view, as may occur in some embodiments;

FIG. 18D is a schematic collection of a various surgical fields of view with a graphical interface overlay, as may occur in some embodiments;

FIG. 19A is a flow diagram illustrating various operations in a high-level classification process, as may be implemented in some embodiments;

FIG. 19B is a flow diagram illustrating various operations in an image pre-processing process, as may be implemented in some embodiments;

FIG. 19C is a flow diagram illustrating various operations in an image post-classification process, as may be implemented in some embodiments;

FIG. 20A is a schematic neural network architecture diagram of an example neural network structure as may be used in some embodiments;

FIG. 20B is a partial code listing for creating an example implementation of the network topology depicted in FIG. 20A;

FIG. 20C is a partial code listing for performing forward propagation upon the example network implementation of FIG. 20B;

FIG. 21A is a flow diagram illustrating various operations in a neural network training and validation process, as may be implemented in some embodiments;

FIG. 21B is a flow diagram illustrating various operations in a single or multi-classifier classification process, as may be implemented in some embodiments;

FIG. 22 is a flow diagram illustrating various operations in an example process for inferring the character of a surgical performance from classification results, as may be implemented in some embodiments;

FIG. 23A is a collection of schematic graphical elements as may appear in a GUI in some embodiments, including a visual image frame with a graphical interface overlay indicating a recent classification quality assessment, as may be implemented in some embodiments;

FIG. 23B is a schematic GUI element for providing intraoperative image quality feedback, as may be implemented in some embodiments;

FIG. 23C is a pair of successive GUI states during a surgical procedure with a schematic indication of intraoperative image quality feedback, as may be implemented in some embodiments;

FIG. 23D is a flow diagram illustrating various operations in an example process for providing user feedback regarding non-viable images, as may be implemented in some embodiments;

FIG. 23E is a schematic illustration of a surgical tool occluding a portion of a surgical camera's field of view, as may occur in some embodiments;

FIG. 24A is a collection of schematic GUI elements for reviewing a surgical procedure, as may be implemented in some embodiments;

FIG. 24B is a flow diagram illustrating various operations in an example process for responding to a user playback position selection, as may be implemented in some embodiments;

FIG. 24C is a flow diagram illustrating various operations in an example process for responding to a user model position selection, as may be implemented in some embodiments;

FIG. 25 is a schematic sequence of states for model, view, and projected mapping regions of a GUI in a coverage assessment process, as may be implemented in some embodiments;

FIG. 26A is an enlarged perspective of the model state at time 2500b in FIG. 25;

FIG. 26B is an enlarged perspective of the projected mapping state at time 2500b in FIG. 25;

FIG. 26C is a schematic representation of a pair of relatively rotated surgical camera orientations and their corresponding views;

FIG. 27 is a flow diagram illustrating various operations in an example process for performing the coverage assessment process of FIG. 25;

FIG. 28 is a schematic sequence of states for model, view, and projected mapping regions of FIG. 25, but with additional graphical guides, as may be implemented in some embodiments;

FIG. 29A is a collection of pairs of schematic and projected mapping regions for a local compass scope, as well as a corresponding schematic perspective view of the colon model, as may be implemented in some embodiments;

FIG. 29B is a projected mapping GUI element with a level-of-detail magnification, as may be implemented in some embodiments;

FIG. 30A is a schematic representation of a continuous navigation compass as may be implemented in some embodiments;

FIG. 30B is a schematic representation of a discontinuous navigation compass as may be implemented in some embodiments;

FIG. 30C is a schematic representation of a series of states in determining a relative position for displaying a highlight, e.g., upon the compass of FIG. 30A or FIG. 30B, as may occur in some embodiments;

FIG. 30D is a schematic representation of a projected image map region with columnar color references, as may be implemented in some embodiments;

FIG. 30E is a schematic perspective view of an augmented reality representation of a navigation guide from the perspective of a second camera, as may occur in some embodiment;

FIG. 31 is a flow diagram illustrating various operations in an example process for rendering the graphical guides of FIG. 28;

FIG. 32 is a schematic block diagram illustrating various components and their relations in an example processing pipeline for iterative internal structure representation and navigation, as may be implemented in some embodiments;

FIG. 33A is a schematic block diagram illustrating various operational relations between components of an example surface parametrization process as may be implemented in some embodiments;

FIG. 33B is a schematic block diagram illustrating various operational relations between components of an example surface flattening image update process as may be implemented in some embodiments;

FIG. 33C is a schematic block diagram illustrating various operational relations between components of an example navigation compass update process as may be implemented in some embodiments;

FIG. 34A depicts schematic representations of various GUI panels as they may be presented to a reviewer during a first point of time in a surgical procedure in some embodiments;

FIG. 34B depicts the schematic representations of various GUI panels in FIG. 34A, as they may be presented to a reviewer during a second point of time in a surgical procedure in some embodiments;

FIG. 34C is a perspective view of cylindrical reference geometry mesh and an example intestinal mesh geometry, as may be used in some embodiments;

FIG. 34D is a perspective view of spherical reference geometry mesh and an example cavity mesh geometry, as may be used in some embodiments;

FIG. 34E is a perspective view of cumulative convex hull reference geometry mesh and an example cavity mesh geometry, as may be used in some embodiments;

FIG. 34F is a schematic side-view of example correspondences between a portion of a reference geometry mesh and a portion of an intra-surgically derived mesh geometry, as may be used in some embodiments;

FIG. 35A is a schematic collection of GUI elements in an example colonoscope examination as may be presented to a reviewer in some embodiments;

FIG. 35B is a schematic collection of GUI elements in an example surgical robotic examination as may be presented to a reviewer in some embodiments;

FIG. 36A is a schematic representation of an incomplete model, contour determination, centerline guide path, and navigation compass, as may be implemented in some embodiments;

FIG. 36B is a collection of schematic perspective views of various orientation axes relative to a centerline during an example compass alignment process, as may be implemented in some embodiments;

FIG. 37 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

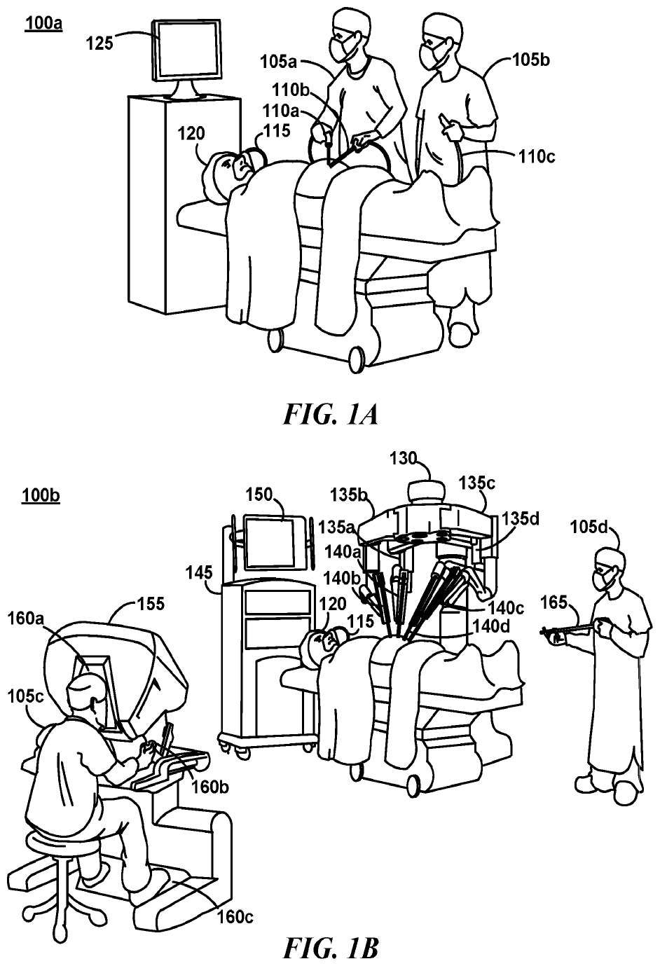

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc. and a mechanical end effector 110a such as scissors, retractors, a dissector, etc.

The visualization tool 110b provides the surgeon 105a with an interior view of the patient 120, e.g., by displaying visualization output from a camera mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply various of the two-dimensional operations discussed herein, mutatis mutandis, to such three-dimensional depth data when such data is available.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of end effectors, etc. throughout the surgery. In some embodiments, the data may have been recorded using an in-theater recording device, such as an Intuitive Data Recorder™ (IDR), which may capture and store sensor data locally or at a networked location.

### Example Organ Data Capture Overview

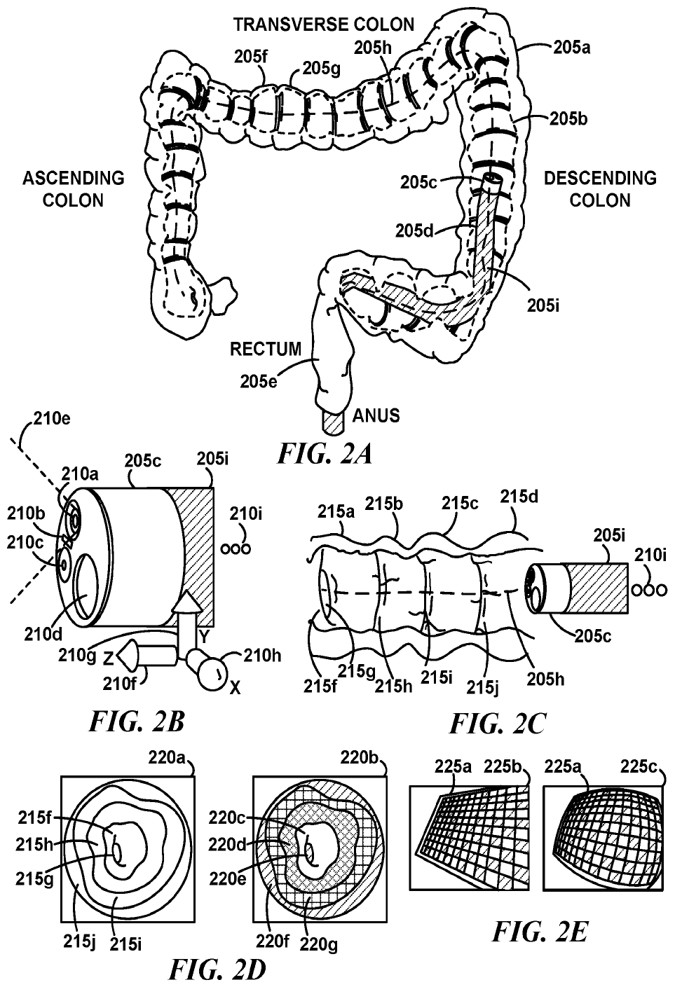

Whether in non-robotic surgical theater 100a or in robotic surgical theater 100b, there may be situations where surgeon 105a, assisting member 105b, the operator 105c, assisting member 105d, etc. seek to examine an organ or other internal body structure of the patient 120 (e.g., using visualization tool 110b or 140d). For example, as shown in FIG. 2A and revealed via cutaway 205b, a colonoscope 205d may be used to examine a large intestine 205a. While this detailed description will use the large intestine and colonoscope as concrete examples with which to facilitate the reader's comprehension, one will readily appreciate that the disclosed embodiments need not be limited to large intestines and colonoscopes, and indeed are here explicitly not contemplated as being so limited. Rather, one will appreciate that the disclosed embodiments may likewise be applied in conjunction with other organs and internal structures, such as lungs, hearts, stomachs, arteries, veins, urethras, regions between organs and tissues, etc. and with other instruments, such as laparoscopes, thorascopes, sensor-bearing catheters, bronchoscopes, ultrasound probes, miniature robots (e.g., swallowed sensor platforms), etc. Many such organs and internal structures will include folds, outcrops, and other structures, which may occlude portions of the organ or internal structure from one or more perspectives. For example, the large intestine 205a shown here includes a series of pouches known as haustra, including haustrum 205f and haustrum 205g. Thoroughly examining the large intestine despite occlusions in the field of view precipitated by these haustra and various other challenges, including possible limitations of the visualization tool itself, may be very difficult for the surgeon or automated system.

In the depicted example, the colonoscope 205d may navigate through the large intestine by adjusting bending section 205i as the operator, or automated system, slides colonoscope 205d forward. Bending section 205i may likewise be adjusted so as to orient a distal tip 205c in a desired orientation. As the colonoscope proceeds through the large intestine 205a, possibly all the way from the descending colon, to the transverse colon, and then to the ascending colon, actuators in the bending section 205i may be used to direct the distal tip 205c along a centerline 205h of the intestines. Centerline 205h is a path along points substantially equidistant from the interior surfaces of the large intestine along the large intestine's length. Prioritizing the motion of colonoscope 205d along centerline 205h may reduce the risk of colliding with an intestinal wall, which may harm or cause discomfort to the patient 120. While the colonoscope 205d is shown here entering via the rectum 205e, one will appreciate that laparoscopic incisions and other routes may also be used to access the large intestine, as well as other organs and internal body structures of patient 120.

FIG. 2B provides a closer view of the distal tip 205c of colonoscope 205d. This example tip 205c includes a visual image camera 210a (which may capture, e.g., color or grayscale images), light source 210c, irrigation outlet 210b, and instrument bay 210d (which may house, e.g., a cauterizing tool, scissors, forceps, etc.), though one will readily appreciate variations in the distal tip design. For clarity, and as indicated by the ellipsis 210i, one will appreciate that the bending section 205i may extend a considerable distance behind the distal tip 205c.

As previously mentioned, as colonoscope 205d advances and retreats through the intestine, joints, or other bendable actuators within bending section 205i, may facilitate movement of the distal tip 205c in a variety of directions. For example, with reference to the arrows 210f, 210g, 210h, the operator, or an automated system, may generally advance the colonoscope tip 205c in the Z direction represented by arrow 210f. Actuators in bendable portion 205i may allow the distal end 205c to rotate around the Y axis or X axis (perhaps simultaneously), represented by arrows 210g and 210h respectively (thus analogous to yaw and pitch, respectively). In this manner, camera 210a's field of view 210e may be adjusted to facilitate examination of structures other than those appearing directly before the colonoscope's direction of motion, such as regions obscured by the haustral folds.

Specifically, FIG. 2C is a schematic illustration of a portion of a large intestine with a cutaway view revealing a position of the colonoscope tip 205c relative to a plurality of haustral annular ridges. Between each of haustra 215a, 215b, 215c, 215d may lie an interstitial tissue forming an annular ridge. In this example, annular ridge 215h is formed between haustra 215a, 215b, annular ridge 215i is formed between haustra 215b, 215c, and annular ridge 215j is formed between haustra 215c, 215d. While the operator may wish the colonoscope to generally travel a path down the centerline 205h of the colon, so as to minimize discomfort to the patient, the operator may also wish for bendable portion 205i to reorient the distal tip 205c such that the camera 210a's field of view 210e may observe portions of the colon occluded by the annular ridges.

Regions further from the light source 210c may appear darker to camera 210a than regions closer to the light source 210c. Thus, the annular ridge 215j may appear more luminous in the camera's field of view than opposing wall 215f, and aperture 215g may appear very, or entirely, dark to the camera 210a. In some embodiments, the distal tip 205c may include a depth sensor, e.g., in instrument bay 210d. Such a sensor may determine depth using, e.g., time-of-flight photon reflectance data, sonography, a stereoscopic pair of visual image cameras (e.g., on extra camera in addition to camera 210a) etc. However, various embodiments disclosed herein contemplate estimating depth data based upon the visual images of the single visual image camera 210a upon the distal tip 205c. For example, a neural network may be trained to recognize distance values corresponding to images from the camera 210a (e.g., as variations in surface structures and the luminosity resulting from reflected light of light 210c at varying distance may provide sufficient correlations with depth between successive images for a machine learning system to make a depth prediction). Some embodiments may employ a six degree of freedom guidance sensor (e.g., the 3D Guidance® sensors provided by Northern Digital Inc.) in lieu of the pose estimation methods described herein, or in combination with those methods, such that the methods described herein and the six degree of freedom sensors provide complementary confirmation of one another's results.

Thus, for clarity, FIG. 2D depicts a visual image and a corresponding schematic representation of a depth frame acquired from the perspective of the camera of colonoscope depicted in FIG. 2C. Here, annular ridge 215j occludes a portion of annular ridge 215i, which itself occludes a portion of annular ridge 215h, while annular ridge 215h occludes a portion of the wall 215f. While the aperture 215g is within the camera's field of view, the aperture is sufficiently distant from the light source that it may appear entirely dark.

With the aid of a depth sensor, or via image processing of image 220a (and possibly a preceding or succeeding image following the colonoscope's movement) using systems and methods discussed herein, etc., a corresponding depth frame 220b may be generated, which corresponds to the same field of view producing visual image 220a. As shown in this example, the depth frame 220b assigns a depth value to some or all of the pixel locations in image 220a (though one will appreciate that the visual image and depth frame will not always have values directly mapping pixels to depth values, e.g., where the depth frame is of smaller dimensions than the visual image). One will appreciate that the depth frame, comprising a range of depth values, may itself be presented as a grayscale image in some embodiments (e.g., the largest depth value mapped to value of 0, the shortest depth value mapped to 255, and the resulting mapped values presented as a grayscale image). Thus, the annular ridge 215j may be associated with a closest set of depth values 220f, the annular ridge 215i may be associated with a further set of depth values 220g, the annular ridge 215h may be associated with a yet further set of depth values 220d, the back wall 215f may be associated with a distant set of depth values 220c, and the aperture 215g may be beyond the depth sensing range (or entirely black, beyond the light source's range) leading to the largest depth values 220e (e.g., a value corresponding to infinite, or unknown, depth). While a single pattern is shown for each annular ridge in this schematic figure to facilitate comprehension by the reader, one will appreciate that the annular ridges will rarely present a flat surface in the X-Y plane (per arrows 210h and 210g) of the distal tip. Consequently many of depth values within, e.g., set 220f, are unlikely to be the exact same value.

While visual image camera 210a may capture rectilinear images one will appreciate that lenses, post-processing, etc. may be applied in some embodiments such that images captured from camera 210a are other than rectilinear. For example, FIG. 2E is a pair of images 225b, 225c depicting a grid-like checkered pattern 225a of orthogonal rows and columns in perspective, as captured from a colonoscope camera having a rectilinear view and a colonoscope camera having a fisheye view, respectively. Such a checkered pattern may facilitate determination of a given camera's intrinsic parameters. One will appreciate that the rectilinear view may be achieved by undistorting the fisheye view, once the intrinsic parameters of the camera are known (which may be useful, e.g., to normalize disparate sensor systems to a similar form recognized by a machine learning architecture). A fisheye view may allow the user to readily perceive a wider field of view than in the case of the rectilinear perspective. As the focal point of the fisheye lens, and other details of the colonoscope, such as the light 210c luminosity, may vary between devices and even across the same device over time, it may be necessary to recalibrate various processing methods for the particular device at issue (consider the device's “intrinsics”, e.g., such as focal-length, principal points, distortion coefficients etc.) or to at least anticipate device variation when training and configuring a system.

### Example Computer Generated Organ Model

During, or following, an examination of an internal body structure (such as large intestine 205a) with a camera system (e.g., camera 210a), it may be desirable to generate a corresponding three-dimensional model of the organ or examined cavity. For example, various of the disclosed embodiments may generate a Truncated Signed Distance Function (TSDF) volume model, such as the TSDF model 305 of the large intestine 205a, based upon the depth data captured during the examination. While TSDF is offered here as an example to facilitate the reader's comprehension, one will appreciate a number of suitable three-dimensional data formats. For example, a TSDF formatted model may be readily converted to a vertex mesh, or other desired model format, and so references to a “model” herein may be understood as referring to any such format. Accordingly, the model may be textured with images captured via camera 210a or may, e.g., be colored with a vertex shader. For example, where the colonoscope traveled inside the large intestine, the model may include an inner and outer surface, the inner rendered with the textures captured during the examination and the outer surface shaded with vertex colorings. In some embodiments, only the inner surface may be rendered, or only a portion of the outer surface may be rendered, so that the reviewer may readily examine the organ interior.

Such a computer-generated model may be useful for a variety of purposes. For example, portions of the model may be differently textured, highlighted via an outline (e.g., the region's contour from the perspective of the viewer being projected upon the texture of a billboard vertex mesh surface in front of the model), called out with three dimensional markers, or otherwise identified, which are associated with, e.g.: portions of the examination bookmarked by the operator, portions of the organ found to have received inadequate review as determined by various embodiments disclosed herein, organ structures of interest (such as polyps, tumors, abscesses, etc.), etc. For example, portions 310a and 310b of the model may be vertex shaded, or outlined, in a color different or otherwise distinct from the rest of the model 305, to call attention to inadequate review by the operator, e.g., where the operator failed to acquire a complete image capture of the organ region, moved too quickly through the region, acquired only a blurred image of the region, viewed the region while it was obscured by smoke, etc. Though a complete model of the organ is shown in this example, one will appreciate that an incomplete model may likewise be generated, e.g., in real-time during the examination, following an incomplete examination, etc. In some embodiments, the model may be a non-rigid 3D reconstruction (e.g., incorporating a physics model to represent the behavior of tissues with varying stiffness).

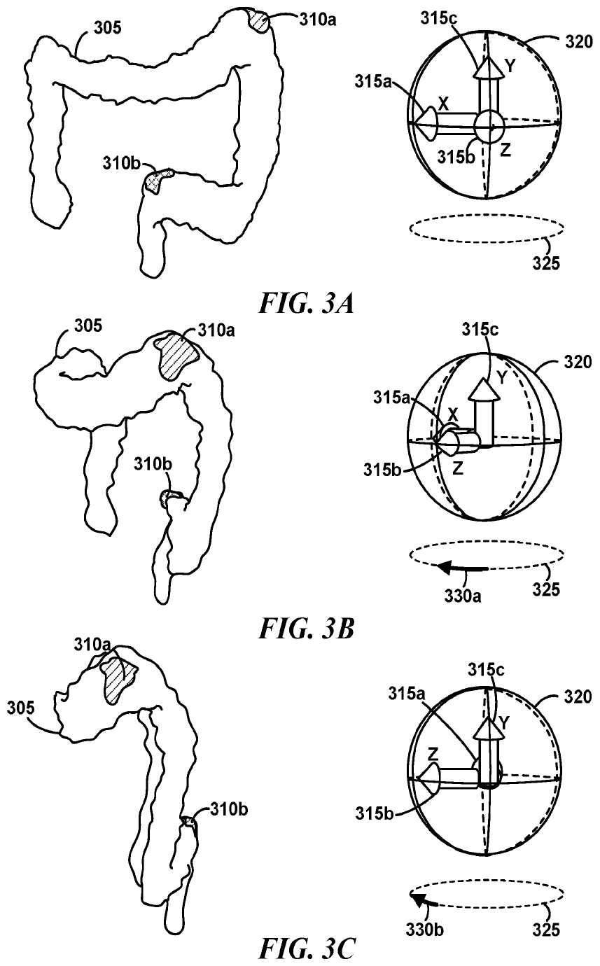

For clarity, each of FIGS. 3A, 3B, 3C depict the three-dimensional model 305 from a different perspective. Specifically, a coordinate reference 320, having X-Y-Z axes represented by arrows 315a, 315c, 315b respectively, is provided for the reader's reference. If the model were rendered about coordinate reference 320 at the model's center, then FIG. 3B shows the model 305 rotated approximately 40 degrees 330a around the Y-axis, i.e., in the X-Z plane 325, relative to the model 305's orientation in FIG. 3A. Similarly, FIG. 3C depicts the model 305 further rotated approximately an additional 40 degrees 330b to an orientation at nearly a right angle to that of the orientation in FIG. 3A. One will appreciate that the model 305 may be rendered only from the interior of the organ (e.g., where the colonoscope appeared), only the exterior, or both the interior and exterior (e.g., using two, complementary texture meshes). Where the only data available is for the interior of the organ, the exterior texture may be vertex shaded, textured with a synthetic texture approximating that of the actual organ, simply transparent, etc. In some embodiments, only the exterior is rendered with vertex shading. As discussed herein, a reviewer may be able to rotate the model in a manner analogous to FIGS. 3A, 3B, 3C, as well as translate, zoom, etc. so as, e.g., to more closely investigate identified regions 310a, 310b, to plan follow-up surgeries, to assess the organ's relation to a contemplated implant (e.g., a surgical mesh, fiducial marker, etc.), etc.

### Example Frame Generation and Consolidation Operations

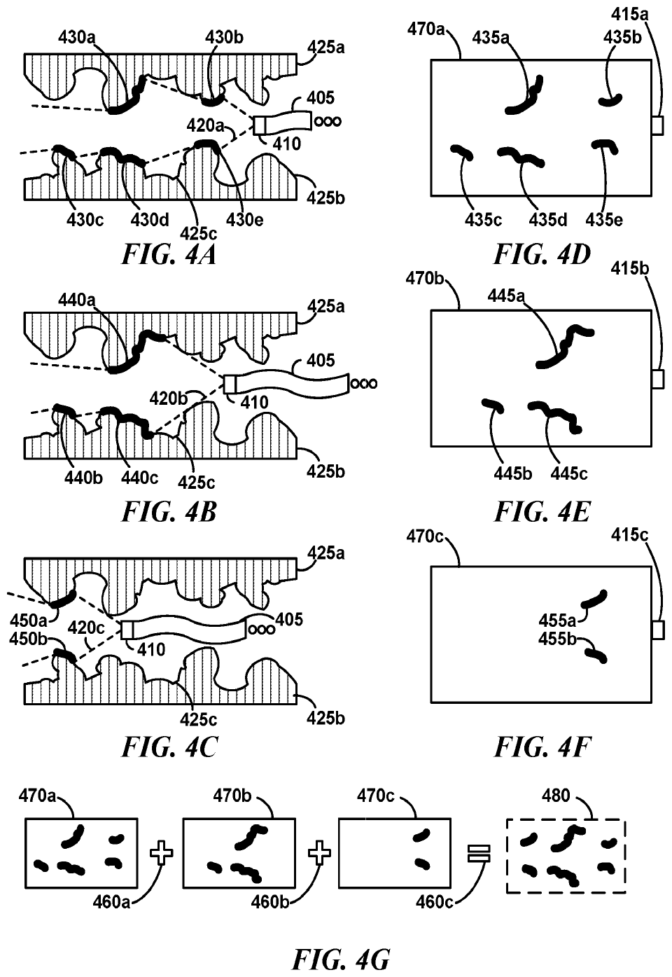

As depth data may be incrementally acquired throughout the examination, the data may be consolidated to facilitate creation of a corresponding three-dimensional model (such as model 305) of all or a portion of the internal body structure. For example, FIGS. 4A-C present temporally successive schematic two-dimensional cross-sectional representations of a colonoscope field of view, corresponding to the actual three-dimensional field of view, as the colonoscope proceeds through a colon.

Specifically, FIG. 4A depicts a two-dimensional cross sectional view of the interior of a colon, represented by top portion 425a and bottom portion 425b. As discussed, the colon interior, like many body interiors, may contain various irregular surfaces, e.g., where haustra are joined, where polyps form, etc. Accordingly, when the colonoscope 405 is in the position of FIG. 4A the camera coupled with distal tip 410 may have an initial field of view 420a. As the irregular surface may occlude portions of the colon interior, only certain surfaces, specifically the surfaces 430a, 430b, 430c, 430d, and 430e may be visible to the camera (and/or depth sensor) from this position. Again, as this is a cross sectional view similar to FIG. 2C, one will appreciate that such surfaces may correspond to the annular ridge surfaces appearing in the image 220a. That is, while surfaces are represented here by lines, one will appreciate that these surfaces may correspond to three dimensional structures, e.g., to the annular ridges between haustra, such as the annular ridges 215h, 215i, 215j. As a result of the limited field of view, a surgeon may have not yet viewed an occluded region, such as the region 425c outside the field of view 420a. One will appreciate that such limitations upon the field of view may be present whether the camera image is rectilinear, fisheye, etc.

As the colonoscope 405 advances further into the colon (from right to left in this depiction) as shown in FIG. 4B the camera's field of view 420b may now perceive surfaces 440a, 440b, and 440c. Naturally, portions of these surfaces may coincide with previously viewed portions of surfaces, as in the case of surfaces 430a and 440a. If the colonoscope's field of view continues to advance linearly, without adjustment (e.g., rotation of the distal tip via the bendable section 205i), portions of the occluded surface may remain unviewed. Here, e.g., the region 425c has still not appeared within the camera's field of view 420b despite the colonoscope's advancement. Similarly, as the colonoscope 405 advances to the position of FIG. 4C, surfaces 450a and 450b may now be visible in field of view 420c, but, unfortunately, the colonoscope will continue to have passed the region 425c without the region 425c appearing in the field of view.

One will appreciate that throughout colonoscope 405's progress, depth values corresponding to the interior structures before the colonoscope may be generated either in real-time during the examination or by post-processing of captured data after the examination. For example, where the distal tip 205c does not include a sensor specifically designed for depth data acquisition, the system may instead use the images from the camera to infer depth values (an operation which may occur in real-time or near real-time using the methods described herein). Various methods exist for determining depth values from images including, e.g., using a neural network trained to convert visual image data to depth values. For example, one will appreciate that self-supervised approaches for producing a network inferring depth from monocular images may be used, such as that found in the paper “Digging Into Self-Supervised Monocular Depth Estimation” appearing as arXiv™ preprint arXiv™:1806.01260v4 and by Clément Godard, Oisin Mac Aodha, Michael Firman, and Gabriel Brostow, and as implemented in the Monodepth2 self-supervised model described in that paper. However, such methods do not specifically anticipate the unique challenges present in this endoscopic context and may be modified as described herein. Where the distal tip 205c does include a depth sensor, or where stereoscopic visual images are available, the depth values from the various sources may be corroborated by the values from the monocular image approach.

Thus, a plurality of depth values may be generated for each position of the colonoscope at which data was captured to produce a corresponding depth data “frame.” Here, the data in FIG. 4A may produce the depth frame 470a of FIG. 4D, the data in FIG. 4B may produce the depth frame 470b of FIG. 4E, and the data in FIG. 4C may produce the depth frame 470c of FIG. 4F. Thus, depth values 435a, 435b, 435c, 435d, and 435e, may correspond to surfaces 430a, 430b, 430c, 430d, and 430e respectively. Similarly, depth values 445a, 445b, and 445c may correspond to surfaces 440a, 440b, and 440c, respectively, and depth values 455a and 455b may correspond to surfaces 450a and 450b.

Note that each depth frame 470a, 470b, 470c is acquired from the perspective of the distal tip 410, which may serve as the origin 415a, 415b, 415c for the geometry of each respective frame. Thus, each of the frames 470a, 470b, 470c may be considered relative to the pose (e.g., position and orientation as represented by matrices or quaternions) of the distal tip at the time of data capture and globally reoriented if the depth data in the resulting frames is to be consolidated, e.g., to form a three-dimensional representation of the organ as a whole (such as model 305). This process, known as stitching or fusion, is shown schematically in FIG. 4G wherein the depth frames 470a, 470b, 470c are combined 460a, 460b to form 460c a consolidated frame 480. Example methods for stitching together frames are described herein.

### Example Data Processing Operations

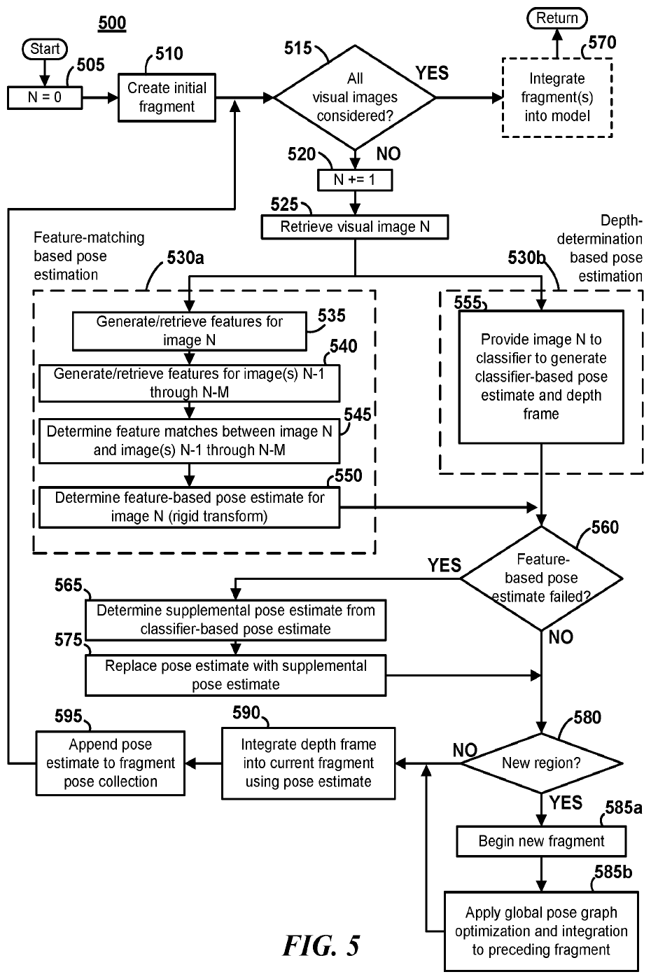

FIG. 5 is a flow diagram illustrating various operations in an example process 500 for generating a computer model of at least a portion of an internal body structure, as may be implemented in some embodiments. At block 505, the system may initialize a counter N to 0 (one will appreciate that the flow diagram is merely exemplary and selected to facilitate the reader's understanding, consequently, many embodiments may not employ such a counter or the specific operations disclosed in FIG. 5). At block 510 the computer system may allocate storage for an initial fragment data structure. As explained in greater detail herein, a fragment is a data structure comprising one or more depth frames, facilitating creation of all or a portion of a model. In some embodiments, the fragment may contain data relevant to a sequence of consecutive frames depicting a similar region of the internal body structure and may share a large intersection area over that region. Thus, a fragment data structure may include memory allocated to receive RGB visual images, visual feature correspondences between visual images, depth frames, relative poses between the frames within the fragment, timestamps, etc. At blocks 515 and 520 the system may then iterate over each image in the captured video, incrementing the counter accordingly, and then retrieving the corresponding next successive visual image of the video at block 525.

As shown in this example, the visual image retrieved at block 525 may then be processed by two distinct subprocesses, a feature-matching based pose estimation subprocess 530a and a depth-determination based pose estimation subprocess 530b, in parallel. Naturally, however, one will appreciate that the subprocesses may instead be performed sequentially. Similarly, one will appreciate that parallel processing need not imply two distinct processing systems, as a single system may be used for parallel processing with, e.g., two distinct threads (as when the same processing resources are shared between two threads), etc.

Feature-matching based pose estimation subprocess 530a determines a local pose from an image using correspondences between the image's features (such as Scale-Invariant Feature Transforms (SIFT) features) and such features as they appear in previous images. For example, one may use the approach specified in the paper “BundleFusion: Real-time Globally Consistent 3D Reconstruction” appearing as arXiv™ preprint arXiv™:1604.01093v3 and by Angela Dai, Matthias Niessner, Michael Zollhofer, Shahram Izadi, and Christian Theobalt, specifically, the feature correspondence for global Pose Alignment described in section 4.1 of that paper, wherein the Kabsch algorithm is used for alignment, though one will appreciate that the exact methodology specified therein need not be used in every embodiment disclosed here (e.g., one will appreciate that a variety of alternative correspondence algorithms suitable for feature comparisons may be used). Rather, at block 535, any image features may be generated from the visual image which are suitable for pose recognition relative to the previously considered images' features. To this end, one may use SIFT features (as in the “BundleFusion” paper referenced above), Speeded-Up Robust Features (SURF), Features from Accelerated Segment Test (FAST), Binary Robust Independent Elementary Features (BRIEF) descriptors as used, e.g., in Orientated FAST and Rotated BRIEF (ORB), Binary Robust Invariant Scalable Keypoints (BRISK), etc. In some embodiments, rather than use these conventional features, features may be generated using a neural network (e.g., from values in a layer of a UNet network, using the approach specified in the 2021 paper “LoFTR: Detector-Free Local Feature Matching with Transformers” available as arXiv™ preprint arXiv™:2104.00680v1 and by Jiaming Sun, Zehong Shen, Yuang Wang, Hujun Bao, and Xiaowei Zhou, using the approach specified in “SuperGlue: Learning Feature Matching with Graph Neural Networks”, available as arXiv™ preprint arXiv™:1911.11763v2 and by Paul-Edouard Sarlin, Daniel DeTone, Tomasz Malisiewicz, and Andrew Rabinovich, etc.). Such customized features may be useful when applied to a specific internal body context, specific camera type, etc.

The same type of features may be generated (or retrieved if previously generated) for previously considered images at block 540. For example, if M is 1, then only the previous image will be considered. In some embodiments, every previous image may be considered (e.g., M is N−1) similar to the “BundleFusion” approach of Dai, et al. The features generated at block 540 may then be matched with those features generated at block 535. These matching correspondences determined at block 545 may themselves then be used to determine a pose estimate at block 550 for the Nth image, e.g., by finding an optimal set of rigid camera transforms best aligning the features of the N through N-M images.

In contrast to feature-matching based pose estimation subprocess 530a, the depth-determination based pose estimation process 530b employs one or more machine learning architectures to determine a pose and a depth estimation. For example, in some embodiments, estimation process 530b considers the image N and the image N−1, submitting the combination to a machine learning architecture trained to determine both a pose and depth frame for the image, as indicated at block 555 (though not shown here for clarity, one will appreciate that where there are not yet any preceding images, or when N=1, the system may simply wait until a new image arrives for consideration; thus block 505 may instead initialize N to M so that an adequate number of preceding images exist for the analysis). One will appreciate that a number of machine learning architectures which may be trained to generate both a pose and depth frame estimate for a given visual image in this manner. For example, some machine learning architectures, similar to subprocess 530a, may determine the depth and pose by considering as input not only the Nth image frame, but by considering a number of preceding image frames (e.g., the Nth and N−1th images, the Nth through N-M images, etc.). However, one will appreciate that machine learning architectures which consider only the Nth image to produce depth and pose estimations also exist and may also be used. For example, block 555 may apply a single image machine learning architecture produced in accordance with various of the methods described in the paper “Digging Into Self-Supervised Monocular Depth Estimation” referenced above. The Monodepth2 self-supervised model described in that paper may be trained upon images depicting the endoscopic environment. Where sufficient real-world endoscopic data is unavailable for this purpose, synthetic data may be used. Indeed, while Godard et al.'s self-supervised approach with real world data does not contemplate using exact pose and depth data to train the machine learning architecture, synthetic data generation may readily facilitate generation of such parameters (e.g., as one can advance the virtual camera through a computer generated model of an organ in known distance increments) and may thus facilitate a fully supervised training approach rather than the self-supervised approach of their paper (though synthetic images may still be used in the self-supervised approach, as when the training data includes both synthetic and real-world data). Such supervised training may be useful, e.g., to account for unique variations between certain endoscopes, operating environments, etc., which may not be adequately represented in the self-supervised approach. Whether trained via self-supervised, fully supervised, or prepared via other training methods, the model of block 555 here predicts both a depth frame and pose for a visual image. One will appreciate a variety of methods for supplementing unbalanced synthetic and real-world datasets, including, e.g., the approach described in the 2018 paper “T2Net: Synthetic-to-Realistic Translation for Solving Single-Image Depth Estimation Tasks” available as arXiv™ preprint arXiv™:1808.01454v1 and by Chuanxia Zheng, Tat-Jen Cham, and Jianfei Cai, the approach described in the 2019 paper “Geometry-Aware Symmetric Domain Adaptation for Monocular Depth Estimation” available as arXiv™ preprint arXiv™:1904.01870v1 and by Shanshan Zhao, Huan Fu, Mingming Gong, and Dacheng Tao, the approach described in the paper “Unpaired Image-to-Image Translation using Cycle-Consistent Adversarial Networks” available as arXiv™ preprint arXiv™:1703.10593v7 and by Jun-Yan Zhu, Taesung Park, Phillip Isola, and Alexei A. Efros, and any suitable neural style transfer approach, such as that described in the paper “Deep Photo Style Transfer” available as arXiv™ preprint arXiv™:1703.07511v3 and by Fujun Luan, Sylvain Paris, Eli Shechtman, and Kavita Bala (e.g., suitable for results suggestive of photorealistic images).

Thus, as processing continues to block 560, the system may have available the pose determined at block 550, a second pose determined at block 555, as well as the depth frame determined at block 555. The pose determined at block 555 may not be the same as the pose determined at block 550, given their different approaches. If block 550 succeeded in finding a pose (e.g., a sufficiently large number of feature matches), then the process may proceed with the pose of block 550 and the depth frame generated at block 555 in the subsequent processing (e.g., transitioning to block 580).

However, in some situations, the pose determination at block 550 may fail. For example, where features failed to match at block 545, the system may be unable to determine a pose at block 550. While such failures may happen in the normal course of image acquisition, given the great diversity of body interiors and conditions, such failures may also result, e.g., when the operator moved the camera too quickly, resulting in a blurring of the Nth frame, making it difficult or impossible for features to be generated at block 535. Instrument occlusions, biomass occlusions, smoke (e.g., from a cauterizing device), or other irregularities may likewise result in either poor feature generation or poor feature matching. Naturally, if such an image is subsequently considered at block 545 it may again result in a failed pose recognition. In such situations, at block 560 the system may transition to block 565, preparing the pose determined at block 555 to serve in the place of the pose determined at block 550 (e.g., adjusting for differences in scale, format, etc., though substitution at block 575 without preparation may suffice in some embodiments) and making the substitution at block 575. In some embodiments, during the first iteration from block 515, as no previous frames exist with which to perform a match in the process 530a at block 540, the system may likewise rely on the pose of block 555 for the first iteration.

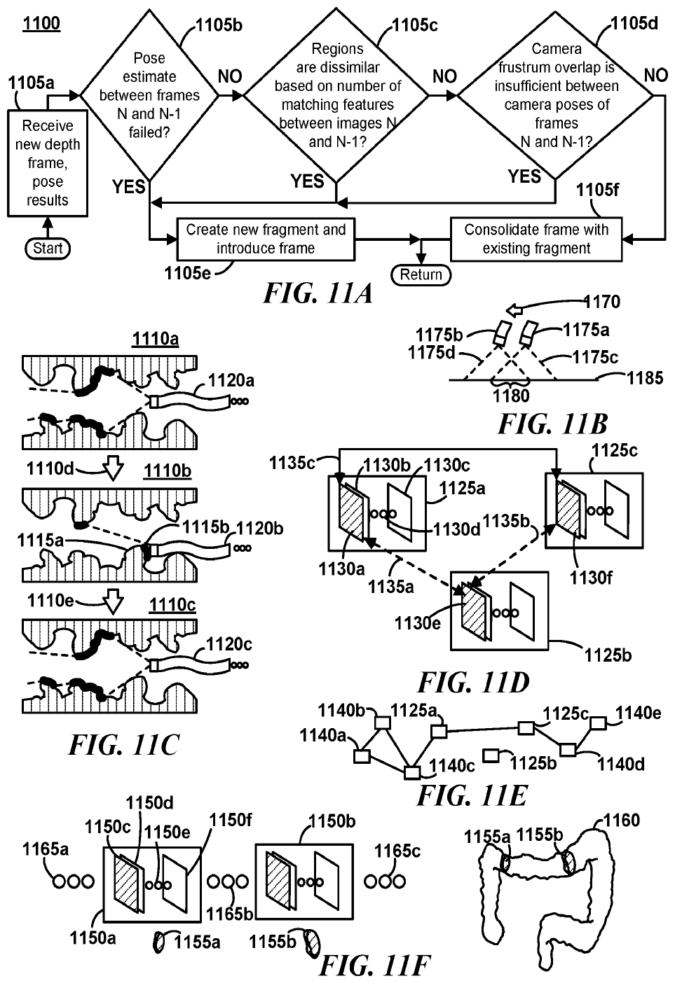

At block 580, the system may determine if the pose (whether from block 550 or from block 555) and depth frame correspond to the existing fragment being generated, or if they should be associated with a new fragment. A variety of methods may be used for determining when a new fragment is to be generated. In some embodiments, new fragments may simply be generated after a fixed number (e.g., 20) of frames have been considered. In other embodiments, the number of matching features at block 545 may be used as a proxy for region similarity. Where a frame matches many of the features in its immediately prior frame, it may be reasonable to assign the corresponding depth frames to the same fragment (e.g., transition to block 590). In contrast, where the matches are sufficiently few, one may infer that the endoscope has moved to a substantially different region and so the system should begin a new fragment at block 585a. In addition, the system may also perform global pose network optimization and integration of the previously considered fragment, as described herein, at block 585b (for clarity, one will recognize that the “local” poses, also referred to as “coarse” poses, of blocks 550 and 555 are relative to successive frames, whereas the “global” pose is relative to the coordinates of the model as a whole). One example method for performing block 580 is provided herein with respect to the process 1100 of FIG. 11A.

With the depth frame and pose available, as well as their corresponding fragment determined, at block 590 the system may integrate the depth frame with the current fragment using the pose estimate. For example, simultaneous localization and mapping (SLAM) may be used to determine the depth frame's pose relative to other frames in the fragment. As organs are often non-rigid, non-rigid methods such as that described in the paper “As-rigid-as-possible surface modeling” by Olga Sorkine and Marc Alexa, appearing in Symposium on Geometry processing. Vol. 4. 2007, may be used. Again, one will appreciate that the exact methodology specified therein need not be used in every embodiment. Similarly, some embodiments may employ methods from the DynamicFusion approach specified in the paper “DynamicFusion: Reconstruction and tracking of non-rigid scenes in real-time” by Richard A. Newcombe, Dieter Fox, and Steven M. Seitz, appearing in Proceedings of the IEEE conference on computer vision and pattern recognition, 2015. DynamicFusion may be appropriate as many of the papers referenced herein do not anticipate the non-rigidity of body tissue, nor the artifacts resulting from respiration, patient motion, surgical instrument motion, etc. The canonical model referenced in that paper would thus correspond to the keyframe depth frame described herein. In addition to integrating the depth frame with its peer frames in the fragment, at block 595, the system may append the pose estimate to a collection of poses associated with the frames of the fragment for future consideration (e.g., the collective poses may be used to improve global alignment with other fragments, as discussed with respect to block 570).

Once all the desired images from the video have been processed at block 515, the system may transition to block 570 and begin generating the complete, or intermediate, model of the organ by merging the one or more newly generated fragments with the aid of optimized pose trajectories determined at block 595. In some embodiments, block 570 may be foregone, as global pose alignment at block 585b may have already included model generation operations. However, as described in greater detail herein, in some embodiments not all fragments may be integrated into the final mesh as they are acquired, and so block 570 may include a selection of fragments from a network (e.g., a network like that described herein with respect to FIG. 11E).

### Example Data Processing Operations Pre-Processing Variation

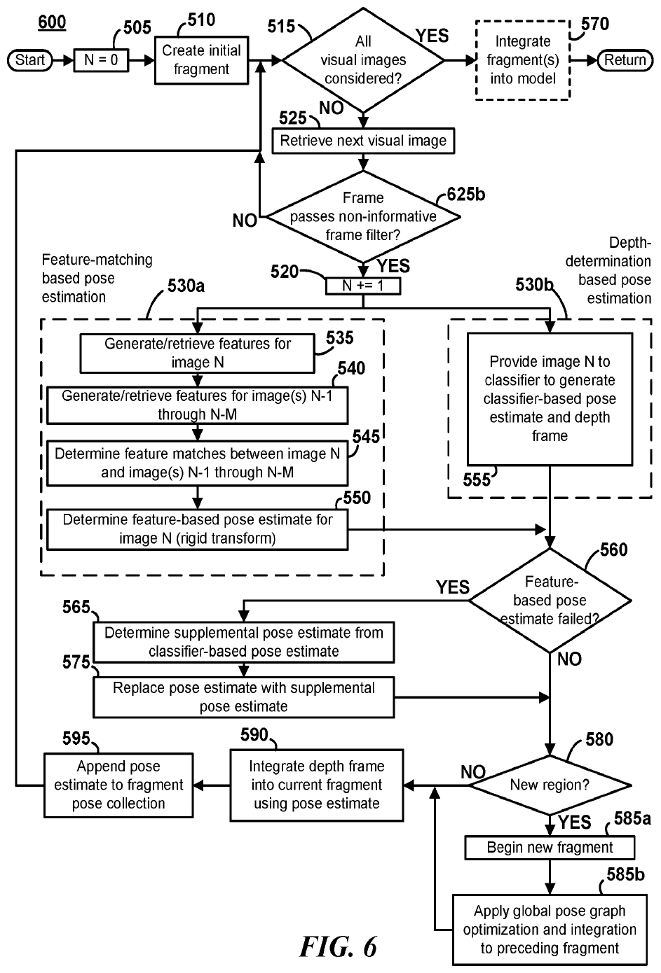

FIG. 6 is a flow diagram illustrating a pre-processing variation of the process of FIG. 5, as may be implemented in some embodiments. Particularly, while most of processing operations may remain generally as described above, this example process 600 may also seek to exclude visual images unsuitable for downstream processing, thereby greatly improving the system's efficiency and effectiveness. Not only does the consideration of unsuitable visual images consume valuable resources to no purpose, but it may also result in the system trying to reconcile the downstream processing's results for the unsuitable image with the results of other, possibly suitable, images. Thus, consideration of a single unsuitable image may impede the proper consideration of subsequent suitable images. Accordingly, at block 625b, the system may provide the image to a non-informative frame filter, such as a neural network as described herein, to assess whether the image is suitable for downstream processing. If the filter finds that the image is “non-informative”, the downstream processing may be foregone for the image and the next visual image considered instead, as indicated.

In contrast, if the filter finds the visual image to be suitable, the count N of informative visual images may be incremented at block 620, and the visual image frame then processed by two distinct subprocesses, a feature-matching based pose estimation subprocess 530a and a depth-determination based pose estimation subprocess 530b, in parallel, as previously discussed. For clarity, here, the counter value N refers to the total count of the image frames found to be informative, rather than all the image frames simply (thus, N−1 refers to the previous image which was found to be informative and not necessarily to the previous image simply chronologically).

### Example End-to-End Data Processing Pipeline

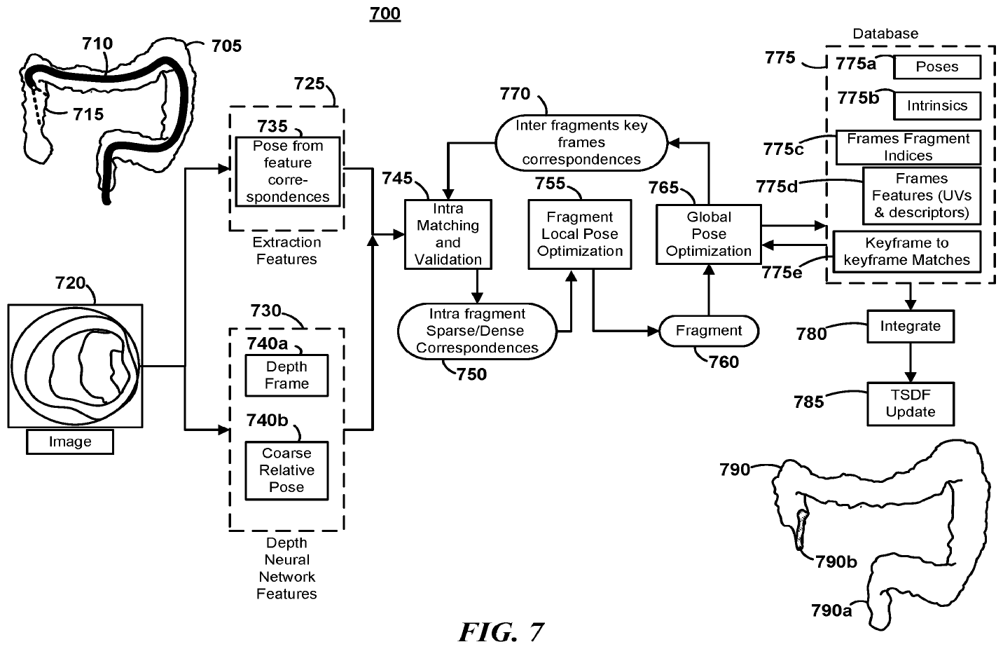

For additional clarity, FIG. 7 is a processing pipeline 700 for generating at least a portion of a three-dimensional model of a large intestine from a colonoscope data capture, as may be implemented in some embodiments. Again, while a large intestine is shown here to facilitate understanding, one will appreciate that the embodiments contemplate other organs and interior structures of patient 120.

Here, as a colonoscope 710 progresses through an actual large intestine 705, the camera or depth sensor may bring new regions of intestine 705 into view. At the moment depicted in FIG. 7, the region 715 of the intestine 705 is within view of the endoscope camera resulting in a two-dimensional visual image 720 of the region 715. The computer system may use the image 720 to generate both extraction features 725 (corresponding to process 530a) and depth neural network features 730 (corresponding to process 530b). In this example, the extraction features 725 produce the pose 735. Conversely, the depth neural network features 730 may include a depth frame 740a and pose 740b (though a neural network generating pose 740b may be unnecessary in embodiments where the pose 735 is always used).

As discussed, the computer system may use pose 735 and depth frame 740a in matching and validation operations 745, wherein the suitability of the depth frame and pose are considered. At blocks 750 and 755, the new frame may be integrated with the other frames of the fragment by determining correspondences therebetween and performing a local pose optimization. When the fragment 760 is completed, the system may align the fragment with previously collected fragments via global pose optimization 765 (corresponding, e.g., to block 585b). The computer system may then perform global pose optimization 765 upon the fragment 760 to orient the fragment 760 relative to the existing model. After creation of the first fragment, the computer system may also use this global pose to determine keyframe correspondences between fragments 770 (e.g., to generate a network like that described herein with respect to FIG. 11E).

Performance of the global pose optimization 765 may involve referencing and updating a database 775. The database may contain a record of prior poses 775a, camera calibration intrinsics 775b, a record of frame fragment indices 775c, frame features including corresponding UV texture map data (such as the camera images acquired of the organ) 775d, and a record of keyframe to keyframe matches 775e (e.g., like the network of FIG. 11E). The computer system may integrate 780 the database data (e.g., corresponding to block 570) at the conclusion of the examination, or in real-time during the examination, to update 785 or produce a computer generated model of the organ, such as a TSDF representation 790. In this example, the system is operating in real-time and is updating the preexisting portion of the TSDF model 790a with a new collection of voxels (or, e.g., corresponding vertices and textures where the model is a polygonal mesh) 790b corresponding to the new fragment 760 generated for the region 715.

### Example End-to-End Data Processing Pipeline Preprocessing Variation

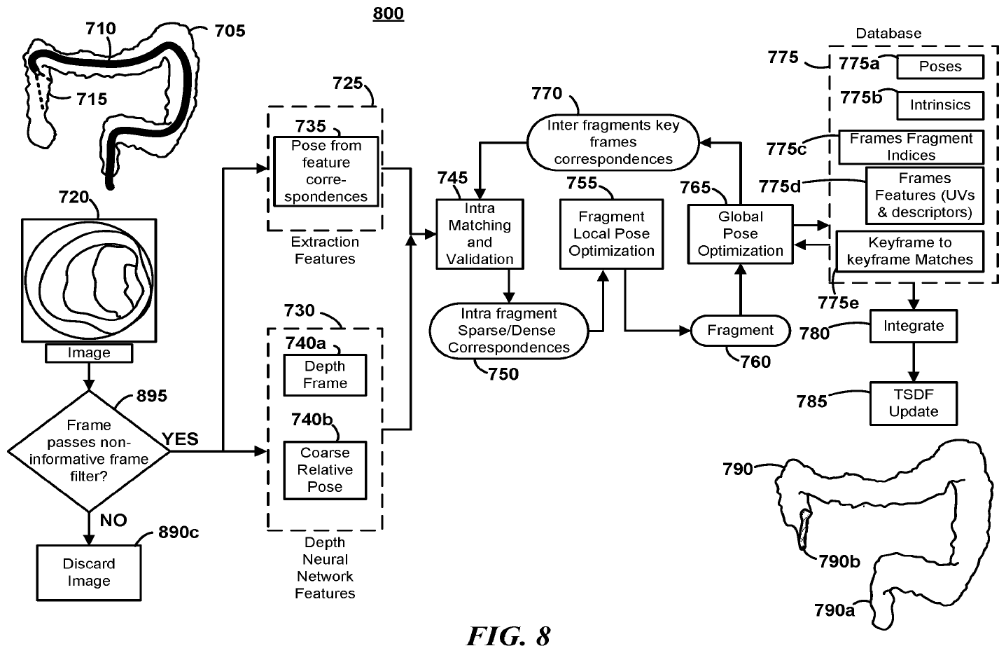

For additional clarity, FIG. 8 is an example processing pipeline 800 depicting a pre-processing variation of the pipeline of FIG. 7, as may be implemented in some embodiments. Similar to the additional pre-processing of FIG. 6. as the colonoscope 710 progresses through an actual large intestine 705, the camera or depth sensor may bring new regions of intestine 705 into view. As previously discussed, at the moment depicted in FIG. 7, the region 715 of the intestine 705 is within view of the endoscope camera resulting in a two-dimensional visual image 720 of the region 715. Following confirmation 895 that the image is suitable for localization and mapping (e.g., as was discussed with respect to block 625b), rather than discard the frame 890c, the computer system may use the image 720 to generate both extraction features 725 (corresponding to process 530a) and depth neural network features 730 (corresponding to process 530b), as discussed previously.

### Example End-to-End Data Processing Pipeline—Example Pose and Depth Pipeline

One will appreciate a number of methods for determining the coarse relative pose 740b and depth map 740a (e.g., at block 555). Naturally, where the examination device includes a depth sensor, the depth map 740a may be generated directly from the sensor (naturally, this may not produce a pose 740b). However, many depth sensors impose limitations, such as time of flight limitations, which may mitigate the sensor's suitability for in-organ data capture. Thus, it may be desirable to infer pose and depth data from visual images, as most examination tools will already be generating this visual data for the surgeon's review in any event.

Inferring pose and depth from an visual image can be difficult, particularly where only monocular, rather than stereoscopic, image data is available. Similarly, it can be difficult to acquire enough of such data, with corresponding depth values (if needed for training), to suitably train a machine learning architecture, such as a neural network. Some techniques do exist for acquiring pose and depth data from monocular images, such as the approach described in the “Digging Into Self-Supervised Monocular Depth Estimation” paper referenced herein, but these approaches are not directly adapted to the context of the body interior (Godard et al.'s work was directed to the field of autonomous driving) and so do not address various of this data's unique challenges.

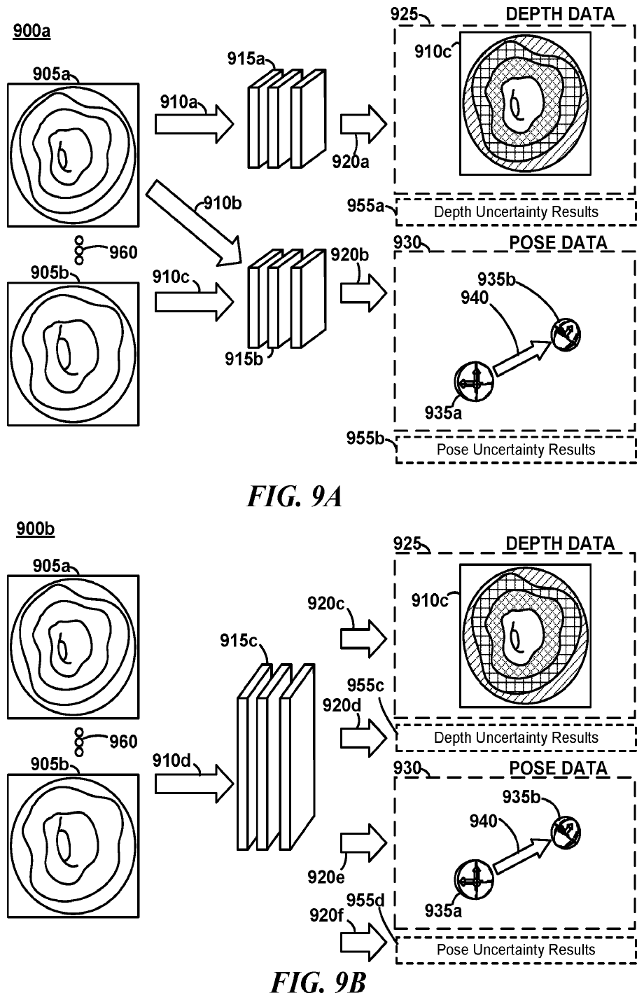

FIG. 9A depicts an example processing pipeline 900a for acquiring depth and pose data from monocular images in the body interior context. Here, the computer system considers two temporally successive image frames from an endoscope camera, initial image capture 905a and subsequent capture 905b after the endoscope has advanced forward through the intestine (though, as indicated by ellipsis 960, one will readily appreciate variations where more than two successive images are employed and the inputs to the neural networks may be adjusted accordingly; similarly one will appreciate corresponding operations for withdrawal and other camera motion). In the pipeline 900a, a computer system supplies 910a initial image capture 905a to a first depth neural network 915a configured to produce 920a a depth frame representation 925 (corresponding to depth data 740a). One will appreciate that where more than two images are considered, image capture 905a may be, e.g., the first of the images in temporal sequence. Similarly, the computer system supplies 910b, 910c both image 905a and image 905b to a second pose neural network 915b to produce 920b a coarse pose estimate 930 (corresponding to coarse relative pose 740b). Specifically, network 915b may predict a transform 940 explaining the difference in view between both image 905a (taken from orientation 935a) and image 905b (taken from orientation 935b). One will appreciate that in embodiments where more than two successive images are considered, the transform 940 may be between the first and last of the images, temporally. Where more than two input images are considered, all of the input images may be provided to network 915b.

Thus, in some embodiments, depth network 915a may be a UNet-like network (e.g., a network with substantially the same layers as UNet) configured to receive a single image input. For example, one may use the DispNet network described in the paper “Unsupervised Monocular Depth Estimation with Left-Right Consistency” available as an arXiv™ preprint arXiv™:1609.03677v3 and by Clement Godard, Oisin Mac Aodha, and Gabriel J. Brostow for the depth determination network 915a. As mentioned, one may also use the approach from “Digging into self-supervised monocular depth estimation” described above for the depth determination network 915a. Thus, the depth determination network 915a may be, e.g., a UNet with a ResNet(50) or ResNet(101) backbone and a DispNet decoder. Some embodiments may also employ depth consistency loss and masks between two frames during training as in the paper “Unsupervised scale-consistent depth and ego-motion learning from monocular video” available as arXiv™ preprint arXiv™:1908.10553v2 and by Jia-Wang Bian, Zhichao Li, Naiyan Wang, Huangying Zhan, Chunhua Shen, Ming-Ming Cheng, and Ian Reid and methods described in the paper “Unsupervised Learning of Depth and Ego-Motion from Video” appearing as arXiv™ preprint arXiv™:1704.07813v2 and by Tinghui Zhou, Matthew Brown, Noah Snavely, and David G. Lowe.

Similarly, pose network 915b (when, e.g., the pose is not determined in parallel with one of the above approaches for network 915a) may be a ResNet “encoder” type network (e.g., a ResNet(18) encoder), with its input layer modified to accept two images (e.g., a 6-channel input to receive image 905a and image 905b as a concatenated RGB input). The bottleneck features of this pose network 915b may then be averaged spatially and passed through a 1×1 convolutional layer to output 6 parameters for the relative camera pose (e.g., three for translation and three for rotation, given the three-dimensional space). In some embodiments, another 1×1 head may be used to extract two brightness correction parameters, e.g., as was described in the paper “D3VO: Deep Depth, Deep Pose and Deep Uncertainty for Monocular Visual Odometry” appearing as an arXiv™ preprint arXiv™:2003.01060v2 by Nan Yang, Lukas von Stumberg, Rui Wang, and Daniel Cremers. In some embodiments, each output may be accompanied by uncertainty values 955a or 955b (e.g., using methods as described in in the D3VO paper). One will recognize, however, that many embodiments generate only pose and depth data without accompanying uncertainty estimations. In some embodiments, pose network 915b may alternatively be a PWC-Net as described in the paper “PWC-Net: CNNs for Optical Flow Using Pyramid, Warping, and Cost Volume” available as an arXiv™ preprint arXiv™:1709.02371v3 by Deqing Sun, Xiaodong Yang, Ming-Yu Liu, and Jan Kautz or as described in the paper “Towards Better Generalization: Joint Depth-Pose Learning without PoseNet” available as an arXiv™ preprint arXiv™:2004.01314v2 by Wang Zhao, Shaohui Liu, Yezhi Shu, and Yong-Jin Liu.

One will appreciate that the pose network may be trained with supervised or self-supervised approaches, but with different losses. In supervised training, direct supervision on the pose values (rotation, translation) from the synthetic data or relative camera poses, e.g., from a Structure-from-Motion (SfM) model such as COLMAP (described in the paper “Structure-from-motion revisited” appearing in Proceedings of the IEEE conference on computer vision and pattern recognition. 2016 by Johannes L. Schonberger, and Jan-Michael Frahm) may be used. In self-supervised training, photometric loss may instead provide the self-supervision.

Some embodiments may employ the auto-encoder and feature loss as described in the paper “Feature-metric Loss for Self-supervised Learning of Depth and Egomotion” available as arXiv™ preprint arXiv™:2007.10603v1 and by Chang Shu, Kun Yu, Zhixiang Duan, and Kuiyuan Yang. Embodiments may supplement this approach with differentiable fisheye back-projection and projection, e.g., as described in the 2019 paper “FisheyeDistanceNet: Self-Supervised Scale-Aware Distance Estimation using Monocular Fisheye Camera for Autonomous Driving” available as arXiv™ preprint arXiv™:1910.04076v4 and by Varun Ravi Kumar, Sandesh Athni Hiremath, Markus Bach, Stefan Milz, Christian Witt, Clement Pinard, Senthil Yogamani, and Patrick Mader or as implemented in the OpenCV™ Fisheye camera model, which may be used to calculate back-projections for fisheye distortions. Some embodiments also add reflection masks during training (and inference) by thresholding the Y channel of YUV images. During training, the loss values in these masked regions may be ignored and in-painted using OpenCV™ as discussed in the paper “RNNSLAM: Reconstructing the 3D colon to visualize missing regions during a colonoscopy” appearing in Medical image analysis 72 (2021): 102100 by Ruibin Ma, Rui Wang, Yubo Zhang, Stephen Pizer, Sarah K. McGill, Julian Rosenman, and Jan-Michael Frahm.

Given the difficulty in acquiring real-world training data, synthetic data may be used in generating instances of some embodiments. In these example implementations, the loss for depth when using synthetic data may be the “scale invariant loss” as introduced in the 2014 paper “Depth Map Prediction from a Single Image using a Multi-Scale Deep Network” appearing as arXiv™ preprint arXiv™:1406.2283v1 and by David Eigen, Christian Puhrsch, and Rob Fergus. As discussed above, some embodiments may employ a general-purpose Structure-from-Motion (SfM) and Multi-View Stereo (MVS) pipeline COLMAP implementation, additionally learning camera intrinsics (e.g., focal length and offsets) in a self-supervised manner, as described in the 2019 paper “Depth from Videos in the Wild: Unsupervised Monocular Depth Learning from Unknown Cameras” appearing as arXiv™ preprint arXiv™:1904.04998v1 by Ariel Gordon, Hanhan Li, Rico Jonschkowski, and Anelia Angelova. These embodiments may also learn distortion coefficients for fisheye cameras.

Thus, though networks 915a and 915b are shown separately in the pipeline 900a, one will appreciate variations wherein a single network architecture may be used to perform both of their functions. Accordingly, for clarity, FIG. 9B depicts a variation wherein a single network 915c receives all the input images 910d (again, ellipsis 960 here indicates that some embodiments may receive more than two images, though one will appreciate that many embodiments will receive only two successive images). As before, such a network 915c may be configured to output 920c, 920d, 920e, 920f the depth prediction 925, pose prediction 930, and in some embodiments, one or more uncertainty predictions 955c, 955d (e.g., determining uncertainty as in D3VO, though one will readily appreciate variations). Separate networks as in pipeline 900a may simplify training, though some deployments may benefit from the simplicity of a single architecture as in pipeline 900b.

### Example End-to-End Data Processing Pipeline—Example Pose and Depth Pipeline—Example Training

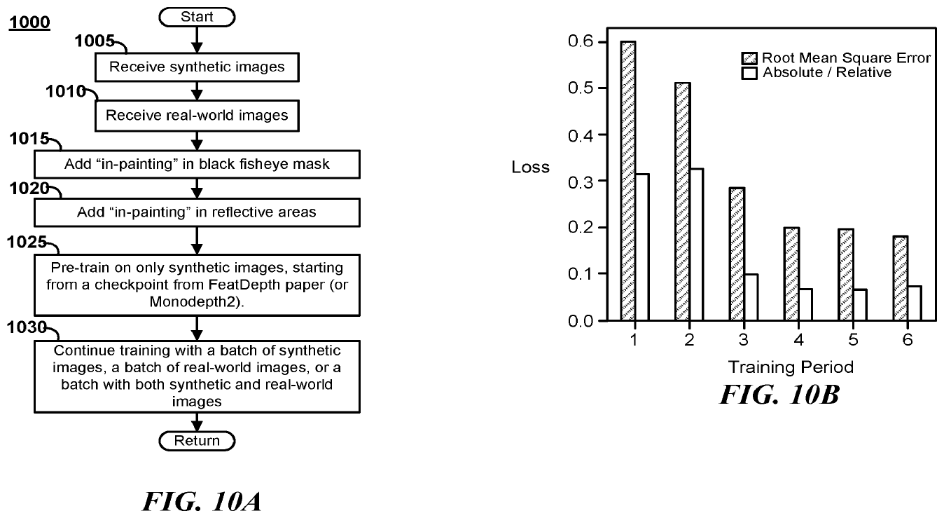

FIG. 10A is a flow diagram illustrating various operations in an example neural network training process 1000, e.g., for training each of networks 915a and 915b. At block 1005 the system may receive any synthetic images to be used in training and validation. Similarly at block 1010, the system may receive the real world images to be used in training and validation. These datasets may be processed at blocks 1015 and 1020, in-painting reflective areas and fisheye borders. One will appreciate that, once deployed, similar preprocessing may occur upon images not already adjusted in this manner.

At block 1025 the networks may be pre-trained upon synthetic images only, e.g., starting from a checkpoint in the FeatDepth network of the “Feature-metric Loss for Self-supervised Learning of Depth and Egomotion” paper or the Monodepth2 network of the “Digging Into Self-Supervised Monocular Depth Estimation” paper referenced above. Where FeatDepth is used, one will appreciate that an auto-encoder and feature loss as described in that paper may be used. Following this pre-training, the networks may continue training with data comprising both synthetic and real data at block 1030. In some embodiments, COLMAP sparse depth and relative camera pose supervision may be here introduced into the training.

FIG. 10B is a bar plot depicting an exemplary set of training results for the process of FIG. 10A.

### Example Fragment Management

As discussed with respect to process 500, the depth frame consolidation process may be facilitated by organizing frames into fragments (e.g., at block 585a) as the camera encounters sufficiently distinct regions, e.g., as determined at block 580. An example process for making such a determination at block 580 is depicted in FIG. 11A. Specifically, after receiving a new depth frame at block 1105a (e.g., as generated at block 555) the computer system may apply a collection of rules or conditions for determining if the depth frame or pose data is indicative of a new region (precipitating a transition to block 1105e, corresponding to a “YES” transition from block 580) or if the frame is instead indicative of a continuation of an existing region (precipitating a transition to block 1105f, corresponding to a “NO” transition from block 580).

In the depicted example, the determination is made by a sequence of conditions, the fulfillment of any one of which results in the creation of a new fragment. For example, with respect to the condition of block 1105b, if the computer system fails to estimate a pose (e.g., where no adequate value can be determined, or no value with an acceptable level of uncertainty) at either block 550 or at block 555, then the system may begin creation of a new fragment. Similarly, the condition of block 1105c may be fulfilled when too few of the features (e.g., the SIFT or ORB features) match between successive frames (e.g., at block 545), e.g., less than an empirically determined threshold. In some embodiments, not just the number of matches, but their distribution may be assessed at block 1105c, as by, e.g., performing a Singular Value Decomposition (SVD) of the depth values organized into a matrix and then checking the two largest resulting eigenvalues. If one eigenvalue is not significantly larger than the other, the points may be collinear, suggesting a poor data capture. Finally, even if a pose is determined (either via the pose from block 550 or from block 555), the condition of block 1105d may also serve to “sanity” check that the pose is appropriate by moving the depth values determined for that pose (e.g., at block 555) to an orientation where they can be compared with depth values from another frame. Specifically, FIG. 11B illustrates an endoscope moving 1170 over a surface 1185 from a first position 1175a to a second position 1175b with corresponding fields of view 1175c and 1175d respectively. One would expect depth values between the region 1180 to overlap, as shown by the portion 1180 of the surface 1185. The overlap in depth values may be verified by moving the values in one capture to their corresponding position in the other capture (as considered at block 1105d). A lack of similar depth values within a threshold may be indicative of a failure to acquire a proper pose or depth determination.

One will appreciate that while the conditions of blocks 1105a, 1105b, and 1105c may serve to recognize when the endoscope travels into a field of view sufficiently different from that in which it was previously situated, the conditions may also indicate when smoke, biomass, body structures, etc. obscure the camera's field of view. To facilitate the reader's comprehension of these latter situations, an example circumstance precipitating such a result is shown in the temporal series of cross-sectional views in FIG. 11C. Endoscopes may regularly collide with portions of the body interior during an examination. For example, initially at time 1110a the colonoscope may be in a position 1120a (analogous to the previous discussion with respect to FIGS. 4A-C) with a field of view suitable for pose determination. Unfortunately, patient movement, inadvertent operator movement, etc., may transition 1110d the configuration to the new state of time 1110b, where the camera collides with a ridge wall 1115a resulting in a substantially occluded view, mostly capturing a surface region 1115b of the ridge. Naturally, in this orientation 1120b, the endoscope camera captures few, if any, pixels useful for any proper pose determination. When the automated examination system or operator recovers 1110e at time 1110c the endoscope may again be in a position 1120c with a field of view suitable for making a pose and depth determination.

One will appreciate that, even if such a collision only occurs over the course of a few seconds or less, the high frequency with which the camera captures visual images may precipitate many new visual images. Consequently, the system may attempt to produce many corresponding depth frames and poses, which may themselves be assembled into fragments in accordance with the process 500. Undesirable fragments, such as these, may be excluded by the process of global pose graph optimization at block 585b and integration at block 570. Fortuitously, this exclusion process may itself also facilitate the detection and recognition of various adverse events during procedures.

Specifically, FIG. 11D is a schematic collection of fragments 1125a, 1125b, and 1125c. Fragment 1125a may have been generated while the colonoscope was in the position of time 1110a, fragment 1125b may have been generated while the colonoscope was in the position of time 1110b, and fragment 1125c may have been generated while the colonoscope was in the position of time 1110c. As discussed, each of fragments 1125a, 1125b, and 1125c may include an initial keyframe 1130a, 1130e, and 1130f respectively (here, the keyframe is the first frame inserted into the fragment). Thus, for clarity, the first frame of fragment 1125a is keyframe 1130a, frame 1130b was the next acquired frame, and so on (intermediate frames being represented by ellipsis 1130d) until the final frame 1130c is reached. During global pose estimation at block 585b, the computer system may have recognized sufficient feature (e.g., SIFT or ORB) or depth frame similarity between keyframes 1130a and 1130f that they could be identified as depicting connected regions of depth values (represented by link 1135c). This is not surprising given the similarity of the field of view at times 1110a and 1110c. However, the radical character of the field of view at time 1110b, makes keyframe 1130e too disparate from either keyframe 1130a or 1130f to form a connection (represented by the nonexistent links 1135a and 1135b).

Consequently, as shown in the hypothetical graph pose network of FIG. 11E, viable fragments 1140a, 1140b, 1140c, 1140d, 1140e and 1125a, and 1125c may form a network with reachable nodes based upon their related keyframes, but fragment 1125b may remain isolated. One will appreciate that frame 1125b may coincidentally match other frames on occasion (e.g., where there are multiple defective frames resulting from the camera pressed against a flat surface, they may all resemble one another), but these defective frames will typically form a much smaller, isolated (or more isolated) network from the primary network corresponding to capture of the internal body structure. Consequently, such frames may be readily identified and removed from the model generation process at block 570.

Though not shown in FIG. 11D, one will appreciate that, in addition to depth values, each frame in a fragment may have a variety of metadata, including, e.g., the corresponding visual image(s), estimated pose(s) associated therewith, timestamp(s) at which the acquisition occurred, etc. For example, as shown in FIG. 11F, fragments 1150a and 1150b are two of many fragments appearing in a network (the presence of preceding, succeeding, and intervening fragments represented by ellipses 1165a, 1165c, and 1165b, respectively). Fragment 1150a includes the frames 1150c, 1150d, and 1150f (ellipsis 1150e reflecting intervening frames) and the first temporally acquired frame 1150c is designated as the keyframe. From the frames in fragment 1150a one may generate an intermediate model such as a TSDF representation 1155a (similarly, one may generate an intermediate model, such as TSDF 1155b, for the frames of fragment 950b). With such intermediate TSDFs available, integration of fragments into a partial or complete model mesh (or remain in TSDF form) 1160 may proceed very quickly (e.g., at block 570 or integration 780), which may be useful for facilitating real-time operation during the surgery.

### Overview—Reference Geometries for Surgical Instrument Kinematics

Various of the disclosed embodiments provide precise metrics for monitoring surgical instrument kinematics relative to a reference geometry, such as a geometric structure, or manifold, embedded in the Euclidean space in which a three-dimensional model of the patient's interior resides. Though specific reference to colonoscopy will often be made herein to facilitate a consistent presentation for the reader's understanding, one will appreciate the application of many of the disclosed systems and methods, mutatis mutandis, to other surgical environments, such as prostatectomy, bronchial pulmonary analysis, general laparoscopic procedures, etc. Thus, various embodiments may be applied to a surgical instrument navigating, e.g., along the lungs over a pre-procedure computed tomography (CT) scan to detect polyps. As in the colonoscopy context, the system may estimate the centerline geometric structure of the route to navigate during such a pulmonary procedure.

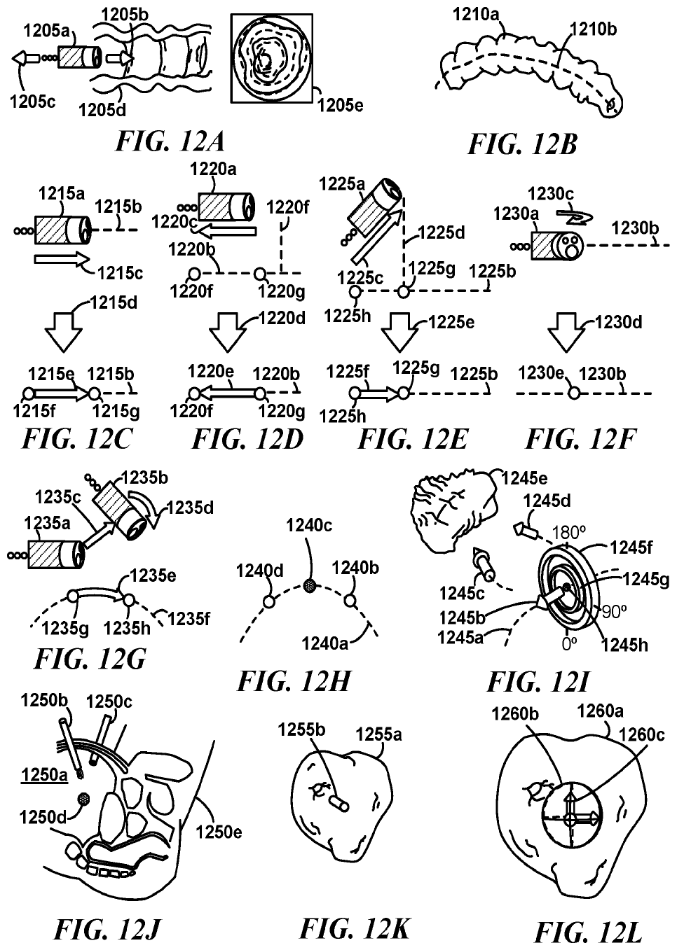

FIG. 12A is a schematic cross-sectional view of a colonoscope 1205a within a portion of a colon 1205d, and the resulting colonoscope camera field of view 1205e, during either an advancing 1205b (proceeding further into the colon away from the point of insertion, e.g., the anus) or a withdrawing 1205c (moving the colonoscope back towards the point of insertion) motion, as may occur in some embodiments. The nature of the colonoscope's 1205a movement within the colon may have implications for the quality and character of the surgical procedure. For example, movement of the colonoscope 1205a within the colon too quickly may precipitate motion blur, as shown in the field of view 1205e.

The significance of such movement may also depend upon spatial or temporal factors. For example, spatially, movement of the colonoscope too near a sidewall of the colon may be undesirable near an injured region of the colon more so than en route to that portion through healthy regions. Regarding temporal context, a higher movement speed during insertion may be appropriate where the priority is to reach and examine a region of interest, but the same speed may be inappropriate during withdrawal past regions which were incompletely inspected during the advance. Such motion profile thresholds may be determined, e.g., by Key Opinion Leaders (KOLs). Consequently, a rigorous and precise system for monitoring these and other situations would be desirable for a number of downstream operations, including machine learning operations as well as simply informing the surgical operator of the instrument's present kinematic behavior.

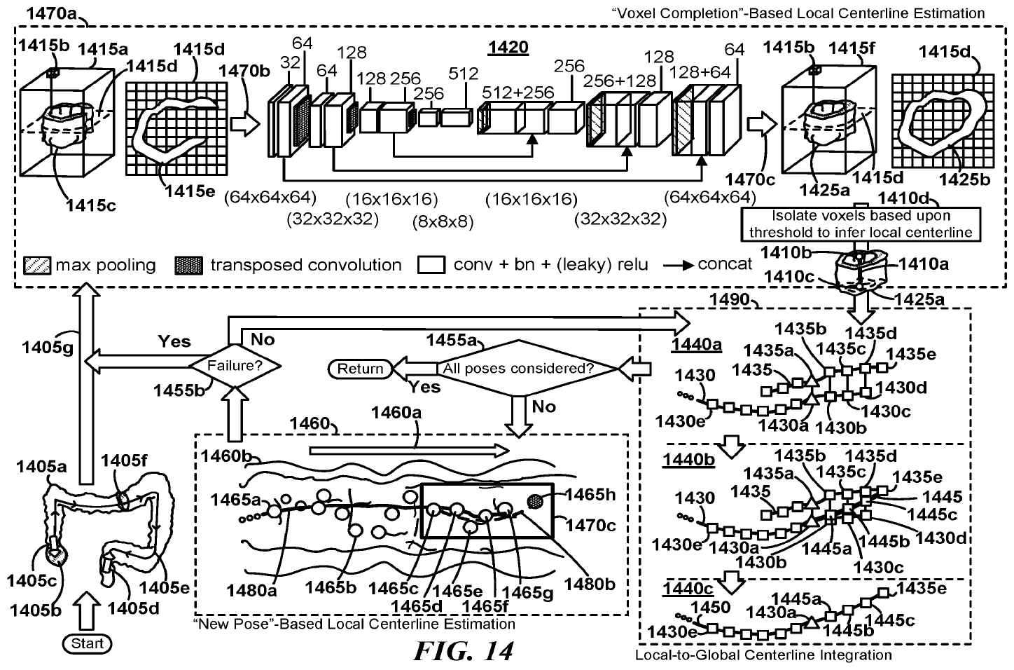

To provide robust kinematics data so as to facilitate such considerations across surgical procedures, various embodiments contemplate the creation of reference geometries, such as lines, circles, hemispheres, spheres, etc. within the same Euclidean space in which the patient interior may be represented. For example, FIG. 12B is a schematic three-dimensional model 1210a of a portion of an organ (created, e.g., using the localization and mapping systems and methods described herein) with a medial centerline axis reference geometry 1210b. Here, the centerline 1210b of the three-dimensional model of the organ may be used as a consistent reference for interpreting surgical instrument kinematics. The centerline may be the medial axis for all or a portion of the model along the model's length. Movement both upon, or relative, to the centerline 1210b and orthogonal, or residual, to the centerline 1210b may be considered. One will appreciate a number of methods for creating the centerline 1210b. For example, the colon model 1210a may be averaged or collapsed. As discussed in greater detail herein, however, (e.g., with reference to FIG. 14) some embodiments determine the centerline 1210b using an iterative, section-based approach, which may produce a centerline 1210b generally invariant to the complexity of the colon sidewall surface. Such invariance may be especially useful given the wide physical differences between the lungs, colons, esophagi, etc. of different patients.

One will appreciate that such comparisons of an instrument's motion relative to the centerline 1210b may occur both during or after the surgical procedure, e.g., as when the centerline is created at the end of the surgical procedure and then the previously recorded positions of the surgical instruments are used to determine the relative kinematics. However, real-time creation of the centerline during the surgery may often be desirable, as this may facilitate direct kinematics feedback to the surgical team. One will also appreciate, as described in greater detail herein, that while the same reference geometry may be used for assessing the entirety of the surgical procedure in some embodiments, in other embodiments the reference geometry may change over time, e.g., as the organ is deformed, as context and requirements change, etc. and more than one geometry may be used at the same time.

To facilitate the reader's comprehension, FIG. 12C is a schematic side perspective view of a colonoscope camera 1215a in an advancing orientation relative to a centerline reference geometry 1215b. Specifically, as the colonoscope moves in the direction of vector 1215c, directly along the centerline 1215b, then the projection 1215d of this vector 1215c upon the centerline will be the same vector 1215e. Thus, if the colonoscope moves forward exactly upon, or parallel to, the centerline, its velocity vector in Euclidean space may be the same vector, in direction and magnitude, upon the centerline. The closest point upon the centerline for the camera's previous position in this example was the point 1215f and the closest point for its new position is the point 1215g.

Similarly, as shown in FIG. 12D, a withdrawal motion vector 1220c of the colonoscope camera 1220a above the centerline 1220b (at a distance indicated by the reference line 1220f), may result in a projected 1220d vector 1220e upon the centerline 1220b, which is the same as the withdrawal motion vector 1220c. Thus, where the motion of the colonoscope camera is parallel with the centerline, then the motion vector of the camera, whether advancing on (as in FIG. 12C) or off the centerline axis, or withdrawing on or off (as in FIG. 12D) the centerline axis, the speed of the camera's motion will be the same as the speed upon the centerline. The closest point upon the centerline for the camera's previous position in this example was the point 1220g, and the closest point for its new position is the point 1220f.

In contrast, for further clarity, one will appreciate that movement of the colonoscope camera, which is not parallel with the centerline, as shown in FIG. 12E, may result in relative motion projected upon the centerline which differs from that of the actual camera in three-dimensional space. Specifically, in this example, the camera 1225a is in a non-parallel orientation above the centerline 1225b, and thus its motion forward in this orientation produces a motion vector 1225c. As indicated by reference line 1225d, projection 1225e of the vector 1225c upon the centerline 1225b results in the smaller movement vector 1225f. Here, the point 1225g is the closest point upon the centerline for the camera's new position, whereas the point 1225h was the closest point at the camera's previous orientation. As will be discussed in greater detail herein, portions of the vector 1225c which do not appear in the projection upon the centerline (e.g., the motion along the reference line 1225d), referred to as residual kinematics, may also be determined in some embodiments, as such motion may have great significance in various contexts (e.g., leaving the centerline and approaching a sidewall at an inopportune time or location).

Again, for further clarity, one will appreciate that, as shown in FIG. 12F, there may be forms of residual motion, which are not orthogonal to the centerline. In this example, the colonoscope 1230a is neither advancing, withdrawing, nor moving lateral to the centerline 1230b, but only rotating 1230c. Thus, as there is no translational component, the projection 1230d produces no vector upon the centerline 1230b, and no relative kinematics data. However, some embodiments may monitor the orientation of the camera's center of field of view relative to the centerline 1230b, as the relation between these two vectors may be informative of the examined regions of the colon sidewall. Thus, even though the translational position of the camera has not changed between two successive image captures in FIG. 12F, and consequently, the closest point upon the centerline remains the point 1230e for both frames, the system may note the change in relative angles between the centerline and the center of the field of view in the two orientations (e.g., with cross or dot product of the two vectors) as part of the residual kinematics.

For further clarity, some embodiments determine movement of the surgical instrument, such as the camera, based upon its translation or rotation (e.g., as described in FIG. 12F) above a certain threshold relative to previous valid frame. For example, EON. 1 indicates how translational movement of the camera may be assessed:

$$translation motion = {‖ {T}_{t} - {T}_{last valid frame} ‖}^{2} \qquad ( 1 )$$

where Tₜ is the translation vector relative to a global origin of the camera at the current time t and T_{last valid frame} is the translation vector of the camera relative to a global origin at the time of a previous valid frame capture.

Rotation of the camera may then be determined in accordance with EON. 2

$$rotation motion = arccos ( \frac{Tr ( \hat{R} ) - 1}{2} ) \qquad ( 2 )$$

$$where$$

$$\hat{R} = {‖ {R}_{t} * {R}_{last valid frame}^{- 1} ‖}^{2} \qquad ( 3 )$$

Motion may then be found to exist if either the translation exceeds a threshold value or the rotation exceeds a threshold value. Once rotational motion is found to have exceeded the threshold, the system may determine the relationship between the rotated field of view to the axis vector of the centerline.

One will appreciate that the centerline may not always take the form of a “straight line,” e.g., where the colon assumes a curved structure. Thus, FIG. 12G depicts a curved centerline 1235f. One will appreciate that, just as in the previously discussed figures, combinations of translations and rotations of the colonoscope relative to the centerline may still precipitate a cumulative relative projective result upon the centerline. Here, for example, translational 1235c and rotational 1235d movement from a first orientation 1235a to a second 1235b may result in a cumulative projection upon the centerline 1235f of the vector 1235e. Accordingly, the point 1235g on the centerline was closest to the camera in the orientation 1235a and the point 1235h on the centerline is closest to the camera in the orientation 1235b. Here, the system may record both the projected relative translation along the centerline manifold, as well as the residual change in rotation of the camera. One will appreciate that a manifold herein refers to a three-dimensional object embedded within Euclidean space with a line or surface upon which projections of surgical instrument motion may be made.

Naturally, the rate at which orientations of the camera are compared may affect the granularity of the projected movement upon the centerline. In some embodiments, the comparison rate may be the same as the framerate at which the images are acquired by the camera. Often, the capture rate may be fast enough that the relative and residual kinematics data is of adequate quality. However, as shown in FIG. 12H, in some embodiments and situations it may be desirable to interpolate projected positions upon the centerline so as to infer kinematics at higher resolutions. For example, if a projected position of the camera upon the centerline 1240a at a first time corresponds to the point 1240b (corresponding, e.g., to point 1235g or to point 1220g, determined at a first time), and at the next moment of capture, projection upon the centerline 1240a is at the point 1240d (corresponding, e.g., to point 1235h or to point 1220f, determined a second time successive to the first), rather than infer a direct line motion from the point 1240b to the point 1240d in Euclidean space, the system may interpolate the movement along the centerline manifold. Thus, the projected motion between the points 1240b and 1240d may pass through the point 1240c. Where encoders and other mechanical sensor configurations are available, the system may compare the projected, interpolated centerline motion with that derived from the encoders. However, often it will be beneficial to infer motion from the camera images only and so a framerate may be selected to be commensurate with the maximum velocities expected of the surgical instrument so as to ensure capture of all the desired motions. Often, motion too quick for accurate determination of a reference projection may also be too quick for proper depth frame determinations.

One will appreciate that such interpolations may likewise occur for rotations. That is, where the rotation of the camera relative to the centerline at the first time of capture associated with the point 1240b is different from the relative rotation at the later time of capture associated with the point 1240d, the system may record any intermediate values as a linear interpolation of the two (e.g., taking dot products from corresponding portions of the interpolated centerline).

In some embodiments, appropriate determination of a reference geometry, such as a centerline, and successive orientations of a surgical instrument relative thereto may enable a number of useful downstream actions and assessments. For example, FIG. 12I is a schematic perspective view of an orientation upon a centerline reference geometry 1245a with radial spatial contexts as may occur in some embodiments. Here, the centerline 1245a passes a region of interest, specifically a disease artifact 1245e, such as a polyp, tumor, etc. Regions around the centerline 1245a may be associated with different contextual functions. For example, a first region 1245g around the centerline 1245a may indicate an upper bound for movement when advancing or withdrawing the colonoscope. Moving the colonoscope outside this region may trigger a warning or alarm during these stages of the surgical procedure. Similarly, a region 1245f may be used for the same purpose in wider regions of the colon. Thus, during such “travel” phases of the procedure, a colonoscope at the position 1245h may be encouraged to advance along an appropriate vector 1245b.

Such contextual spatial and locational monitoring need not be limited to regions radially extending from the centerline. Motions orthogonal or away from the centerline may likewise be taken into consideration. The system may consider not only change in orientation relative to the closest portion of the centerline, but relative to portions of the centerline previously encountered in the surgical procedure or which will be encountered in the future of the operation. For example, as depicted in FIG. 12I, upon encountering the artifact 1245e, each of the off-centerline paths 1245c and 1245d may be better than maintaining an orientation 1245b upon the path, as they will provide more direct and closer fields of view of the artifact 1245e. However, turning backward to perceive the artifact as in path 1245c may be less ideal than approaching the artifact with smaller deviation from the centerline as in the path 1245d. Thus, a high fidelity reference geometry not only facilitates precise kinematic metrics upon the geometry itself, but contextual metrics outside the geometry, such as the orthogonally radial and context-aware metrics described here.

Again, while many of the embodiments disclosed herein are consistently described with reference to the colonoscopy context for clarity of comprehension, one will appreciate that other embodiments may be applied in other contexts and with other surgical instruments. For example, FIG. 12J is a schematic cross-sectional view of a patient's pelvic region 1250e during a robotic surgical procedure as may occur in some embodiments. Here, a first 1250b and second 1250c surgical instrument may be inserted via respective portals into a laparoscopically inflated cavity 1250a of a patient interior. Reference geometry embedded manifolds may be determined within the Euclidean space of the cavity 1250a, including centerlines, curved surfaces around regions of interest, etc. Here, a central sphere 1250d at the center of the cavity 1250a provides a manifold upon which to project motions of one or both of the instruments 1250b and 1250c.

The reference geometry embedded manifolds may be selected based upon the structure of the modeled interior region of the patient, the nature of the surgical procedure, or both. For example, in FIG. 12K, a cylindrical reference geometry 1255b is located at the center of a three-dimensional model of a cavity 1255a, the elongated axis of the cylinder oriented relative to a region of interest, such that projections of surgical instrument movement may provide information relevant to the surgical procedure under consideration. Similarly, as shown in FIG. 12K, reference geometries may be oriented with an awareness of the structure of the patient interior region. For example, here, while the reference geometry is a sphere 1260b, the reference geometry may maintain a consistent orientation across surgeries relative to landmarks within the three-dimensional model of the cavity 1260a. That is, the axis 1260c shown here pointing upward, will likewise point upward in other models. In this manner, projected surgical instrument motions upon the surface of the sphere 1260b may readily be compared across surgeries.

### Example Medial Axis Centerline Estimation—System Processes

Naturally, more precise and consistently generated reference geometries, such as centerlines, may better enable more precise operations, including, e.g., circumference selection and assessments of surgical instrument kinematics. Such consistency may be useful when analyzing and comparing surgical procedure performances. Accordingly, with specific reference to the example of creating centerline reference geometries in the colonoscope context, various embodiments contemplate improved methods for determining the centerline based upon the localization and mapping process, e.g., as described previously herein.

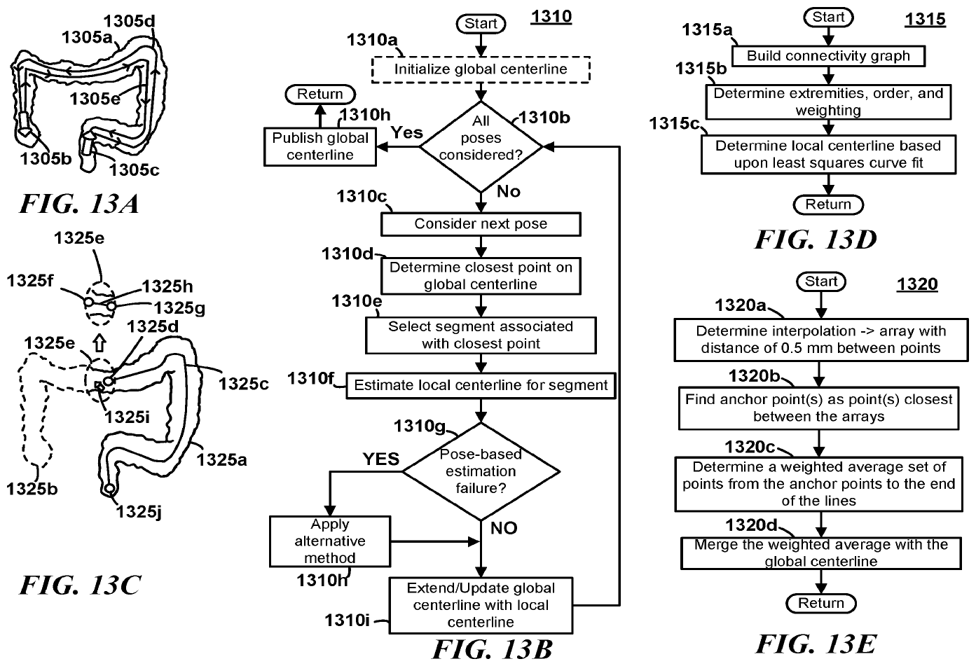

To facilitate the reader's understanding, FIG. 13A is a schematic three-dimensional model of a colon 1305a. As described above, during the surgical procedure the colonoscope may begin in a position and orientation 1305c within the colon 1305a, and advance 1305d forward, collecting depth frames, and iteratively generating a model (e.g., as discussed with respect to FIG. 7) until reaching a terminal position 1305b (though, in some embodiments, localization and mapping may occur only during withdrawal). During withdrawal 1305e, the trajectory may, for the most part, be reversed from that of the advance 1305d, with the colonoscope beginning in the position and orientation 1305b, at or near the cecum, and then concluding in the position and orientation 1305c. During the withdrawal 1305e, additional depth frame data captures may facilitate improvements to the fidelity of the three-dimensional model of the colon (and consequently any reference geometries derived from the model, as when the centerline is estimated as a center moment of model circumferences).

While some embodiments seek to determine a centerline and corresponding kinematics throughout both advance 1305d and withdrawal 1305e, in some embodiments, the reference geometry may only be determined during withdrawal 1305e, when at least a preliminary model is available to aid in the geometry's creation. In other embodiments, the system may wait until after the surgery, when the model is complete, before determining the centerline and corresponding kinematics data from a record of the surgical instrument's motion.

By approaching centerline creation via an iterative approach, wherein centerlines for locally considered depth fames are first created and then conjoined with an existing global centerline estimation for the model, reference geometries suitable for determining kinematics feedback during the advance 1305d, during the withdrawal 1305e, or during post-surgical review, may be possible. For example, during advance 1305d, or withdrawal 1305e, the projections upon the reference geometry may be used to inform the user that their motions are too quick. Such warnings may be provided and be sufficient even though the available reference geometry and model are presently less accurate than they will be once mapping is entirely complete. Conversely, higher fidelity operations, such as comparison of the surgeon's performance with other practitioners, may only be performed once higher fidelity representations of the reference geometry and model are available. Access to a lower fidelity representation, may still suffice for real-time feedback.

Specifically, FIG. 13B is a flow diagram illustrating various operations in an example medial centerline estimation process 1310, as may be implemented in some embodiments, facilitating the iterative merging of local centerline determinations with a global centerline determination. Specifically, at block 1310a, the system may initialize a global centerline data structure. For example, at position and orientation 1305b prior to withdrawal 1305e, if no centerline has yet been created, then the system may prepare a first endpoint of the centerline as the current position of the colonoscope, or as the position at 1305c, with an extension to an averaged value of the model sidewalls. Conversely, if a centerline was already created during the advance 1305d, then that previous centerline may be taken as the current, initialized global centerline. Finally, if the data capture is just beginning (e.g., prior to advance 1305d) and the colonoscope is in the position and rotation 1305c, then global centerline endpoint may be the current position of the colonoscope, with a small extension along the axis of the current field of view. As will be discussed in greater detail with respect to FIG. 14, machine learning systems for determining local centerlines from the model TSDF may be employed during initialization at block 1305a.

At block 1310b, the system may iterate over acquired localization poses for the surgical camera (e.g., as they are received during advance 1305d or withdrawal 1305e), until all the poses have been considered, before publishing the “final” global centerline at block 1310h (though, naturally, kinematics may be determined using the intermediate versions of the global centerline, e.g., as determined at block 1310i). Each camera pose considered at block 1310c may be, e.g., the most current pose captured during advance 1305d, or the next pose to be considered in a queue of poses ordered chronologically by their time of acquisition.

At block 1310d, the system may determine the closest point upon the current global centerline relative to the position of the pose considered at block 1310c. At block 1310e, the system may consider the model values (e.g., voxels in a TSDF format) within a threshold distance of the closest point determined at block 1310d, referred to herein as a “segment,” associated with the closest point upon the centerline determined at block 1310d. In some embodiments, dividing the expected colon length by the depth resolution and multiplying by an expected review interval, e.g., 6 minutes, may indicate the appropriate distance around a point for determining a segment boundary, as this distance corresponds to the appropriate “effort” of review by an operator to inspect the region.

For clarity, with reference to FIG. 13C, a global centerline 1325c may have already been generated for a portion 1325a of the model of the colon. The model may itself still be in a TSDF format, and may be accordingly represented in a “heatmap” or other voxel format. The portion 1325b of the model may not yet have a centerline, e.g., because that portion of the model does not yet exist, as during an advance 1305d, or may exist, but may not yet be considered for centerline determination (e.g., during post-processing after the procedure).

Thus, the next pose 1325i (here, represented as an arrow in three-dimensional space corresponding to the position and orientation of the camera looking toward the upper colon wall) may be considered, e.g. as the pose was acquired chronologically and selected at block 1310c. The nearest point on the centerline 1325c to this pose 1325i as determined at block 1310d is the point 1325d. A segment is then the portion of the TSDF model within a threshold distance of the point 1325d, shown here as the TSDF values appearing the region 1325e (shown separately as well to facilitate the reader's comprehension). Accordingly, the segment may include all, a portion, or none of the depth data acquired via the pose 1325i. At block 1310f, the system may determine the “local” centerline 1325h for the segment in this region 1325e, including its endpoints 1325f and 1325g. The global centerline (centerline 1325c) may be extended at block 1310i with this local centerline 1325h (which may result in the point 1325f now becoming the furthest endpoint of the global centerline opposite the global centerline's start point 1325j). As will be discussed in greater detail with respect to FIG. 14, in some embodiments, at block 1310g, the system may consider whether pose-based local centerline estimation failed at block 1310f, and if so apply an alternative method for local centerline determination at block 1310h (e.g., application of a neural network and centerline determination logic). Such alternative methods, while more robust and more accurate than the pose-based estimation, may be too computationally intensive for continuous use during real-time applications, such as during the surgical procedure.

One will appreciate a variety of methods for performing the operations of block 1310f. For example, FIG. 13D is a flow diagram illustrating various operations in an example process 1315 for estimating such a local centerline segment. As will be described in greater detail herein with reference to FIG. 14, pose-based local centerline estimation for a given segment may generally comprise three operations, summarized here in blocks 1315a, 1315b, and 1315c. At block 1315a, the system may build a connectivity graph for poses appearing in the segment (e.g., the most recent poses ahead of the field of view during withdrawal 1305e, or the most recent poses behind the field of view during the advance 1305d). The connectivity graph may be used to determine the spatial ordering of the poses before fitting the local centerline. For each pose, the shortest distance to the “oldest” (as by time of capture) pose along the graph may be computed using a “breadth-first search” and the order then determined based upon those distances. The closest pose in the graph may be selected as the first pose in the ordering, the second closest pose in the graph as the second pose in the ordering, etc.

Using this graph between the poses, at block 1315b, the system may then determine extremal poses (e.g., those extremal voxels most likely to correspond to the points 1325f and 1325g), the ordering of poses along a path between these extremal points, and the corresponding weighting associated with the path (weighting based, e.g., upon the TSDF density for each of the voxels). Order and other factors, such as pose proximity, may also be used to determine weights for interpolation (e.g., as constraints for fitting a spline). The local centerline may also be estimated using a least squares fit, using B-splines, etc.

Finally, at block 1315c, the system may determine the local centerline 1325h based upon, e.g., a least-square fit (or other suitable interpolation, such as a spline) between the extremal endpoint poses determined at block 1315b. Determining the local centerline based upon such a fit may facilitate a better centerline estimation than if the process continued to be bound to the discretized locations of the poses. The resulting local centerline may later to be merged with the global center line as described herein (e.g., at block 1310i and process 1320).

Similarly, a number of approaches are available to implement the operations of block 1310i. For example, FIG. 13E is a flow diagram illustrating various operations in an example process 1320 for extending (or, mutatis mutandis, updating a preexisting portion) a global centerline (e.g., global centerline 1325c) with a segment's local centerline (e.g., local centerline 1325h), as may be implemented in some embodiments. Here, at block 1320a the system may determine a first “array” of points (a sequence of successive points along the longitudinal axis) upon the local centerline and a second array of points on the global centerline, e.g., points within 0.5 mm (or other suitable threshold, e.g., as adjusted in accordance with the colonoscope's speed based upon empirical observation) of one another. While such an array may be determined for the full length of the local and global centerlines, some embodiments determine arrays only for the portions appearing in or near the region under consideration (e.g., 1325e). As will be described in FIG. 14, the local centerline's array may be deliberately extended with an additional 1 cm worth of points, relative to the global centerline as a buffer.

At block 1320b, the system may then identify which pair of points, one from each of the two arrays, has a spatially closest pair of points relative to the other pairs, each of the pair of so-identified points referred to herein as an “anchor.” The anchors may thus be selected as those points where the local and global arrays most closely correspond. At block 1320c, the system may then determine a weighted average between the pairs of points in the arrays from the anchor point to the terminal end of the local centerline array (e.g., including the 1 cm buffer). The weighted average between these pairs of points may include the anchors themselves in some embodiments, though the anchors may only indicate the terminal point of the weighted average determination. Finally at block 1320d, the system may then determine the weighted average of the local and global centerlines around this anchor point.

### Example Medial Axis Centerline Estimation Process—Schematic Pipeline

To better facilitate the reader's comprehension of the example situations and processes of FIGS. 13A-E, FIG. 14 presents many of the same operations in a schematic operational pipeline, this time in the context of an embodiment wherein localization, mapping, and reference geometry estimation are applied only during withdrawal. Specifically, in this example, the operator has advanced the colonoscope to a start position without initiating centerline estimation (e.g., inspection of the colon may only occur during withdrawal, where the kinematics are most relevant, and so the operator is simply concerned, at least initially, with maneuvering the colonoscope to the proper start position), then performs centerline estimation throughout withdrawal. Again, in some embodiments, model creation may have occurred during the advance and the centerline may be created from all or only a portion of the model. In the depicted example, though, the centerline is to be calculated only during the withdrawal and, when possible, with the use of the poses, rather than relying upon the model's fidelity.

As shown following the start of the pipeline, the operator has advanced the colonoscope from an initial start position 1405d within the colon 1405a to a final position 1405c at and facing the cecum. From this final position 1405c the operator may begin to withdraw the colonoscope along the path 1405e. Having arrived at the cecum, and prior to withdrawal, the operator, or other team member, may manually indicate to the system (e.g., via button press) that the current pose is in the terminal position 1405c facing the cecum. However, in some embodiments automated system recognition (e.g., using a neural network) may be used to automatically recognize the position and orientation of the colonoscope in the cecum, thus precipitating automated initialization of the reference geometry creation process.

In accordance with block 1310a, the system may here initialize the centerline by acquiring the depth values for the cecum 1405b. These depth values (e.g., in a TSDF format and suitably organized for input into a neural network) may be provided 1405g to a “voxel completion based local centerline estimation” component 1470a, here, encompassing a neural network 1420 for ensuring that the TSDF representation is in an appropriate form for centerline estimation and post-completion logic in the block 1410d. Specifically, while holes may be in-filled by direct interpolation, a planar surface, etc., in some embodiments, a flood-fill style neural network 1420 may be used (e.g., similar to the network described in Dai, A., Qi, C. R., NieBner, M.: Shape completion using 3d-encoder-predictor cnns and shape synthesis. In: Proc. Computer Vision and Pattern Recognition (CVPR), IEEE (2017); one will appreciate that “conv” here refers to a convolutional layer, “bn” to batch normalization, “relu” to a rectified linear unit, and the arrows indicate concatenation of the layer outputs with layer inputs).

For example, in the TSDF voxel space 1415a (e.g., a 64×64×64 voxel grid), a segment 1415c is shown with a hole in its side (e.g., a portion of the colon not yet properly observed in the field of view for mapping). One familiar with the voxel format will appreciate that the larger region 1415a may be subdivided into cubes 1415b, referred to herein as voxels. While voxel values may be binary in some embodiments (representing empty space or the presence of the model), in some embodiments, the voxels may take on a range of values, analogous to a heat map, e.g., where the values may correspond to the probability a portion of the colon appears in the given voxel (e.g., between 0 for free space and 1 for high confidence that the colon sidewall is present).

For example, voxels inputted 1470b into a voxel point cloud completion network may take on values in according the EQN. 4:

$${H}_{input} [ v ] = tanh ( 0.2 * d ( v , {S}_{0} ) ) \qquad ( 4 )$$

and the output 1470c may take on values in accordance with EQN. 5

$${H}_{target} [ v ] = \frac{tanh ( 0.2 * d ( v , {S}_{1} ) )}{tanh ( 0.2 * d ( v , {S}_{1} ) ) + tanh ( 0.2 * d ( v , C ) )} \qquad ( 5 )$$

in each cases, where H[v] refers to the heatmap value for the voxel v, d(v,S₀)) is the Euclidean distance between the voxel v and the voxelized partial segment So, d(v,S₁) is the Euclidean distance between the voxel v and the voxelized complete segment S₁, and d(v, C) is the Euclidean distance between v and the voxelized estimated global centerline C. In this example, the input heatmap is zero at the position of the (partial) segment surface and increase towards 1 away from it, whereas the output heatmap is zero at the position of the (complete) segment surface and increases towards 1 at the position of the global centerline (converging to 0.5 everywhere else).

For clarity, if one observed an isolated plane 1415d in the region 1415a, one would see that the model 1415e is associated with many of the voxel values, though the region with a hole contains voxel values similar to, or the same as, empty space. By inputting the region 1415a into a neural network 1420, the system may produce 1470c an output 1415f with an in-filled TSDF section 1425a, including an infilling of the missing regions. Consequently, the planar cross-section 1415d of the voxel region 1415f is here shown with in-filled voxels 1425b. Naturally, such a network may be trained from a dataset created by gathering true-positive model segments, excising portions in accordance with situations regularly encountered in practice, then providing the latter as input to the network, and the former for validating the output.

A portion of the in-filled voxel representation of the section 1415f, may then be selected at block 1410d approximately corresponding to the local centerline location within the segment. For example, one may filter the voxel representation to identify the centerline portion by identifying voxels with values above a threshold, e.g., as in EQN. 6:

$$voxel value > 1 - δ . \qquad ( 6 )$$

where δ is an empirically determined threshold (e.g., in some embodiments taking on a value of approximately 0.15 centimeters).

For clarity, the result of the operations of the “voxel completion based local centerline estimation” component 1470a (including post-processing block 1410d) will be a local centerline 1410a (with terminal endpoints 1410b and 1410c shown here explicitly for clarity) for the in-filled segment 1425a. During the initialization of block 1310a, as there is no preexisting global centerline, there is no need to integrate the local centerline determined for the cecum TSDF 1405b with “voxel completion based local centerline estimation” component 1470a via local-to-global centerline integration operations 1490 (corresponding to block 1310i and the operations of the process 1320). Rather, the cecum TSDF's local centerline is the initial global centerline.

Now, as the colonoscope withdraws along the path 1405e, the localization and mapping operations disclosed herein may identify the colonoscope camera poses along the path 1405e. Local centerlines may be determined for these poses and then integrated with the global centerline via local centerline integration operations 1490. In theory, each of these local centerlines could be determined by applying the “voxel completion” based local centerline estimation component 1470a for each of their corresponding TSDF depth mesh (and, indeed, such an approach may be applied in some situations, such as post-surgical review, where computational resources are readily available). However, such an approach may be computationally expensive, complicating real-time applications. Similarly, certain unique mesh topologies may not always be suitable for application to such a component.

Accordingly, in some embodiments, pose-based local centerline estimation 1460 is generally performed. When complications arise, or metrics suggest that the pose-based approach is inadequate (e.g., the determined centerline is too closely approaching a sidewall), as determined at block 1455b, then the delinquent pose-based results may be replaced with results from the component 1470a. At block 1455b the system may, e.g., determine if the error between the interpolated centerline and the poses used to estimate the centerline exceeds a threshold. Alternatively, or additionally the system may periodically perform an alternative local centerline determination method (such as the component 1470a) and check for consensus with pose-based local centerline estimation 1460. Lack of consensus (e.g., a sum of differences between the centerline estimations above a threshold) may then precipitate a failure determination at block 1455b. While component 1470a may be more accurate than pose-based local centerline estimation 1460, component 1470a may be computationally expensive, and so its consensus validations may be run infrequently and in parallel with pose-based local centerline estimation 1460 (e.g., lacking consensus for a first of a sequence of estimations, component 1470a may be then applied for every other frame in the sequence, or some other suitable interval, and the results interpolated until the performance of pose-based local centerline estimation 1460 improves).

Thus, for clarity, after the initial application of the component 1470a to the cecum's TSDF 1405b, withdrawal may proceed along the path 1405e, applying the pose-based method 1460 until encountering the region 1405f. If pose-based local centerline estimation fails in this region 1405f, the TSDF for the region 1405f, and any successive delinquent regions, may be supplied to the component 1470a, until the global centerline is sufficiently improved or corrected that pose-based estimation local centerline estimation method 1460 may resume for the remainder of the withdrawal path 1405e.

At block 1455a in agreement with block 1310b the system may continue to receive poses as the operator withdraws along the path 1405e and extend the global centerline with each local centerline associated with each new pose. In greater detail, and was discussed with reference to block 1310f and the process 1315, the pose-based local centerline estimation 1460 may proceed as follows. As the colonoscope withdraws in the direction 1460a, through the colon 1460b, it will, as mentioned, produce a number of corresponding poses during localization, represented here as white spheres. For example, pose 1465a and pose 1465b correspond to previous positions of the colonoscope camera when withdrawing in the direction 1460a. Various of these previous poses may have been used in creation of the global centerline 1480a in its present form (an ellipsis at the leftmost portion of the centerline 1480a indicating that it may extend to the origination position in the cecum corresponding to the pose of position 1405c).

Having received a new pose, shown here as the black sphere 1465h, the system may seek to determine a local centerline, shown here in exaggerated form via the dashed line 1480b. Initially, the system may identify preceding poses within the threshold distance of the new pose 1465h, here represented as poses 1465c-g appearing within the bounding block 1470c. Though only six poses appear in the box in this schematic example, one will appreciate that many more poses would be considered in practice. Per the process 1315, the system may construct a connectivity graph between the poses 1465c-g and the new pose 1465h (block 1315a), determine the extremal poses in the graph (block 1315b, here the pose 1465c and new pose 1465h), and then determine the new local centerline 1480b, as the least squares fit, spline, or other suitable interpolation, between the extremal poses, as weighted by the intervening poses (block 1315c, that is, as shown, the new local centerline 1480b is the interpolated line, such as a spline with poses as constraints, between the extremal poses 1465c and 1465h, weighted based upon the intervening poses 1465d-g in accordance with the order identified at block 1315b).

Assuming the pose based centerline estimation of the method 1460 succeeded in producing a viable local centerline, and there is consequently no failure determination at block 1455b (corresponding to decision block 1310g), the system may transition to the local and global centerline integration method 1490 (e.g., corresponding to block 1310i and process 1320). Here, in an initial state 1440a, the system may seek to integrate a local centerline 1435 (e.g., corresponding to the local centerline 1480b as determined via the method 1460 or the centerline 1410a as determined by the component 1470a) with a global centerline 1430 (e.g., the global centerline 1480a). One will appreciate that the local centerline 1435 and the global centerline 1430 are shown here vertically offset to facilitate the reader's comprehension and may more readily overlap without so exaggerated a vertical offset in practice.

As was discussed with respect to block 1320a, the system may select points (shown here as squares and triangles) on each centerline and organize them into arrays. Here, the system has produced a first array of eight points for local centerline 1435, including the points 1435a-e. Similarly, the system has produced a second array of points for the global centerline 1430 (again, one will appreciate that an array may not be determined for the entire global centerline 1430, but only this terminal region near the local centerline, which is to be integrated). Comparing the arrays, the system has recognized pairs of points that correspond in their array positions, particularly, each of points 1435a-d correspond with each of points 1430a-d, respectively. In this example the correspondence is offset such that the point 1435e corresponding to the newest point of the local centerline (e.g., corresponding to the new pose 1465h) is not included in the corresponding pairs. One will appreciate that the correspondence may not be explicitly recognized, since the relationships may be inherent in the array ordering. As mentioned, the spacing of points in the array may be selected to ensure the desired correspondence, e.g., that the spacing is such that the point 1435d preceding the newest point of the local centerline 1435e, will appear in proximity to the endpoint 1430d of the global centerline. Accordingly, the spacing interval may not be the same on the local and global centerline following rapid, or disruptive, motion of the camera.

As mentioned at block 1320b, the system may then identify a closest pair of points between the two centerlines as anchor points. Here, the points 1435a and 1430a are recognized as being the closest pair of points (e.g., nearest neighbors), and so identified as anchor points, as reflected here in their being represented by triangles rather than squares.

Thus, as shown in state 1440b, and in accordance with block 1320c, the system may then determine the weighted average 1445 from the anchor points to the terminal points of the centerlines (the local centerline's 1435 endpoint 1435e dominating at the end of the interpolation), using the intervening points as weights (the new interpolated points 1445a-c falling upon the weighted average 1445, shown here for clarity). Finally, in accordance with block 1320d, and as shown in state 1440c, the weighted average 1445 may then be appended from the anchor point 1430a, so as to extend the old global centerline 1430 and create new global centerline 1450. For clarity, points preceding the anchor point 1430a, such as the point 1430e, will remain in the same position in the new global centerline 1450, as prior to the operations of the integration 1490.

Thus, the global centerline may be incrementally generated during withdrawal in this example via progressive local centerline estimation and integration with the gradually growing global centerline. Once all poses are considered at block 1455a, the final global centerline may be published for use in downstream operations (e.g., retrospective analysis of colonoscope kinematics). However, as described herein, because integration affects the portion of the global centerline following the anchor point 1430a, real-time kinematics analysis may be performed on the “stable” portion of the created global centerline preceding this region. As the stable portion of the global centerline may be only a small distance ahead or behind the colonoscope's present position, appropriate offsets may be used so that the kinematics generally correspond to the colonoscope's motion. Similarly, though this example has focused upon withdrawal exclusively to facilitate comprehension, application during advance (as well as to update a portion of, rather than extend, the global centerline) may likewise be applied mutatis mutandis.

By using the various operations described herein, one may create more consistent global centerlines (and associated kinematics data derived from the reference geometry), despite complex and irregular patient interior surfaces, and despite diverse variations between patient anatomies. As a consequence, the projected relative and residual kinematics data for the instrument motion may be more consistent between operations, facilitating better feedback and analysis.

### Context-Aware Kinematics Assessment

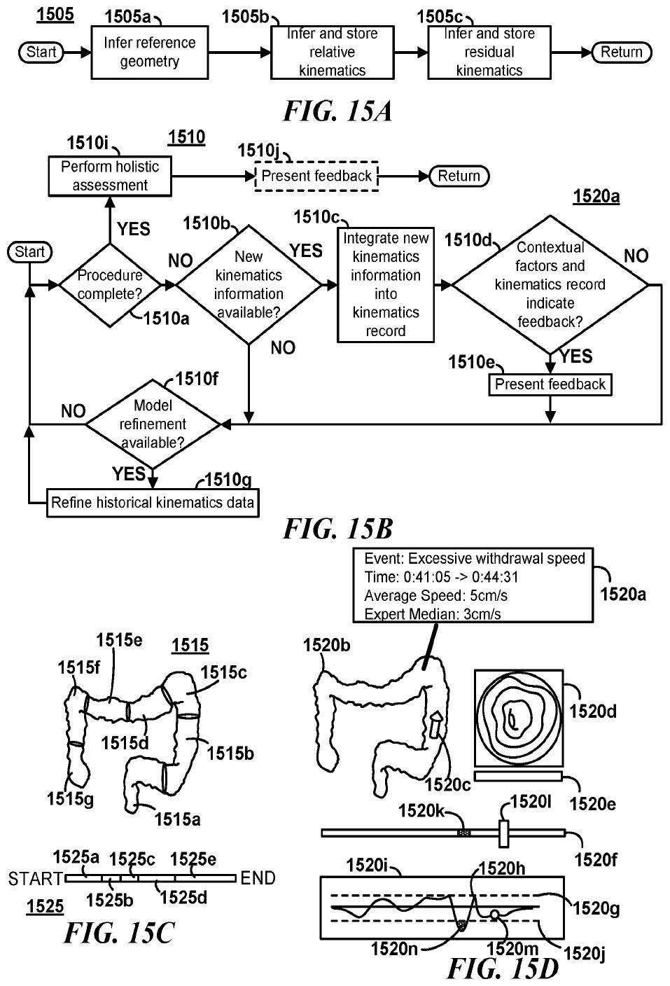

While specific examples have been provided above, once a reference geometry has been determined, in whatever suitable manner, the system may then assess the surgical instrument's kinematics relative to the geometry (e.g., both relative and residual kinematics). Specifically, at a high level, FIG. 15A is a flow diagram illustrating various operations in an example process 1505 for updating instrument kinematics relative to a reference geometry during a surgical procedure, as may be implemented in some embodiments. Generally, at block 1505a, the system may infer the reference geometry, e.g., using the centerline estimation methods disclosed herein. With the reference geometry available, the system may then consider the previously acquired pose information, encoder information, etc., to determine the relative kinematics of one or more surgical instruments as projected upon the reference geometry at block 1505b. As mentioned, the portion of the kinematics data not part of the relative kinematics, referred to as “residual kinematics” may likewise be inferred at block 1505c.

For further clarity, FIG. 15B is a flow diagram illustrating various operations in an example process 1510 for assessing kinematics information, as may be implemented in some embodiments. Over the course of the surgical procedure, at block 1510a, the system may consider if new kinematics information is available at block 1510b. For example, in colonoscopy, the system may wait for one of the rotation or translation thresholds of EQN. 1, EQN. 2, or EQN. 3 to be exceeded at block 1510b, or for a new depth frame to have been acquired at a new pose. Where new kinematics are determined to be available at block 1510b, then at block 1510c the new kinematics information may be integrated into the kinematic data record. In some embodiments, this may involve determination of the relative and residual kinematics as at blocks 1505b and 1505c, though such processing may be deferred in other embodiments.

At block 1510d, the system may consider whether contextual factors and the kinematics data record indicate a need for feedback to the surgical team. For example, motion too close to a colon sidewall, motion too quickly along the centerline near an anatomical artifact of interest, motion inappropriate for review of an anatomical artifact in a region, etc., may each trigger the presentation of feedback at block 1510e, such as an auditory warning or a graphical warning, e.g., in display 125, 150, 160a, etc.).

At block 1510f, the system may consider whether refinement of the model is possible. For example, during withdrawal 1305e, the camera's field of view may acquire better perspectives of previously encountered regions, facilitating the in-filling of holes in the model and possibly higher resolution models of the region. Improvements to these sections of the model may facilitate improved estimations of the centerline portion corresponding to those regions. The improved centerline may itself then facilitate improved relative and residual kinematics data calculations at block 1510g. As indicated, such refinement may be possible even if new kinematics data is not available. For example, model refinement may be possible, even without new kinematics data at block 1510b, when the system elects to iterate and consolidate previously acquired data frames, so as to improve the model of the patient interior.

Once all of the data for the surgical procedure has been acquired at block 1510a, at block 1510i the same or different computer system may initiate a holistic assessment of all the kinematics data and present feedback at block 1510j. One will appreciate that in addition to, or in lieu of, presenting feedback at block 1510j, the system may store the data, initiate a comparison with other instances of the surgical procedure by the same or different surgical operators, etc.

Again, combining knowledge of a surgical instruments' temporal and spatial location with the relative and residual kinematics data may facilitate a number of metrics and assessments, with applications both during and after the surgery. For example, FIG. 15C is a schematic representation of a colon model with spatial 1515 and temporal 1525 contextual regions as may be used in some embodiments. Specifically, a model may be divided into regions 1515a-g associated with different contextual factors, such as anatomical artifacts, surgical operations, procedure requirements, etc. Similarly, temporal regions 1525a-e, such as time limits, surgical tasks, etc. may be specified between the start and end of the surgical procedure. Surgical tasks may include discrete operations within a surgical procedure (e.g., region to be cauterized, excusing tumor, initiate withdrawal, etc.), recognizable by machine learning systems or by users.

Because localization may be performed throughout the surgical procedure, the system may consider the spatial 1515 and temporal 1525 contextual regions when considering whether to present feedback at blocks 1510d and 1510e. For example, preparatory insertion and withdrawal operations in the regions 1515a, whether early 1525a or late 1525e in the surgery, may commonly involve approaches to the colon sidewall, sudden changes in speed, etc. Consequently, the threshold for producing a warning may be smaller in these regions and times, then, e.g., in a region 1515e in the middle of the surgery, where sidewall encounters may cause greater damage or discomfort. Accordingly, the radial contexts 1245f, 1245g, etc. may take on varying significance with spatial and temporal context.

For further clarity, FIG. 15D provides a collection of GUI elements, as may be implemented in some embodiments. Such elements may be presented during or after the surgical procedure, as described in greater detail herein. A representation 1520b of the three-dimensional model of the colon may be presented, either in its partially created state during surgery or its final state after the surgery, as well as being presented either in the TSDF format, in a derived triangulated mesh representation, or other suitable representation. An indication 1520c of the surgical instrument's current position and orientation, here represented by an arrow, relative to the representation 1520b, may be used to indicate the instrument's orientation and location at the current time in the procedure, or in current time in the playback of the procedure. Popups, such as the excessive withdrawal speed popup 1520a, may indicate locations on the representation 1520b where undesirable kinematics behavior (whether relative or residual) was found to occur. Here, a timeline 1520f is likewise provided with an indication 15201 of the current time of playback. Portions of the timeline 1520f may be highlighted to provide information regarding the kinematics data, such as with changes in luminosity or hue (e.g., green for regions well within kinematic metric tolerances, orange and yellow for regions approaching a tolerance boundary, and red for regions where the tolerance boundary has been exceeded). Thus, the portion of the surgery precipitating the popup 1520a may also be identified by the highlighted region 1520k in the timeline (such as with a red hue indication).

In the example popup 1520a, information regarding the time during the surgery of the kinematics data event (at an interval slightly after 40 minutes into the surgery), the average speed of the operator during the event (“5 cm/s”) and reference data from similar practitioners (here, the median speed of “3 cm/s” for experts during corresponding portions of their procedures). While this example is for withdrawal speed, one will appreciate a number of events which may be triggered by assessments of the relative and residual kinematics data from the reference geometry. Thus, undesirable approaches toward a sidewall, undesirable approaches towards an artifact, undesirable motion of one instrument relative to another, constitute just some example events that may be recognized from the kinematics data and called to the attention of the surgical operator or reviewer.

The current image playback of the position and orientation corresponding to indication 1520c and time indicated by indicator 15201 may be shown in video playback region 1520d. A region 1520e may also provide information regarding the current kinematics assessment of the depicted frame (such as the present speed upon the centerline).

In some embodiments, the GUI may include a kinematics plot 1520i, depicting one of the metrics derived from the kinematic data (e.g., speed along the centerline, acceleration orthogonal to the centerline, etc.). Here, the GUI includes a plot of velocity 1520h along the centerline (positive values reflecting an advance and negative a withdrawal) throughout a portion of the procedure (though the x-axis here indicates temporal position, in some embodiments the velocity may be mapped to the length of the centerline itself, and the x-axis instead used to indicate points on the centerline), the current playback position shown by the indicator 1520m, corresponding to the indicator 15201, orientation 1520c, and current playback 1520d. Here, upper 1520g and lower 1520j kinematics metric boundaries may vary with the location and task being performed (though shown here as straight lines, one will appreciate that the thresholds may vary with time and spatial context). In this example, exceeding the lower bound 1520j in the region 1520n precipitated the excessive withdrawal speeds associated with the popup 1520a and region 1520k. The system may warn the operator that they are “going too fast” when the mapping produces as number of holes, the centerline motion is too fast for a reasonable assessment of the patient interior, camera blur prevents proper analysis or localization, etc. As speed orthogonal to the center line may indicate additional operations done during the procedure e.g., adjustments of the endoscope to inspect some regions behind folds, such residual kinematics above a threshold may be permitted (or associated with wider thresholds) only at times and in regions where they are to be expected.

With respect to colonoscopy in particular, as another example of a kinematics assessment, some embodiments may assume that a proper inspection of a portion of the colon may take approximately six minutes. Accordingly, each section to be inspected (e.g., one or more of the regions 1515a-g, which may be the same as sections used for centerline estimation) may require a dwell time of no less than 6 minutes, with kinematics thresholds set based upon the completeness of the mapped model in the region (e.g., higher velocities on and off the centerline may be permitted once a proper map of the colon region is in place). Where such conditions are not met, or are at risk of not being met, the corresponding region (e.g., one of regions 1515a-g) in the representation 1520b may be highlighted.

As colon length may vary between patients, identification of the regions 1515a-g may be based upon landmarks, or indications by the operators (e.g., operators may have the ability to define the regions themselves as the model is created). In some situations, patients may be classified based upon their physical characteristics to prepare an initial estimate of the colon dimension and corresponding region boundaries 1515a-g, as well as adjustments to the temporal expectations 1525 (e.g., as the same operation may take longer in a patient with a longer colon). Any estimated uncertainty in the colon structure may then be reduced as more information becomes available during the surgical procedure, localization, and mapping. In some embodiments, the system may require proper creation of the colon model within a given region, such that centimeter per second accuracy along the centerline is possible, and only then invite the operator to continue the procedure so that the operator's subsequent relative and residual kinematic instrument motions have the desired resolution.

Consultation with KOLs to refine the kinematic thresholds may be facilitated via review of procedures with GUI elements, such as those described in FIG. 15D. One will appreciate that where the elements are presented during the surgery, timeline indicator 1520f and plot indicator 1520i may be omitted, though they may be included in some instances to describe past portions of the operation.

### Intra-Surgical and Post-Surgical Kinematics Feedback—Projective Relations

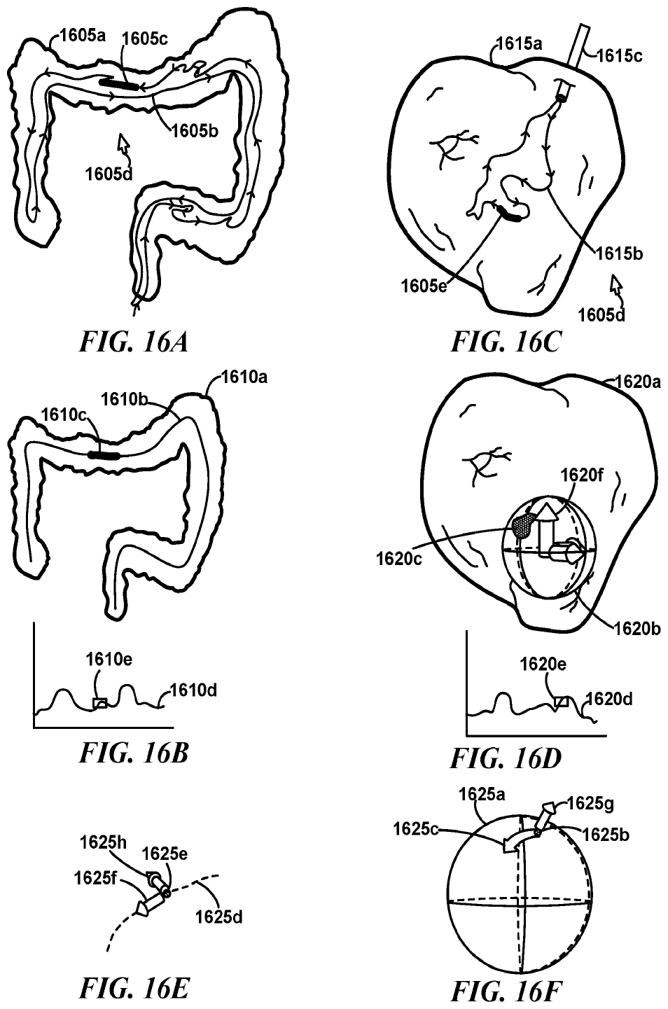

One will appreciate a number of other methods in which the kinematics data derived by the techniques disclosed herein may be presented and used by operators and reviewers. With respect to colonoscopy, FIG. 16A presents a schematic view of a three-dimensional colon model 1605a, with a path graphic 1605b, as may be presented in some embodiments. The model 1605a may be the same as the model derived during localization and mapping, e.g., a TSDF representation or mesh derived therefrom, or may be an idealized colon model (e.g., as prepared by an artist or averaged across a group of known models). The path graphic 1605b may provide an indication of the raw kinematics values for the surgical instrument over the course of a surgery (e.g., the path the camera traveled during the colonoscopy). As shown in FIG. 16B, the same or a different model 1610a may present the corresponding reference geometry, specifically the centerline 1610b relative to the model.

As in the kinematics plot 1520i, a plot 1610d of metrics derived from relative kinematics data, residual kinematics data, or a combination of the two, may be presented to the operator or reviewer. Here, e.g., a region 1605c of the raw pathway, selected, e.g. with a mouse cursor 1605d, may precipitate a corresponding indication of the associated portion 1610c of the centerline (the nearest portions of the centerline to the region 1605c), and a highlighted region 1610e (the metric values derived from the motion in the region 1605c). One will appreciate that selections may occur in reverse, or other orders, e.g., selections of the plot region 1610e may precipitate the highlights 1610c and 1605c. Graphics for each of the residual or relative kinematics may also be presented in colon model 1605a.

For clarity, one will appreciate that the embodiments discussed herein with respect to colonoscopy, and similar tubular regions, such as lungs, esophagi, etc., may readily be performed, mutatis mutandis in other contexts. For example, FIG. 16C is the schematic view of a path graphic 1615b in a cavity model 1615a as may be presented in some embodiments, analogous to the raw kinematics representation in FIG. 16A. Specifically, a raw motion pathway graphic 1615b of an instrument inserted via portal 1615c may be shown relative to the model 1615a. In the same or different model 1620a, the reference geometry, in this example a sphere 1620b, may be shown, as well as a plot 1620d of a metric derived from relative or residual kinematics data based upon the raw pathway 1605e and geometry 1620b. For example, the plot 1620d of FIG. 16D may depict the projected speed of the instrument kinematics upon an axis 1620f, upon a longitude or latitude line of the sphere, etc.

Here, selection, e.g., using a cursor 1605d, of a portion of the plot 1620d, geometry 1620b, or pathway 1615b may result in corresponding highlighting of the other GUI elements. For example, selection of the region 1605e, may highlight the associated portion 1620c of the reference geometry 1620b upon which the selection falls, as well as highlight 1620e the corresponding portion of the plot 1620d. Though not shown, one will appreciate that other of the GUI elements discussed herein, e.g., popup 1520a, timeline 1520f, playback 1520d, thresholds 1520g, 1520j, etc. may likewise be placed in the same GUI as the elements depicted here.

During playback or during the surgery, in some embodiments, the graphical elements may provide a real-time representation of the relative or residual kinematics metrics in relation to the reference geometry. For example, with reference to FIG. 16F, at a given moment during the surgery, or during playback of the surgery, a point 1625b corresponding to the current projected position of the surgical instrument's orientation upon the spherical refence geometry is shown (one will appreciate that each of the spherical examples herein may apply mutatis mutandis to any manifold surface embedded within the Euclidean geometry in which the organ model is presented, e.g., hemispheres, arbitrarily undulating surfaces, etc.). Similarly, as shown in FIG. 16E in the context of a centerline 1625d the current projected position of, e.g., a colonoscope, upon the centerline, either at the present moment in the surgery, or at the present moment in a playback, may be shown via the spherical indication 1625e (or other suitable indications, such as highlighting the portion of the centerline rendering). A length and direction of an arrow in each of the spherical and centerline cases may indicate the present values of the relative kinematics.

For example, the arrow 1625c may indicate the projected direction and amplitude (by its length, color, luminosity, etc.) of the instrument's present projected velocity upon the reference geometry 1625a. Similarly, the arrow 1625f, may indicate the present velocity of the projected velocity upon the centerline 1625d at the current time from the position of indicia 1625e (amplitude again, e.g., being represented by length, color, luminosity, etc.). Similarly, residual kinematics may likewise be presented in the graphical elements. Here, an arrow 1625g orthogonal to the surface of the sphere 1625a at the position of the indication 1625b indicates the velocity component of the instrument orthogonal to the sphere (such component may be useful, e.g., to warn the user when a cauterizer or other instrument too quickly approaches an anatomical artifact). Similarly, the residual kinematics (e.g., movement away from the centerline) in the colonoscope context may be represented by an arrow 1625h, also orthogonal to the centerline 1625d at the point of indicia 1625e.

### Intra-Surgical and Post-Surgical Kinematics Feedback—Graphical Elements

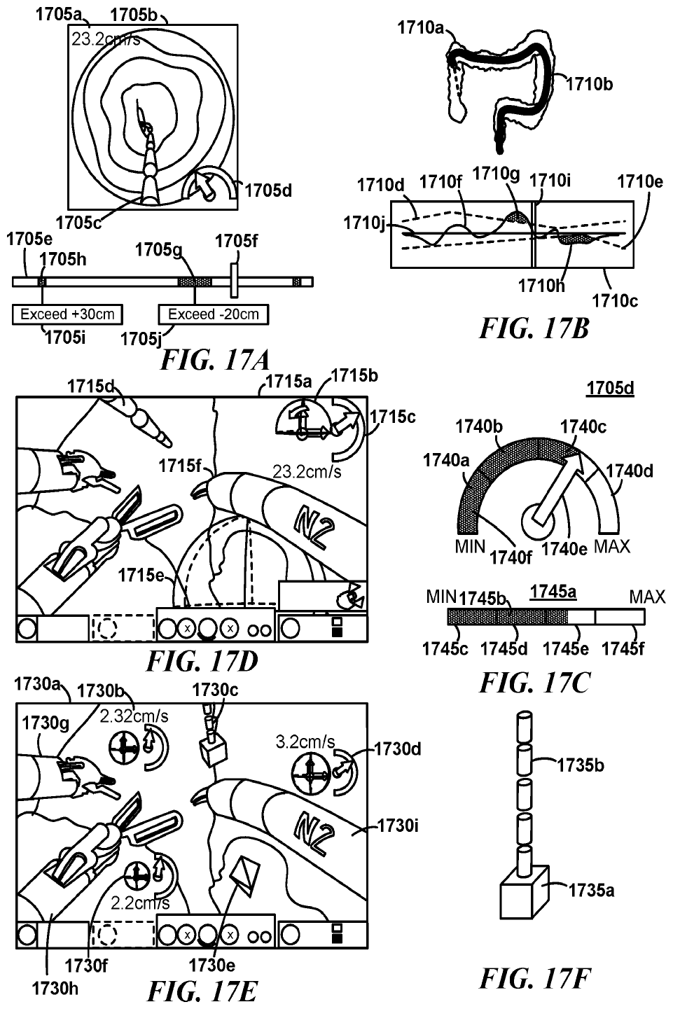

Additional graphical elements which may be used in a GUI during the surgical procedure or afterward during review are shown in FIGS. 17A-F. With respect to FIG. 17A, the GUI may include a playback, or current view, region 1705b for a surgical camera. In addition to presenting the camera's current view, an indication of relative or residual kinematics metrics may be represented. For example, the speed upon the centerline may be shown in an overlay 1705a. A “speedometer” graphic 1705d may help the operator to realize how their motion relates to thresholds, such as the maximum or minimum permissible speed for the spatial and temporal context of the surgery. In some embodiments, during or after the surgery, the element presenting the camera field of view may be supplemented with augmented realty graphic elements. For example, in this example, an augmented reality representation of the centerline 1705c is provided. Here, the user can readily perceive that the camera is above the centerline representation 1705c. During the surgery, such overlays may be provided upon the operator's request, e.g., to provide quick adjustments. In some embodiments, the augmented reality overlay may be translucent so that the operator can still perceive the original camera field of view.

As described previously, where surgeries are being reviewed after their completion, a timeline 1705e may be provided, with an indicator 1705f of the current time in the playback (e.g., the time associated with the currently depicted camera image in the element 1705b). Regions with significant kinematic events may be indicated, e.g., by changes in hue or luminosity upon the timeline 1705e as described herein. As previously described, popups may also be used to annotate the events. In this example, the popup element 1705i indicates that the speed along the centerline in the advancing direction exceeded a desired threshold in the temporal region 1705h and popup 1705j indicates that the velocity threshold was exceeded in the withdrawing direction in the temporal region 1705g. The color of the reference geometry, as reflected in the augmented reality element 1705c may change during these regions, or as thresholds are approached, to warn or inform the operator or reviewer of the possibly undesirable condition.

As shown in FIG. 17B, the representation of the surgical instrument's current position need not be limited to an arrow or other abstract presentation, as computer graphical models of the colon may be used. Here, for example, a model 1710b of a colonoscope is shown in the orientation of the presently depicted frame (e.g., in element 1705b) and relative to the model 1710a of the colon, e.g., the model captured during the procedure, an idealized representation, or a combination of the two. Again, a plot 1710c of derived kinematics metrics 1710f may be shown over the course of the surgery, e.g., with a present time of playback indicated by indications 1710i, and with threshold 1710d and 1710e (here, the baseline 1710j indicating, e.g., a zero velocity along the centerline). As indicated, the thresholds may vary over time and over locations within the organ depending upon the context (e.g., the various spatial and temporal region discussed herein). Regions wherein kinematic events occur, such as those represented by the popups 1705i and 1705j may be shown by corresponding highlights 1710g and 1710h.

For clarity, FIG. 17C is an enlarged view of the reference geometry kinematics “speedometer” graphic 1705d depicted in the GUI of FIG. 17A. In this example, the range of values which the kinematic metric may assume (e.g., speed along the centerline) may be divided into four regions 1740a-d. As described herein, the “minimum” and “maximum” acceptable values may be determined by the spatial and temporal contexts, possibly as informed by KOLs. The current value of the metric may be presented, e.g., with arrow indication 1740e, with a highlighted region 1740f, or with other suitable indications.

For clarity, one will appreciate a myriad number of ways of representing a present kinematics metric value within a context-determined range. As another example, a bar plot 1745a may convey the same information as in the format of the semicircular speedometer 1705d. Again, the plot may be divided into regions 1745c-f with a shaded region 1745b again indicating the present value of the metric.

For clarity in comprehension regarding further variations of the disclosed embodiments, FIG. 17D depicts a robotic surgical procedure interface 1715a (e.g., such as in display 160a). Here, the reference geometry is a hemisphere represented by an augmented reality element 1715e. A corresponding shorthand reference 1715b showing the relative projective kinematic motion of a surgical instrument 1715f of the reference geometry is also provided. A speed of the instrument 1715f's motion (23.2 cm/s) is also overlaid for reference, as well as a speedometer 1715c, e.g., providing similar ranges and thresholds as described above with respect to the indicator 1705d.

One will appreciate that in some surgical procedures there may be more than one reference geometry implicated. For example, in a coloscopy-based removal of a polyp, a reference geometry around the surface of the polyp, and the centerline geometry of the colon may each be used, with their respective relative and residual kinematics datasets collected. Such multiple references may be represented in the operator's GUI, e.g., in the example of FIG. 17D, where an augmented reality guide 1715d is provided to indicate a path along which an instrument is expected to travel, in combination with augmented reality element 1715e surrounding a region of interest.

Indeed, a more expansive collection of such disparate references is shown in the interface 1730a of FIG. 17E. Here, one or more spherical reference geometries is associated with each of the instruments 1730g, 1730h, and 1730i. Accordingly, corresponding shorthand references, and corresponding kinematic metric values and speedometer indicators 1730b, 1730f, and 1730d (corresponding to instruments 1730g, 1730h, and 1730i, respectively) may be presented as overlays, augmented reality elements, etc. Where there are multiple reference geometries available, the operator may cycle through their selection and presentation, e.g., as the operator begins a new surgical task implicating a different one of the geometries than was previously relevant. For example, a marker 1730e may be an abstract geometry created by the operator and inserted into a portion of the field of view as an augmented reality element. Similarly, guide references 1730c may be specifically created or provided to direct an instrument along a preferred approach path. For example, as shown in greater detail in FIG. 17F, some reference geometries may themselves comprise a composite of reference geometries. The depicted guide geometry includes a first geometry 1735b analogous to the centerline geometry, which may be used to guide an instrument to a location associated with the second geometry 1735a. Though the second geometry 1735a shown here is a box, the second geometry 1735a may take on a form suitable for a given location, operator, etc. For example, the geometry may assume the surface contour of an anatomical artifact, a helper geometry for placement of cauterizing and other tools, etc.

### Overview—Image Viability Assessment

As discussed above with respect to block 625b and confirmation 895, various embodiments will examine a sensor's field of view prior to providing the sensor's data to downstream processing operations, such as the localization and mapping operations discussed herein. Where the sensor is a camera, the field of view may be evident from the camera's image data itself. Though discussed herein primarily in the colonoscopy context to facilitate the reader's comprehension, one will appreciate that various of the disclosed embodiments may be applied mutatis mutandis in a variety of contexts, such as esophageal examination, pulmonary and bronchial examination, etc.

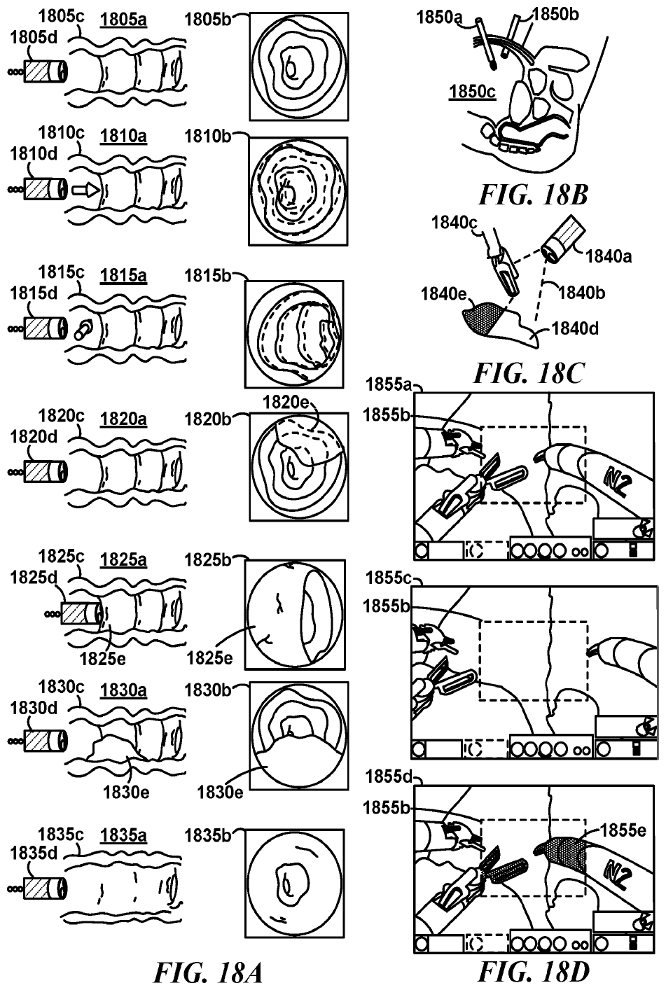

With respect to localization and mapping from colonoscopy images, a number of situations may render the fields of view unsuitable for the downstream processing. Specifically, FIG. 18A provides a schematic collection of various surgical camera states and the corresponding fields of view as may be encountered in the colonoscopy context. In the situation 1805a, the colonoscope 1805d may be in a position and orientation within the colon 1805c such that the resulting camera field of view 1805b is without significant artifacts or obstructions. In such situations, the downstream processing may readily be able to, e.g., infer the location of the colonoscope relative to previously acquired data and to infer accurate depth values as the colonoscope advances or withdraws through the colon 1805c.

However, in the situation 1810a, the colonoscope 1810d has advanced too quickly along the medial axis of the colon 1810c for proper localization. This may result in the camera producing a motion blurred image 1810b. While acceptable motion blur may differ among surgical operators, in general, their tolerance for motion blur may not be the same as that for the downstream processing. For example, the localization system's tolerance may be lower than the operator's. Thus, there may be situations where the resulting motion blur is either not noticeable or not so disrupting as to cause the operator to adjust their workflow, but where this perceived minor blur is in fact disruptive to the downstream processing. Absent notification, the operator may proceed through the procedure, blithely unaware that the processing is “unable” to keep up with the operator's progress. A blurred image, such as image 1810b may challenge many localization algorithms, as it may, e.g., be difficult to distinguish a smooth organ sidewall, viewed statically, from the smooth blur of the image 1810b. For example, application of a SIFT algorithm may produce features quite different from the clearly perceived image 1805b. Thus, from the system's perspective, rapid withdrawal or advance precipitating blur may closely resemble the turning of the camera toward a nearby sidewall. Consequently, erroneous or otherwise improper localization results may follow if downstream processing is permitted, with consequent errors in the further downstream processes, such as mapping and modeling. Similar to the advancing and withdrawing motion blur in the situation 1810a, in the situation 1815a, too quick a lateral motion, or too quick a rotation of the camera 1815d within the colon 1815c may produce a blurred image 1815b.

As another example, while situations 1810a and 1815a produce image-wide blur, localized blur 1820e may also occur, as in the image 1820b of situation 1820a, where the camera 1820d is not moving within the colon 1820c, but fluid has accumulated on the camera lens to produce the localized blur 1820e. In addition to being localized, the blur 1820e is not necessarily associated with a smoothly transitioning vector gradient as in the blur of situations 1810a and 1815a, since the optical properties of the fluid may vary with its density. Thus, localized blur 1820e's presentation may not be consistent, in location, shape, or density, and thus more difficult to identify the frame-wide blur of images 1810b and 1815b (which, indeed, may be discernible via optical flow or frequency analysis).

Even when the image frame is acquired without blur, in some circumstances it may still be unsuitable for downstream processing. For example, in the situation 1825a, the camera 1825d has approached too closely to a colon 1825c sidewall. Consequently, an occluding haustral fold 1825e obscures most of the field of view, resulting in so substantial a portion of the field of view being occluded in the image 1825b that downstream processing will be adversely affected. For example, SIFT features determined upon one small portion of a sidewall are unlikely to differ in a meaningful manner from features determined upon another small portion of the sidewall, thereby mitigating the features' utility for localization.

Similarly, biomass 1830e may so obscure the field of view, as in the image 1830b of situation 1830a, that downstream localization of camera 1830d and mapping of the colon 1830c become infeasible, or at least susceptible to erroneous results. This may be especially true where the biomass is not static, but appears at various locations at various times during the surgical procedure. Should SIFT, or similar, features be derived from the biomass, their application in localization may risk attempting to map a dynamic object to a generally static environment. Thus, a failure to achieve appropriate localization is often itself indicative of some other contextual condition, which may have significance for other machine learning processes (e.g., a biomass recognition and characterization system).

To further complicate matters, one will appreciate that various of the situations discussed herein may occur simultaneously, as when there is both a motion blur and localized blur due to fluid. Thus one will appreciate that the situations 1810a, 1815a, and 1820a are not necessarily mutually exclusive. Accordingly, various of the embodiments disclosed herein may seek to recognize not only the existence of these situations individually, but in combination (e.g., labeling images appropriately as combinations of adverse situations).

For clarity, one will appreciate that not all events affecting downstream processing may need to be recognized by various embodiments disclosed herein. For example, air injection systems in the colonoscope 1835d may readily facilitate inflation of the colon 1835c, as in the situation 1835a, to produce a view 1835b with mostly smooth sidewalls and reduced extension of the haustral folds. However, because such inflation is initiated at the operator's command, some embodiments may mark all images for a period following such modification as being unsuitable for localization. Thus, automatic suitability determination methods, as descried herein, may be sometimes used in combination with other mechanisms to recognize undesirable frames (e.g., operators may manually disable the downstream processing via an interface; encoders may be monitored to recognize motion precipitating blur; software, firmware, or hardware for performing field of view altering operations, such as the inflation in situation 1835a, may cause frames to be marked as unsuitable during their operation's application and for a period thereafter; etc.).

Again, while, to facilitate the reader's understanding, most embodiments herein are disclosed within the colonoscopy context for consistency of reference, one will appreciate that various of the embodiments may be applied mutatis mutandis in other contexts. For example, in the laparoscopic procedure of FIG. 18B, such as a prostatectomy, instruments 1850a and 1850b have been inserted via portals into an interior cavity 1850c. In some procedures, localization of the camera (and perhaps indirectly of other instruments and objects within the field of view) and possibly mapping of the cavity 1850c or structures therein may be desirable. As motion blur, occlusions, fluid blur, etc. may occur in these situations as well, many of the disclosed embodiments may be applied in these contexts with appropriate variation. For example, in FIG. 18C a portion of a laparoscopic camera 1840a's field of view 1840b is occluded by an instrument 1840c, thus resulting in a partially visible portion 1840d and occluded portion 1840e of the tissue. As in the colonoscopy context, recognizing such a situation may be helpful in preventing downstream processing for attempting to reconcile the pixel values associated with instrument 1840c.

Similarly, as shown by the example of FIG. 18D, one will appreciate that many of the embodiments disclosed herein may also be applied upon all, or less than all, of a camera's field of view. For example, whereas localization and mapping during colonoscopy may avail itself of the entirety of the camera's field of view, in some applications, localization and mapping may be of interest only to a particular portion of the field of view. For example, in the image 1855a a region 1855b of a surgical interface has been designated for data acquisition (e.g., where the user wishes to produce a model of a tissue region, excise an organ artifact in the region, recognize a tumor in the region, model the structure of the tumor in the region, etc.). Instruments in this region 1855b, as shown in image 1855a, may complicate or thwart downstream processing operations associated with the data acquisition. Thus, the system may invite the user to retract the instruments from the region 1855b as shown in image 1855c, or the user may do so at their own initiative. Similarly, as will be desired in greater detail herein, in some embodiments, recognition of an non-viable image may precipitate a variety of responsive actions by the system. For example, in the image 1855d, detection that the image within the region 1855b is not viable for downstream processing has precipitated application of a You-Only-Look-Once (YOLO) network (e.g., as described in You Only Look Once: Unified, Real-Time Object Detection, arXiv™:1506.02640, by Joseph Redmon, Santosh Divvala, Ross Girshick, and Ali Farhadi; or, e.g., using methods described in Grad-CAM: Visual Explanations from Deep Networks via Gradient-based Localization, arXiv™:1610.02391, by Ramprasaath R. Selvaraju, Michael Cogswell, Abhishek Das, Ramakrishna Vedantam, Devi Parikh, Dhruv Batra, or, e.g., as described in YOLOv7: Trainable bag-of-freebies sets new state-of-the-art for real-time object detectors, arXiv™:2207.02696, by Chien-Yao Wang, Alexey Bochkovskiy, Hong-Yuan Mark Liao may be used), to produce highlights, such as highlight 1855e, to indicate that the instruments are likely causing the unviable determination. Such highlights may better inform the user when inviting them to retract the instruments from the region 1855b, as shown in image 1855c. Some embodiments may thus incorporate, or anticipate downstream processing, of additional “filtering” options, such as tool recognition and classification, additional environment analysis and processing (e.g., precipitating a change in the surgical procedure, tasks to be performed, priority of operations, etc.).

Naturally, such responsive actions may be taken for the situations in FIG. 18A as well, as when determination that the image is not viable depicts application of a pixel classifier to highlight biomass 1830e, occluding sidewall 1825e, or blur 1820e. Application of a Fourier transform, optical flow algorithm etc. may likewise be triggered for the images 1810b, 1815b and 1820b to determine the nature and location of the blur. Similarly, while viability recognition may occur on a single static image, in some embodiments, classification as non-viable may precipitate processing that considers a sequence of images. For example, detecting a lack of viability in one image may trigger reconsideration of a window of surrounding images, e.g., calculating an optical flow between images in the window, inferring motion (and possible associated causal factors for the lack of viability) in images within the window, etc.

### Example Data Pre-Processing and Post-Processing

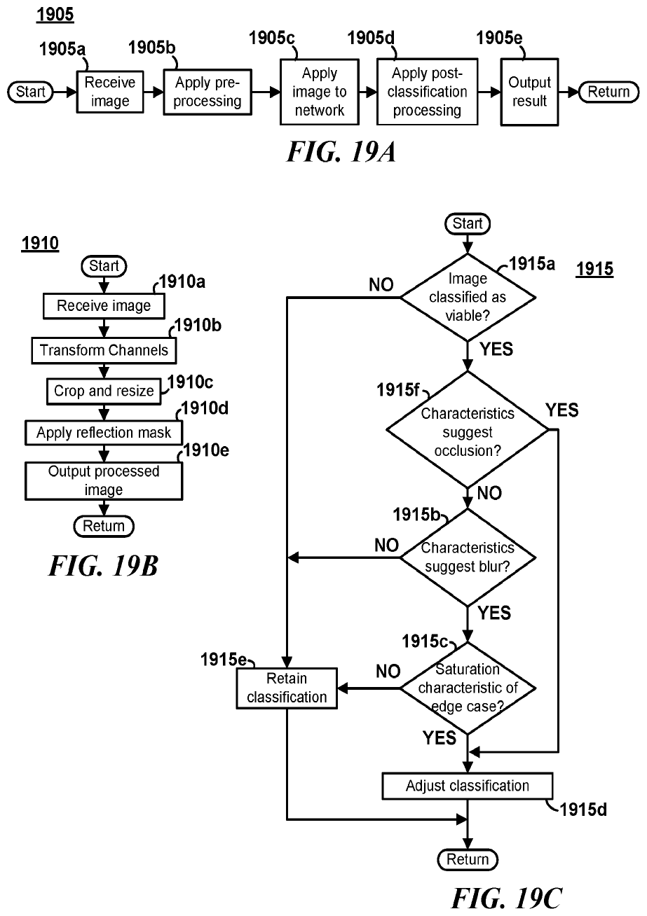

To distinguish viable images from the non-viable images in the situations of FIG. 18A, some embodiments employ various implementations of the general process 1905 shown in FIG. 19A. Specifically, at block 1905a, the system may receive a visual image captured by an intraoperative camera. For example, where the process 1905 is being applied during the procedure itself, the image 1905a may be the most recently acquired image, or the next image in a queue expected to be processed by the downstream operations. However, the process 1905 may also be applied offline, as in post-surgical situations described herein, where one wishes to assess the surgical data after the surgery.

At block 1905b the system may pre-process the original visual image, e.g., cropping the image to appropriate dimensions for input to a neural network, adjusting channels to those expected by the neural network, performing Adaptive Histogram Equalization (CLAHE), applying a Laplacian, etc. as described herein. An example pre-processing process is shown herein with respect to FIG. 19B. Normalizing images via pre-processing may include transforming the image values such that the mean and standard deviation of the image become 0.0 and 1.0, respectively. For example, one may subtract a channel mean from each input channel and then divide the result by the channel standard deviation, as shown in EQN. 7:

$$output [ channel ] = \frac{( input [ channel ] - mean [ channel ] )}{std [ channel ]} \qquad ( 7 )$$

At block 1905c the processed image may be input to one or more neural networks, e.g., a network as described herein. In some embodiments, as described in further detail herein, a preliminary step between blocks 1905b and 1905c may be applied to determine which network of a corpus of networks should be applied to the pre-processed image to assess viability. In some embodiments, following the network's determination, post-classification processing may be applied at block 1905d to produce a final viability determination. For example, various input edge cases may be addressed in the post-classification processing. Process 1915, described in FIG. 19C, for example, provides one example post-classification operation. Once the system determines the final classification value for the image, the result may be output at block 1905e (e.g., for performing the decision of block 625b or the confirmation 895).

Various operations in an example pre-processing process 1910, as may occur at block 1905b, are shown in FIG. 19B. Here, at block 1910a, the system may receive the visual image, as acquired, e.g., by the colonoscope camera, and transform the image's channels appropriately at block 1910b for use by the neural network, or neural networks, as at block 1905c. The image may similarly be cropped and resized for application to the network, or networks, at block 1910c. A reflection mask may be applied at block 1910d. The image may be output at block 1910e, e.g., for consideration by the neural network at block 1905c.

Conversely, processing may be applied in some embodiments following application of the neural network at block 1905c, e.g., to recognize common edge cases to which the neural network, or neural networks, are susceptible to misclassification. For example, some neural networks may incorrectly classify blurred images as valid when those images contain a high number of, or large, reflective highlights (e.g., when a colonoscope shines a light upon an irregularly corrugated surface). Similarly, some occluded images may appear similar to regions with many homogeneous pixel groupings (e.g., a large cavity, darkened aperture, or sidewall). While the neural network may be generally able to recognize the low frequency character of most blurred and occluded two-dimensional images, some situations, such as the presence of many highlights amidst blur, may cause sufficient transitions so as to result in misclassification with relative consistency (e.g., a smoothly contoured series of ridges may resemble the blurred image in these situations, at least insofar as the highlights are similarly placed). Particularly, saturated portions of images resulting from projector light reflected from the surface, which appear blurred or smudged, may consistently precipitate misclassification (such images often being non-informative as a substantial number of saturated pixels similarly affects localization as would a substantial number of obscured pixels).

Thus, in the post-processing at block 1905d, the system may apply a variety of edge case remediations via logic or a supplemental classifier. For example, remediation addressing occlusions and blurred saturation may be accomplished by a process such as process 1915, which first determines if the image was classified as valid at block 1915a, retaining the classification at block 1915e if so, since the process 1915 of FIG. 19C is focused only upon false positives and not false negatives (though one will appreciate similar processes for various false negatives). For images marked as viable by the network or networks, at block 1915f, the system may first consider the image depicts an occlusion misclassification edge case. For example, hue thresholding, Euler counts, flood-filling, frequency analysis, SIFT feature analysis, etc., may all be employed to determine if the image depicts an occlusion edge case. If an occlusion is found to be present, this post-classification logic may adjust the classification at block 1915d.

At block, 1915b the system may consider whether the image contains blur. For example, while application of a neural network at block 1905c may be suitable for recognizing a wide variety of blurs, such as motion blur, localized blur, etc. direct analysis of the image with traditional processing techniques may reveal the presence of blur in those edge cases where a high number of reflections has precipitated a false positive. Thus, blocks 1915b and 1915c may operate together to determine that the image depicts the contemplated edge case. For example the frequency content of the image in the presence of highlights may provide a sufficiently consistent and unique profile for recognition using a traditional binary classifier, such a support vector machine (SVM), logistic regression classifier, etc. While hard threshold values may be used in some embodiments based upon inspection, one will appreciate that a classifier, such as an SVM, may be readily trained to perform the operations of blocks 1915b and 1915c, distinguishing between genuinely blurred images and blurred images with a requisite number of highlights. For clarity, such an SVM may have its own preprocessing steps applied to the image received at block 1905a, and such preprocessing steps may be the same or different as those at block 1905b. Rather than the number of reflections, block 1915c may instead assess the portion of the image occupied by highly saturated pixels, as by one or more reflections. Where the image meets the edge case conditions, the classification may be accordingly adjusted at block 1915d. One will appreciate that such exclusionary operations may also be applied to eliminated frames before their consideration by the network (e.g., an image of nothing but black pixels clearly shouldn't even have the opportunity for classification as valid by a network). Edge case consideration after viability classification processing, however, may be more suitable for edge cases which affect less than all, or inconsistently affect, portions of the image (such as reflection dispersals and high saturation regions).

Though the example process 1915 depicts an occlusion assessment and then a blur and saturation assessment, one will appreciate variations based upon this disclosure wherein each edge case is separately considered, as well as additional edge cases are considered. Accordingly, the operations of block 1905d may include only the blocks 1915f, 1915b, 1915c rather than the depicted sequence (e.g., saturation alone may be assessed without considering blur). The choice of such logic may be identified in parallel with training of the one or more neural networks, as the logic and corresponding thresholds may be selected so as to improve the overall classification results during validation. Again, for clarity, some embodiments forego process 1915, and indeed, forego all of post-processing at block 1905d, to instead rely only upon the classification determined by the one or more networks at block 1905c. Such approaches may be suitable where the network was exposed to an adequate variety of training samples so as to account for the desired edge cases.

### Example Viability Assessment Neural Networks

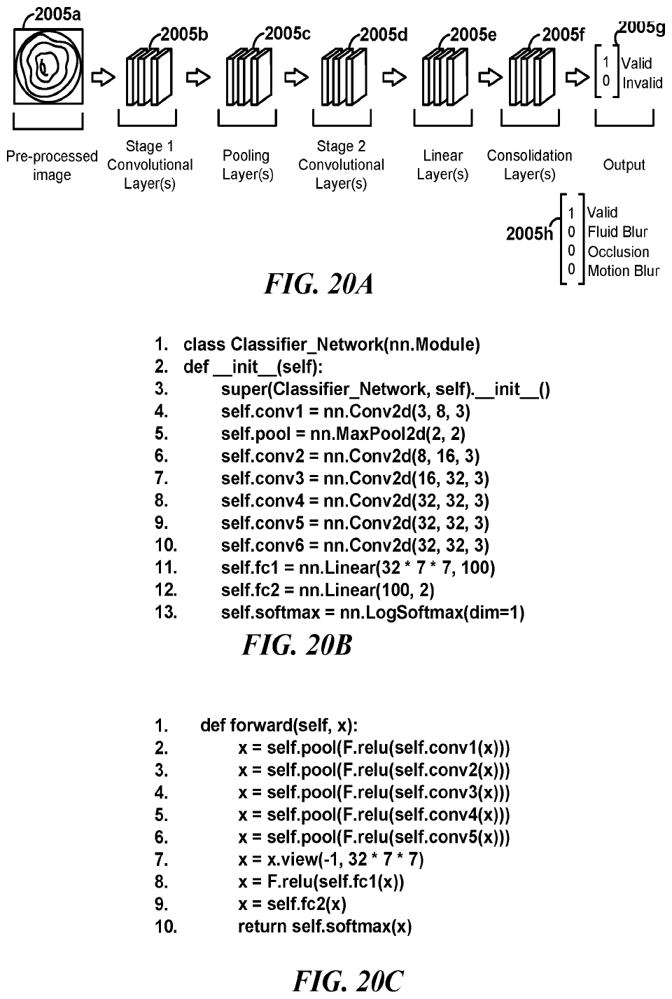

FIG. 20A is a block diagram illustrating an example neural network architecture as may be used in some embodiments to distinguish viable and non-viable images (e.g., at block 1905c). In this example architecture pipeline, the system provides the processed image 2005a (e.g., following the operations of block 1905b and process 1910) to a first stage of one or more convolutional layers 2005b. The first stage of one or more convolution layers 2005b is itself coupled to one or more pooling layers 2005c, which may themselves be coupled with a second stage of one or more convolutional layers 2005d. Linear 2005e and consolidation layers 2005f may then follow to produce a final output classification as viable or non-viable. While, in some embodiments, the final output may be a binary classification, e.g., “valid for downstream processing” or “non-viable for downstream processing”, as indicated by output 2005g, some embodiments may instead tease apart the different failure states, e.g., those described in FIG. 18A, providing an output with multiple classes, each class other than the viable class, indicating a reason for the frame's being classified as non-viable 2005h. Having the additional knowledge of the nature of and reason for the failure may better inform surrounding processes and review, including the downstream processing. Naturally, the labeling of the training and validation data will be changed when a multi-class output is used.

While one will appreciate a variety of methods for implementing the network structure of FIG. 20A, as an example to facilitate understanding, FIG. 20B provides a partial code listing for creating an example implementation of the network topology depicted in FIG. 20A. In this example, written in the Python™ language and using the Torch™ machine learning library, the class Classifier_Network extends the Torch™ class Module (lines 1 and 3), creating an example implementation of the structure appearing in FIG. 20A in the initialization function of lines 4-13.

Specifically, in this example, line 4 corresponds to a first stage of one or more convolutional layers 2005b, line 5 corresponds to the one or more pooling layers 2005c, and lines 6-10 correspond to the second stage of one or more convolutional layers 2005d. lines 11-12 then depict an example implementation of the linear layers 2005e before connecting with the softmax layer at line 13, corresponding to consolidation layers 2005f, to output the result. Here, line 13 indicates a single dimension for a binary result, but a multi-class output may be produced by increasing the dimensions.

For additional clarity, FIG. 20C is a partial code listing for performing forward propagation upon the example network implementation of FIG. 20B. Specifically, continuing the Classifier_Network class definition begun in the listing of FIG. 20B, here line 1 specifies the reimplementation of the forward propagation function of the nn.Module, while lines 2-6 then specify the connection between the convolutional layers as rectified Linear Units (ReLU).

Similarly, line 8 indicates that the linear layers of line 11 in FIG. 20B are connected via a ReLU torch.nn.functional “F” and line 8 indicates a direct passthrough output for the linear layer of line 12 in FIG. 20B. Finally, at line 10, the result of the softmax upon the linear result may be output. As the softmax output presents a value between 0 and 1, naturally it is suitable for a binary classifier as in the example outputs 2005g between viability and non-viability alone. Where a multiclass network is used, one will appreciate adjustment of the connections accordingly. Thus, the network may learn and extract semantic information from the visual image suitable for determining the image's viability for either depth estimation, and consequent camera localization for constructing the entire three-dimensional representation of the surface. The network, in recognizing the absence of suitable cues for those operations (e.g., an appropriate pattern or number of SIFT features), may thus recognize that the frame is not valid for these operations.

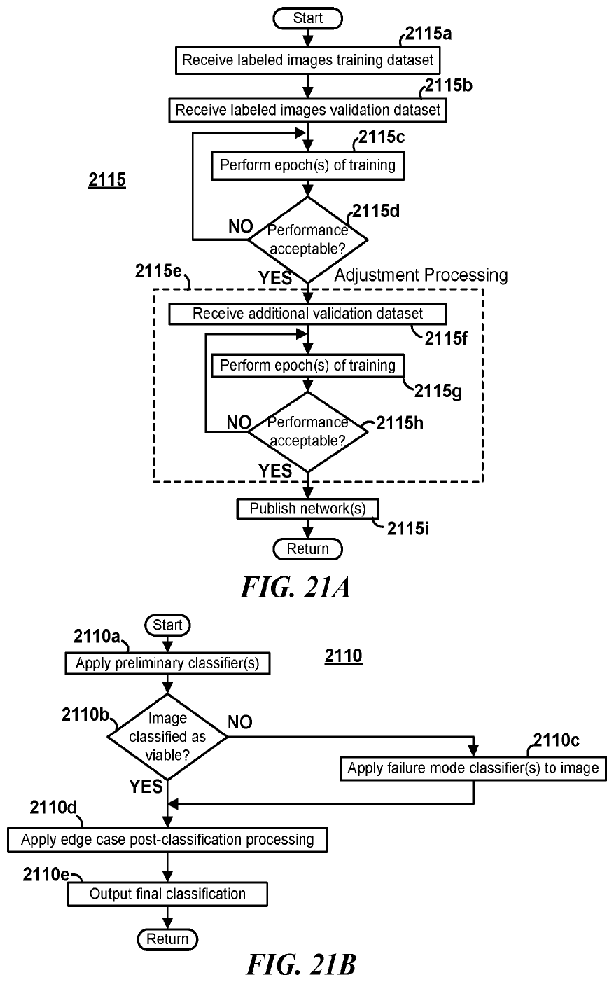

FIG. 21A is a flow diagram illustrating various operations in a network training and validation process 2115, as may be implemented in some embodiments. Specifically, at block 2115a, the training system may receive a training set of labeled images, as well as images with labels identified for validation at block 2115b. Here, creation of the training dataset may involve applying images acquired with a colonoscope, bronchoscope, etc. to the downstream processing, validating whether the downstream processing results were within or outside acceptable tolerances, and then labeling the images accordingly. Thus, training and validation datasets may be created by providing a corpus of real-world images, at least some of which were believed to exhibit the various phenomenon of FIG. 18A, to the downstream pipeline as would occur in normal in-situ processing. Images producing results in the downstream processing within tolerance may be labeled “valid”, while images producing results outside the tolerance may be labeled “invalid” (or the appropriate class for a multiclass non-viable labels, e.g., in accordance with the adverse situations of FIG. 18A). Where the downstream processing is, e.g., localization and mapping, the results for the images may be compared to ground truth results and only localization poses within a maximum distance from the true pose labeled as “valid”.

Epochs of training may be performed with such data upon the neural network at blocks 2115c and 2115d until the network's performance is found to be acceptable at block 2115d. For viable and non-viable binary classifications (e.g., as in output 2005g), binary cross entropy over a ground truth labeled dataset may be used to assess the loss. For multiclass outputs (e.g., as in output 2005h) multiclass cross entropy over a ground truth labeled dataset may be used.

In some embodiments, the satisfactorily performing network from block 2115d may be provided directly to block 2115i for publication. However, in some embodiments, a portion of the training dataset, shown here provided at block 2115f, may be withheld for further validation and adjustments in a second round of training 2115e. While iterating through the blocks 2115g and 2115h, edge cases may be detected and appropriate post-classification processing operations preprepared (e.g., determining the parameters for detecting the blur and reflections edge case of FIG. 19C). Once performing acceptably, and the desired edge cases and their parameters properly identified, the network may be published for in-situ use at block 2115i.

Based upon this disclosure, one will recognize a variety of network architectures and corresponding training methods which may be suitable for distinguish viable images from the adverse situations 1810a, 1815a, 1820a, 1825a, and 1830a depicted in FIG. 18A. Indeed, one may train multiple neural networks, or other ensembles of classifiers, to provide more robust classifications or redundancy-based verification, e.g., taking the majority vote, or weighted vote based upon the validation performance of the constituent networks. Some embodiments may thus employ the topology of FIG. 20A alongside implementations of the Vision Transformers (ViT) network, the mobile ViT network, mobilenet, etc. Again, one will appreciate that while particular suitable architectures and training methods are presented herein, one can vary the architecture structure and the training methods considerably, while still retaining the same functional effect.

In some embodiments, rather than apply classifiers in parallel, at least some of the classifiers may be applied in serial. For example, a first set of one or more classifiers may be trained to recognize viability or non-viability generally, while a second set of one or more classifiers may be trained to recognize a class (e.g., one of the adverse situations of FIG. 18A) of a non-viable image. When deployed, the second set may be used to determine the nature of the non-viable classification. Indeed, because of their different functions, the two sets of classifiers may assume different architectures, as when the first classifier is not a neural network, but a simple binary classifier, such as an SVM or logistic regression classifier operating upon a Principal Component representation of the visual image, while the second classifier is a neural network with convolutional layers.

For clarity, FIG. 21B is a flow diagram illustrating various operations in a serial multi-classifier classification process 2110, as may be implemented in some embodiments. At block 2110a, the one or more classifiers in the first set of classifiers (e.g., the SVM or logistic regression classifier described above) may provide an initial indication of viability or non-viability. Where an SVM is applied, in the relatively controlled context of colonoscopy, gray scaling the original image, applying the Laplacian, and then applying Principal Component Analysis may sufficed to produce features adequately separated for classification using an SVM with a radial basis function kernel. Where the first set of one or more classifiers determines a high probability viability classification, the system may simply output the valid classification, proceeding directly to block 2110e, though in the depicted embodiment, edge case detection and classification adjustment as described herein may be first performed at block 2110d. Thus, where ensembles of classifiers are applied, consensus among the classifiers may determine the viability classification. However, as discussed, while some embodiments apply combinations of classifiers, e.g., in an ensemble, and substantial pre-processing operations (e.g., application of the Laplacian), in many embodiments it suffices to apply only one neural network, e.g., as discussed with respect to FIGS. 20A-C, upon an image with minimal preprocessing (e.g., that presented in the EQN. 7) to achieve sufficiently accurate filtering results.

Where the first set of one or more classifiers instead classifies the image as non-viable at block 2110b, the system may provide the image to the second set of failure mode classifiers at block 2110c. In some embodiments, the second set of one or more failure mode classifiers may also consider the particular results from the first classifiers to better facilitate classification (e.g., not only the binary non-viable or viable result of a logistic regression classifier, but the actual numerical value returned by the classifier).

Following processing by the failure modes classifiers 2110c, the final classification result may be provided at block 2110e, though again, in this embodiment, edge case processing is first performed at block 2110d. For example, edge cases between the may precipitate misclassification of one adverse situations as being another of the adverse situations (e.g., a large fluid blur covering most of the field of view may be confused with motion blur absent consideration of encoder motion, a frequency analysis of the original image, etc.).

One will also appreciate that the training process 2115 may be used for training both the first and second sets of classifiers in the process 2110. For clarity, given a corpus of images, tolerance verification of the downstream processing may first be used to label the images as viable and non-viable. This dataset may then be used for training and validating the first set of classifiers, used at block 2110a, in accordance with the process 2115. A second dataset may then be created by manually inspecting and labeling the non-viable labeled images of the first dataset with their respective classes (e.g., the adverse situations of FIG. 18A). This second dataset may then be used for training and validating the second set of classifiers used at block 2110c, again in accordance with the training process 2115. In training each of the first and second sets of classifiers, the edge case verification from the operations in block 2115e (for each set of classifiers) may be used in the post-classification processing of block 2110d.

### Example Surgical Assessment Including Visibility Record

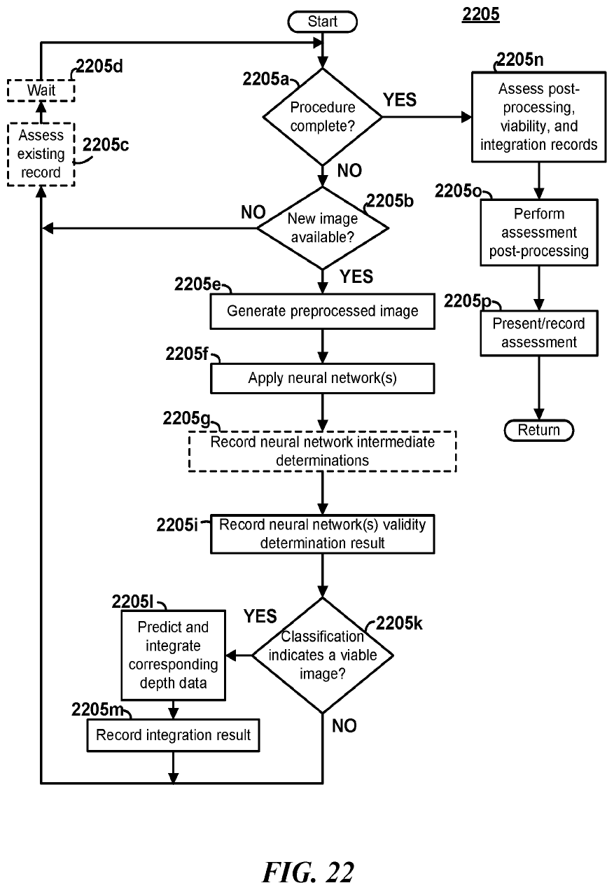

FIG. 22 is a flow diagram illustrating various operations in an example process 2205 for inferring surgical performance data from viable and non-viable image classification results, as may be implemented in some embodiments. Specifically, in addition to their facilitating efficient downstream processing, application of the image viability classification systems and methods disclosed herein may also enable novel types of surgical operator assessment and feedback. Here, until the procedure is found to be complete at block 2205a, the system may consider newly arrived images at block 2205b (e.g., those arriving directly from the surgical camera during the procedure, the oldest image of images from the camera queued for processing, etc.). Where a new image is available at block 2205b, the system may prepare a pre-processed version of the image at block 2205e (e.g., applying the operations of process 1910) for consideration by the one or more neural networks (e.g., the first and second sets of networks discussed in FIGS. 21A and 21B, the network of FIG. 20A, etc.) at block 2205f.

In some implementations of process 2205, at block 2205g the system may record not only the neural network's final classification, but also the various intermediate results. For example, the numerical output, and not simply the final classification, of an SVM, or logistic regression classifier, may be recorded. Similarly, the individual weighted votes for networks in an ensemble configuration, the numerical value of an non-viable classification in a serial configuration, etc. may be recorded. The results of post-classification processing, such as edge case handling, may also be recorded at block 2205g.

Where the classification indicates an image frame suitable for use in downstream processing, here, localization and associated mapping, the system may transition from block 2205k to block 2205l to predict the placement and integration of the derived depth data. The results of this integration may likewise be recorded at block 2205m (e.g., the determined pose at localization, as large spatial distances between successive successful pose determinations)

While assessment of the recorded data may occur following completion of the procedure at block 2205a, in some embodiments intermediate assessments during the course of the procedure may likewise be performed at block 2205c. Such intermediate assessments may be particularly suitable where the surgical operation can be conceived of as a series of discrete tasks. Thus, the system may assess the surgeon's performance during and after a given task, and may provide real-time comparisons to other surgeon's performing the same or similar tasks. As indicated by the block 2205d, one will appreciate that there may be regular periods during which there are no new images and so processing may pause.

When the procedure completes, at block 2205n the system may review the records acquired at blocks 2205g, 2205i, and 2205m, the results of which may be presented at block 2205p. The assessment at block 2205o may consider the numbers of viable and non-viable images throughout the entire procedure and at specific tasks. An increased number of non-viable frames during tasks where proper fields of view are critical (e.g., polyp inspection) may precipitate lower assessments than if the same number or percentage of non-viable frames occurred in less sensitive tasks (e.g., transit to a tumor).

In addition to the simple occurrence count, patterns of non-viable results may also provide information regarding the surgeon's behavior and the context of the surgery. For example, increased numbers of non-viable images during a particular task, which manifests itself over a large corpus of surgeries by different practitioners, may indicate that some aspect of the procedure, the organ, etc., consistently produces an inimical field of view at that location (rather than any given surgeon's actions being the cause for the non-viable frames). Conversely, situations where a task generally produces few non-viable images for most surgeons, but consistently presents non-viable images for a given surgeon, may suggest that the surgeon's performance of that task deviates in an undesirable manner from the methodology of the surgeon's peers. Localization and mapping may be adjusted accordingly or feedback may be provided to the deviating surgeon.

### Example Visibility Network Real-Time Intraoperative Feedback

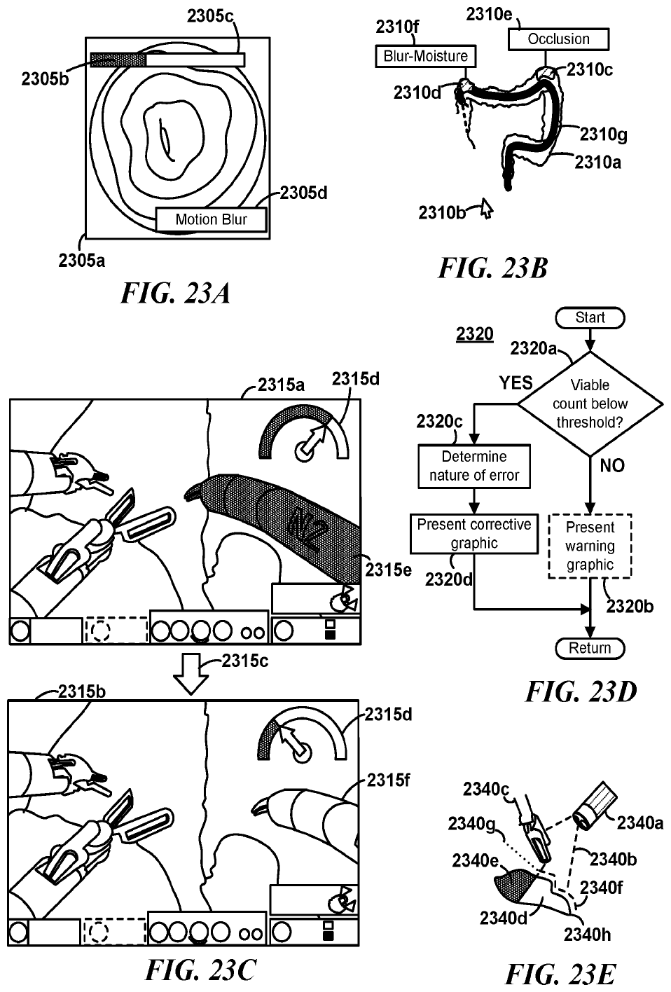

Application of the classifiers described herein may proceed so quickly that their results may be used in real-time not only to avoid improper application of the downstream processing, but also to warn the surgical team that the current field of view is not proper for downstream operations. One will recognize the value of the various feedback methods disclosed herein, regardless of the particular manner in which an image was determined to be viable or non-viable. For example, FIG. 23A is a schematic visual image GUI element 2305a as may be presented in a GUI to surgical team members, e.g., on one or more of the displays 125, 150, or 160a during a surgical operation, or upon a display depicting playback of a recorded surgical operation. Specifically, a first indicator 2305c may inform the surgical team or reviewer regarding how many of the past video frames have been classified as invalid (e.g., if the surgical camera's framerate was 25 frames per second, first indicator 2305c may show a percentage of the past 125 acquired images classified as non-viable). Though shown here as a bar with a solid region 2305b (e.g., indicating the percentage of frames classified as non-viable) one will appreciate that numerical values, dials, etc. may be used instead. Thus, rather than a raw number of non-viable classified images, the indicator 2305c may instead reflect a scaled or mapped value for the number of non-viable images. For example, the indicator 2305c may indicate values in a range from 0 to 1, 0 indicating that the number of non-viable images in the window is entirely acceptable for the current state of operation, whereas 1 indicates that the number of non-viable images is unacceptable. Such a mapping may facilitate adjustment of the feedback to the user in accordance with the current surgery context (e.g., more non-viable frames may be acceptable after mapping reaches an equilibrium state, during non-sensitive portions of the surgical procedure, etc.). For example, an occasional non-viable image in a well-travelled and already well-mapped region of the colon may not warrant the operator's attention. In contrast, when the colonoscope has entered a new, unmapped region of the colon, or a region excepted to include sensitive information (e.g., a tumor or polyp), the same number of non-viable images may have more dire consequences for the downstream processing, and so the need to inform the surgical team may be greater. Thus, surgical context may scale the value appearing in the indicator in some embodiments.

While some embodiments simply notify the surgical team of the existence of non-viable frames, in some embodiments the element 2305a (or other portion of the GUI) may include an indicator 2305d providing guidance as to why the system believes one or more images (e.g., most recently captured image) were non-viable. For example, some operators, unused to operating with the assistance of a digital system, may proceed too rapidly through the colon for the system to maintain adequate localization or modeling. Such undesirable blur may present the warning in the indicator 2305d that the user's actions are producing non-viable images, specifically as a consequence of the user's blur precipitating advance or withdrawal.

In some embodiments, as shown in FIG. 23B, the GUI presented to the surgical team (e.g., on one or more of the displays 125, 150, or 160a) or upon a display to a playback reviewer, may depict the mapping results at a current moment in time, so as to inform the surgical team or reviewer of locations in the model that may have been affected by non-viable images. The GUI element may also include information for how to confirm that no adverse consequences followed from the non-viable images (e.g., by acknowledging and removing the warning), or how to repair the three-dimensional model (e.g., by revisiting a region of the patient interior corresponding to the portion of the model affected by the non-viable frames). Here, the existing model 2310a (e.g., a triangulated textured mesh derived from the TSDF representation) is rendered as well as a representation 2310g (such as an artist's three-dimensional model of a colonoscope) of the current position of the colonoscope. Regions of the model 2310d and 2310c are marked (e.g., with highlighted edges, vertices, changes in texture, etc.) to notify the team or reviewer that during data acquisition at those locations, non-viable images were encountered. While some embodiments may simply indicate the existence of non-viable images and invite the team to revisit the area merely upon that basis, as shown here, billboards 2310f and 2310e indicate the image's non-viability classification (or a majority classification where a sequence of images were found to be non-viable), providing the team with context for returning and correcting the issue. Selecting billboards 2310f and 2310e or regions 2310d and 2310c of the model, e.g., with cursor 2310b, may present additional relevant information, such as the time, colonoscope orientation, and other context of the event.

As shown in FIG. 23C, some embodiments may combine the feedback regarding the recent number of non-viable images with corrective guidance. In this example, at a first instance in time, the GUI image 2315a indicates via indicator 2315d, that a substantial number of recent frames have been classified as non-viable (e.g., analogous to the linear representation of indicator 2305c). In the present context of the surgery, the number of non-viable images was sufficiently high in number as to trigger the system's application of a YOLO network to the field of view. Here, the network is trained to recognize surgical instruments, and thus, highlighting 2315e indicates that one of the surgical instruments has prematurely occluded the field of view, precipitating non-viable images for downstream processing (e.g., preventing adequate model creation for the regions of interest or before proper camera localization could be performed). After 2315c the user has responsively adjusted the position of the instrument 2315e, and viable images begin to accumulate, then, as shown in the GUI state 2315b, the non-viable frame indicator 2315d may begin to go down, and the system may cease application of the YOLO network as well as highlighting of the previously offending instrument 2315f.

FIG. 23D clarifies an example of this feedback behavior with a schematic process representation 2320. Specifically, in this example, if the number of the non-viable images has become critical at block 2320a, then at block 2320c the system may seek to determine the nature of the error and present a corrective graphic at block 2320d (e.g., the highlight over the instrument 2315e following application of a YOLO network). In some embodiments, the acceptable percentage of invalid frames may change with different procedures (e.g., an inspection procedure requiring fewer invalid frames than a simple excision procedure), at different times or locations in the same procedure (e.g., fewer invalid frames during sensitive portions of the operation in the vicinity of a tumor, or during mapping, but not when exiting the anus), or during different tasks in the procedure (e.g., “initial mapping and orientation” may be itself a task in the procedure requiring fewer invalid frames than a purely mechanical excision task).

Even where the non-viable count is not yet above a threshold, a preventative warning at block 2320b may be appropriate (e.g., if the number of non-viable frames has been slowly increasing following an action, such as application of an irrigation device, the system may call attention to the temporal correlation with a warning graphic, particularly if the non-viable images are classified as depicting fluid blur).

Rather than a single threshold one will appreciate that one or more ranges may be applied at block 2320a depending upon the surgical context. For example, when there are no non-viable images or only a handful of incidental non-viable images, the system may take no action. However, if there is a number of non-viable images below the threshold of block 2320a, but associated with a growing trend (e.g., in each successive 100 millisecond window, the number of non-viable images is increasing), then the graphic of block 2320b may be presented. In some embodiments, the nature of the increasing number of invalidity may be investigated. If the frames, e.g., were found to result from motion blur, then the warning graphic of block 2320b and ultimately 2320d may each invite the surgical operator to reduce their speed so as to reduce the resultant blur.

FIG. 23E is a schematic illustration of a surgical tool occluding a portion of a surgical camera's field of view, as may occur in some embodiments, similar to the situation of FIG. 18C and FIG. 23C. In some embodiments, the GUI depicted on one of displays 125, 150, or 160a or on a desktop display during playback review, may translucently overlay or substitute a depiction of the presently captured three-dimensional model within the current field of view as an augmented reality element. In these situations, the portion of the model rendered in the GUI may be adjusted or supplemented to indicate regions of inadequate coverage, regions without any coverage, etc. Here, in FIG. 23E, as in FIG. 18C, an obstruction (here, instrument 2340c, though fluid blur may be likewise identified) may obstruct a portion of the camera 2340a's field of view 2340b of anatomical artifact 2340h, creating a visible portion 2340d of the artifact and a not readily visible portion 2340e. Here, the presently created model is overlaid as an augmented reality element upon the GUI field of view, such that the first portion 2340f of the data-derived model is presented to the user. However, a second portion 2340g of the augmented reality element, may indicate that the field of view is inadequate (e.g., based upon the YOLO results discussed in FIG. 23C), e.g., by being differently textured or rendered than the portion 2340f. Detection of an non-viable image during capture may trigger such overlays and adjustments to model renderings. For example, the unseen portion 2340e would, in normal circumstances, be treated by the localization and mapping process as a naturally occluded portion of the patient interior, as by, e.g., a haustral fold distant from the camera, a curvature in the organ sidewall, etc. Here, however, because the video image frame has been recognized as non-viable by the methods disclosed herein, the occlusion may instead be processed as an undesirable feature to be further investigated (e.g., via application of the YOLO network). One will appreciate a number of methods for rendering the augmented reality portions 2340f and 2340g, e.g., as a textured mesh, or as a two-dimensional billboard aligned in the plane of the camera's field of view within the GUI rendering pipeline.

### Example Post-Surgical Assessment Interface

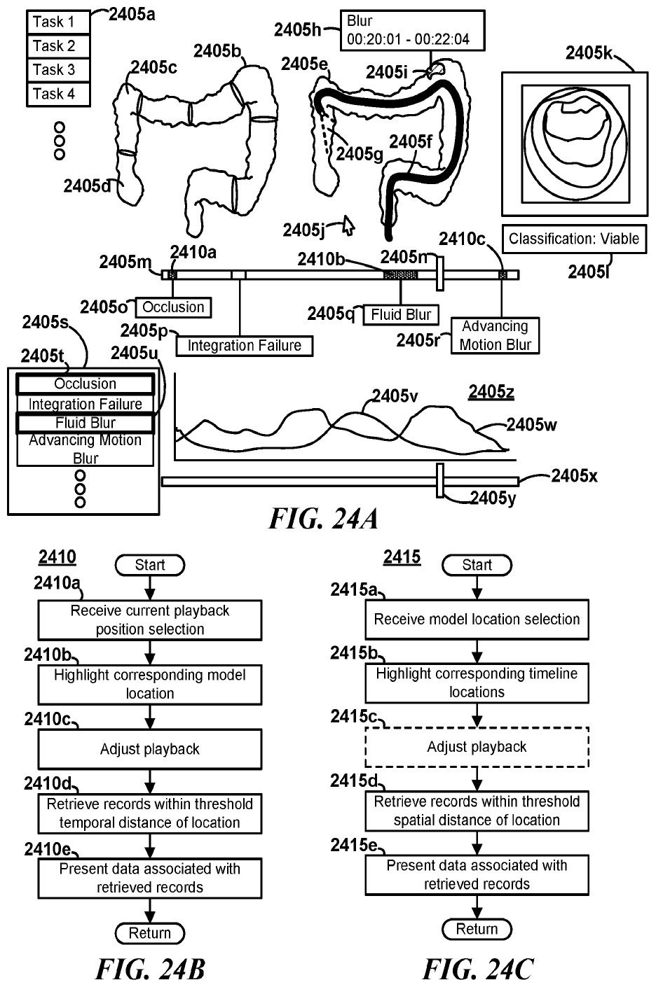

FIG. 24A is a schematic illustration of elements in a GUI for assessing surgical performance based upon a record of image viability data, as may be implemented in some embodiments. For example the GUI elements shown in FIG. 24A may be presented at block 2205p (or at block 2205p, if real-time review is desired, e.g., for a previously completed task). Here, a linear timeline 2405m, with a current playback position indicator 2405n, provides the user with a vehicle for quickly reviewing the captured data. As playback proceeds, the indicator 2405n may advance along the timeline 2405m. During playback, a camera playback region 2405k may depict the visual field at the current time in playback for the surgical camera whose images were assessed for viability. Similarly, the classification element 24051 may indicate the viability classification of a currently depicted frame. YOLO results and other overlays, as they were presented during the surgery, may also be presented in playback region 2405k, e.g., so that the reviewer appreciates what feedback previously provided to the surgical team.

The GUI may also present a representation of the captured depth data in model 2405e, which may include a representation of the camera's position 2405f at the current point in the playback (the model 2405e may be an artist's rendition, the model created during mapping for the surgery, or a combination of the two). Here the field of view 2405g corresponding to the visual field of view in the playback region 2405k may likewise be shown.

Throughout the entirety of playback or at the current position in playback, the system may indicate the regions in the model affected by non-viable images or where non-viable images were encountered. For example, the pop-up 2405h here indicates that images classified as depicting blur were encountered in the region 2405i. Pop-up 2405h likewise includes a time range indicating that the blur was encountered approximately 20 minutes into the surgery and lasted for approximately 2 minutes and three seconds (as determined, e.g., by the range of blur-classified images with less than a threshold number of successive viable-classified images within the range). By clicking on the popup, e.g., with cursor 2405j, the user may direct the system to begin playback at, e.g., the first invalid classified frame associated with the popup 2405h and region 2405i (some embodiments may begin playback at a time preceding the selected interval to provide context leading to a non-viable image classification).

Just as markers, such as popup 2405h, may be used to indicate where non-viable images were encountered in the spatial context of the model 2405e, markers may likewise be provided to indicate temporal locations of interest. Here, e.g., the markers 2405o, 2405p, 2405q, 2405r indicate times along the timeline 2405m when sequences of various one or more non-viable classified images occurred. For example, the marker 2405o may indicate that non-viable images associated with an occlusion may have been encountered at the corresponding time. Marker 2405p indicates that the captured depth values failed to integrate properly, an event which may occur even when frames were classified (perhaps mistakenly) as valid. Here, the presence of an integration failure for images which were classified as valid may suggest that a new, previously unencountered circumstance, is precipitating non-viable images (e.g., a situation unique to the operation and not represented in the situations of FIG. 18A, as when a surgeon has an idiosyncratic manner of moving or placing a surgical instrument). Consequently, such failures may be used to identify images and new labels for future neural network training rounds (e.g., the labels for the frames associated with the marker 2405p would be labeled with a new class for the surgeon's idiosyncratic behavior).

Similarly, the markers 2405q and 2405r indicate times associated with the acquisition of images classified as containing blur. Rather than include any indication of the causes for the non-viable images in the marker, in some embodiments, such as those with neural networks employing a binary output 2005g, the markers 2405o, 2405p, 2405q, 2405r and 2405o may simply indicate that the image was found to be non-viable. Similarly, just as the indication 2405i may correspond to multiple non-viable image classes, the timelines markers may include regions, such as regions 2410a, 2410b, and 2410c indicating the successive number of frames susceptible to invalid classifications. While not all of the images in the regions 2410a, 2410b, and 2410c may share the same non-viability classification, the regions may be assigned a single, continuous classification, as here, where each instance within the successive number of other-classified images is below a threshold (e.g., less than 15 successive other-classified images may be ignored, for a 45 frames per second capture rate).

Indeed, in some embodiments, the timeline 2405m itself vary in hue, luminosity, intensity, etc. in correspondence with a sliding window average of viable and non-viable classifications (in some embodiments, where there are multiple non-viable classes, each class may receive a unique hue or texture). For example, the brightest value in the range may be used when all the images within the sliding window were classified as viable, and the darkest value used when all the images within the sliding window were classified as invalid.

As mentioned, some surgical procedures may be readily divisible into recognizable “tasks” or relatively discrete groups of actions within the procedure (e.g., “advance to excision site”, “excision,” “cauterization,” “post-cauterization inspection,” “withdrawal,” etc.). Selecting the appropriate task in the list 2405a may result in playback (e.g., updating the image in the region 2405k, changing the location of position indicator 2405n, etc.) starting form that task's first associate image frame. In some embodiments, a captured model or a reference model 2405b may be divided into regions. Here, for example, the model is divided into seven regions, including the regions 2405c and 2405d. Just as selecting a task from list 2405s began playback in that region, so may selecting a region, e.g., using cursor 2405j, begin playback at the first frame associated with that region (e.g., at the user's indication, only withdrawal or advancing encounters may be considered, the two being distinguished by the time between intervening encounters with the region, as well as the starting location of the encounter). One will appreciate that the networks herein, such as that of FIG. 20B, may be readily configured to include location and other contextual information within their training and inference inputs. That is, the system may not only consider the pixel values of the image in assessing validity or invalidity of the image frame, but the present location of the colonoscope in one of the regions of the model 2405b, the task from the list 2405a which is being performed during the image capture, etc.

In some GUI implementations, the current surgical performance may be compared to other performances, e.g., by the same or different surgical operators. In some embodiments, e.g., those implementing a binary classifier 2005g, the comparison may simply be between the number of non-viable and viable images across the entirety of each surgery, within particular tasks, as well as the frequency of non-viable intervals in the tasks in the surgery, or the number of successive non-viable images. Plotting the incidences over time may help the user to recognize patterns in their behavior precipitating non-viable images, as well as portions of the surgery commonly producing non-viable images. Such information may help the user to adjust their behavior in the future to minimize or compensate for such incidents (as well as to help technicians to identify new labels and edge cases).

In the depicted example, various non-viable image classes recognized by the one or more classifiers are presented the list 2405s. Here, the user has selected the occlusion non-viable image class 2405t and the fluid blur non-viable image class 2405u, indicated by the highlighted borders. Respective plots 2405v and 2405w may then be produced in the plotted region 2405z, indicating the occurrence of each selected non-viable image classification type over the course of the procedure for a population of surgical operators. Thus, the timeline 2405x may correspond to the timeline 2405m and similar adjustment of the indicator 2405y may adjust the playback accordingly. A bar chart, or other suitable representation may also be used. Similarly, one will appreciate that rather than the raw number of non-viable image counts for the class, the cumulative count, average counts within a sliding window, and other representations of the data may be used to generate the plots in region 2405z. In some embodiments, the average result from a corpus of the similar surgeries, by the same operator or other operators may likewise be overlaid, to provide relative context.

Playback may be integrated across the GUI elements. For example, FIG. 24B provides a flow diagram illustrating various operations in a process 2410 for responding to a user playback position selection (e.g., clicking and dragging the indicator 2405n), as may be implemented in some embodiments. At block 2410a, the system may receive the newly selected playback position. At block 2405b the corresponding portions of the model(s) (e.g., model 2405b or 2405e) may be highlighted. For example, the representation of the camera's position 2405m may be adjusted to indicate the location and orientation of the camera at the selected playback position. Similarly, the appropriate region may be indicated in the representation 2405b. Where a task corresponds to the current playback position, the corresponding icon may be highlighted in the list 2405a. At block 2410c, video playback in the region 2405k may likewise be adjust to the newly selected position. At blocks 2410d and 2410e, the system may retrieve records within a threshold window of the newly selected playback time and may present them to the user. For example, the classification for the presently depicted frame may be updated in the indication 24051, corresponding popups on the model, such as the popup 2405h, may be presented or highlighted, peer data may be presented in the plots of region 2405z, etc.

Similarly, just as the user may isolate data of interest by selecting a temporal location, FIG. 24C is a flow diagram illustrating various operations in a process 2415 for choosing information via a spatial selection, as may be implemented in some embodiments. Specifically, at block 2415a, the system may receive a spatial location, such as by clicking, e.g., using cursor 2405j, upon the model 2405e or a region of model 2405b, etc. As multiple points in time may correspond to the same location (e.g., a colonoscope may often pass through a region at least twice, once for insertion and once for withdrawal), at block 2415b, the system may highlight portions of the timeline (e.g., the timeline 2405m) where the camera passed within a threshold distance of the selected location. In some embodiments, playback may be adjusted to the first of such temporal locations at block 2415c (or the playback may be adjusted in response to the user's selection of one of the highlighted timeline regions), though, again, it may be possible to select advancing and withdrawing encounters into a region only, based upon the direction of motion and point of entry into the region. Similar to the temporal selection of records in process 2410, at blocks 2415d and 2415e the system may retrieve records associated with the selected location. For example, the classification for the presently depicted frame may again be updated in the indication 24051, corresponding popups on the model, such as the popup 2405h may be presented for highlighted, peer data may be presented in the plots of region 2405z, etc.

### Iterative Internal Body Structure Representation—Overview

Various of the embodiments disclosed herein contemplate a surgical navigation service facilitating real-time navigation during a surgical procedure, e.g., as in surgical theater 100a or surgical theater 100b (though one will appreciate that some embodiments may readily be applied mutatis mutandis during post-surgery review). Such a system may monitor progress throughout the surgical procedure and provide guidance to a control system or human operator in response to the state of that progress. For example, in a colonoscopy, the navigation system may direct the operator to un-inspected regions of a patient interior, such as a colon, and may determine coverage estimates for the procedure, such as the remaining percentage of the colon believed to remain uninspected. Coverage, as described in greater detail herein, may be estimated, e.g., by comparing two extreme points in which a colonoscope camera has traveled with an estimated overall length of the colon under examination. Various graphical feedback methods are likewise disclosed, herein, with which the system may advise the operator or reviewer of the procedure's state of progress. While many of the examples disclosed herein are with respect to the colonoscopy context, one will readily appreciate applications mutatis mutandis in other surgical contexts (e.g., in pulmonary contexts such as the examination of bronchial pathways, esophageal examinations, arterial contexts during stent delivery, etc.).

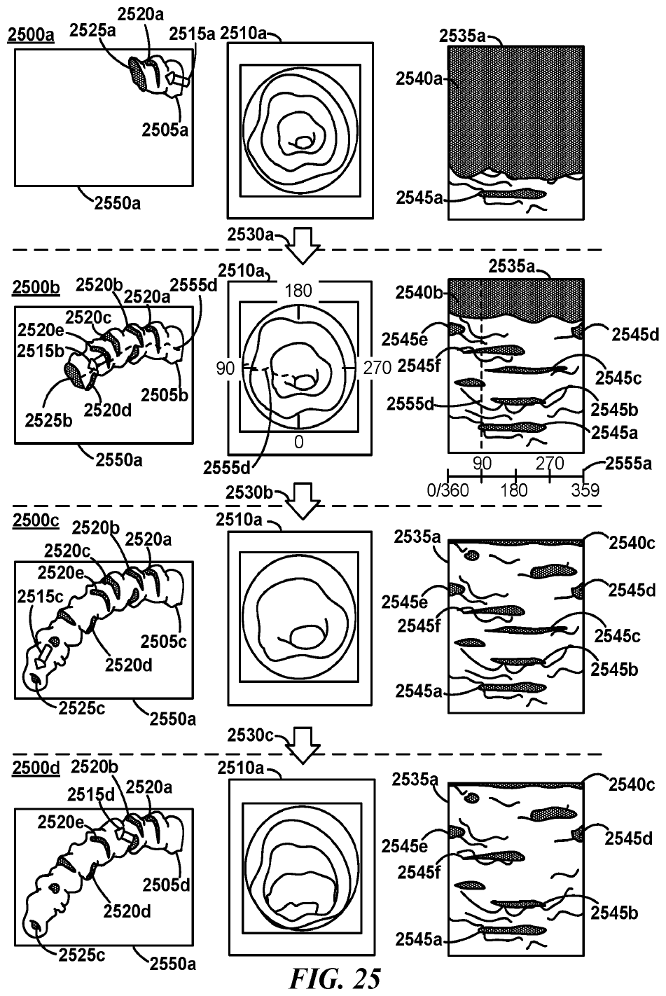

FIG. 25 is a schematic sequence of states for model, view, and projected mapping regions of a GUI in a coverage assessment process, as may be implemented in some embodiments. Specifically, a graphical interface presented to one or more of surgeons 105a or 105c, or assisting members 105b, 105d, e.g., on display 125, display 150, display 160a, etc., or, e.g. to a reviewer examining surgical data post-surgery upon a desktop, may include one or more of the model, view, and projected mapping regions in windows, frames, translucent overlays, or other display areas, such as a headset. In some embodiments, the views may be displayed simultaneously or may be individually and alternately displayed with a selector (thus, e.g., different of the displays 125, 150, 160a, etc. may display different of the views simultaneously during a surgery).

At a first time 2500a, the system may present to the user one or more of: a model region 2550a depicting a partially constructed three-dimensional model 2505a of an internal body region (here a portion of an intestine); a view region 2510a depicting the camera view of a surgical instrument, and projected mapping region 2535a depicting a two-dimensional “flattened” image of the internal body region's surface (here, the interior texture of the intestine; uninspected regions where no surface texture has yet been assigned may be indicated with, e.g., black pixels). Projected mapping region 2535a may be used to infer the state of coverage (e.g., with a percentage of the entire region covered in lacuna). For clarity, each of the GUI regions 2550a, 2510a, 2535a, may appear upon one or more of display 125, display 160a, display 150, a separate computer monitor display, etc.

While region 2510a may depict the output from a surgical camera, such as a colonoscope, each of regions 2550a and 2535a may depict corresponding representations with portions reflecting inadequately examined regions of the patient interior. In some embodiments, inadequate examination may comprise regions which have not yet been directly viewed using the surgical camera (e.g., as they were occluded by an intestinal fold or surgical instrument). In some embodiments, though, the inadequate regions may be regions insufficiently viewed for the given surgical context (e.g., a polyp search may require a minimum time for viewing a given region, tissue recognition with a neural network may require minimal blur, etc.), viewed without proper filtering, for an improper duration, improper laparoscopic inflation, improperly dyed, etc.

In the depicted example, a portion 2520a of the incomplete model 2505a, has not yet been adequately viewed with the surgical camera by the operator. The portion 2520a may be identified in the region 2550a via an absence of model faces, faces with a specific texture or color highlighting the lacunae, an outlining of edges corresponding to the omitted faces of the model, etc. Similarly, for an incomplete model, a large portion 2525a beyond the camera or depth determination system's range may appear in the model 2505a at time 2500a. Each of the lacunae 2525a and 2520a may have corresponding representations in the flattened image of region 2535a, specifically the regions 2540a and 2545a of the flattened image, respectively. That is, as the model 2505a is progressively generated while the surgical camera passes through the patient interior, the corresponding texture map of the interior may be “unrolled” onto the two-dimensional surface of region 2535a (analogous to a UV mapping of texture coordinates between faces of a three-dimensional model and a two-dimensional plane). In some embodiments, a navigation arrow or other icon 2515a may be used to notify the reviewer of the current, relative orientation of the camera providing the view in region 2510a from the perspective of the model 2505a (as shown in this example, occluding faces of the model may not be rendered around the icon 2515a, though one will readily appreciate variations, e.g., where the icon 2515a is rendered upon a billboard between the model and the reviewer, intervening model faces are rendered translucently, etc.). As indicated, the portion of the region 2535a outside the lacunae 2545a and 2540a may be rendered with the intestine texture acquired using the camera. For clarity, though localization and mapping are shown here occurring during the colonoscope's forward advance through the colon, resulting in the creation of additional model segments, one will appreciate that in some colonoscope operations, mapping and localization may be performed only during withdrawal, or the mapping in withdrawal may supplement the results from the advance.

As the region 2550a depicts the model 2505a from a three dimensional perspective, it may be difficult for the operator or assistant to recognize the relative position of the lacunae from the region 2550a alone. While translucent faces, billboards, and other graphical approaches (e.g., such as that described to render the fiducial 2515a) upon the model 2505a may be readily used to highlight lacunae to the operator on opposite sides of the model, or in locations occluded by the present perspective view of the model, such approaches may become confusing in the presence of multiple lacuna. Similarly, inviting the operator or an assistant to rotate or translate their perspective relative to the model 2505a to confirm the relative location of the lacuna under the time constraints and other priorities of the surgical procedure is often not ideal. Thus, the two-dimensional representation of 2535a facilitates a quick and more intuitive guide by which the operator or reviewer may readily assess the present situation.

Accordingly, as time progresses 2530a to a subsequent time 2500b, each region may change its state in accordance with the progress of the surgical procedure. Here, at time 2500b, region 2550a now depicts a supplemented partial model 2505b, the region 2510a depicts the camera's field of view in a more advanced position in the intestine, and the region 2535a depicts more textured surfaces. As the operator has advanced the camera without taking time to remedy the lacunae as they are encountered, new lacunae appear in the model, including new lacunae 2520b, 2520c, and 2520d. Relatedly, the lacuna 2525b corresponding to the yet unexamined region has replaced lacuna 2525a and the arrow icon has advanced to the new orientation 2515b corresponding to the advanced position the camera.

The updated representation in region 2535a will reflect the existence of the newly introduced lacunae. For example, the lacuna 2520b corresponds to the flattened region 2545b, the lacuna 2520c corresponds to flattened region 2545c, and the lacuna 2520d corresponds to the flattened regions 2545d and 2545e. One will appreciate that in the depicted example, coordinates from the approximately cylindrical structure of the model are being mapped to the region 2535a (appreciating that the depiction here is schematic). Thus, while the vertical dimension of the region 2535a corresponds to the colonoscope's longitudinal progress, the horizontal dimension of the region 2535a maps to the 360 degrees of the approximately cylindrical intestine.

Thus, for the reader's convenience and so as to further facilitate understanding (though as will be discussed, similar overlaid indicia may be provided to the operator in some embodiments), at time 2500b a reference 2555a is shown in the figure, relating the 360 degrees of the camera's field of view upon the horizontal access of region 2535a with the 360 degrees in the camera's field of view in region 2510a. As shown, the top of the camera is taken in this example as being at the 180 degree location, corresponding to the center of region 2535a's horizontal dimension. Conversely, the 0 and 360 positions (being equivalent) in the camera view of region 2510a correspond to the left-most edge of the region 2535a. Because the mapping results in the “bottom-most” position in the colon field of view appear as a wraparound at the edges of region 2535a, lacunae such as lacuna 2520d appearing at the “bottom” location of the camera may correspond to two regions, specifically regions 2545d and 2545e along the same horizontal row or rows of region 2535a, but upon opposite edges of region (i.e., they refer to the same lacuna 2520d).

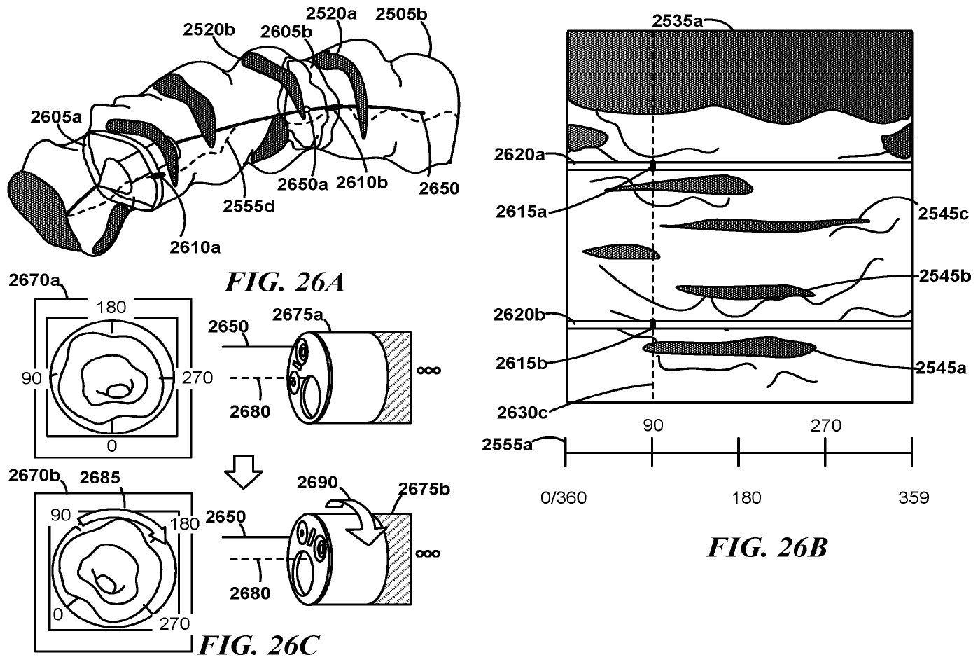

For further clarity, FIGS. 26A and 26B depict enlarged views of the model and projected mapping states, respectively, at time 2500b in FIG. 25. Here, two circumferences 2605a and 2605b of the model 2505b corresponding to rows 2620a and 2620b in the region 2535a are shown (again, one will appreciate that the views are schematic and the mapping between, e.g., lacunae 2520a and 2520b to regions 2545a and 2545a, are not exact). Each circumference 2605a and 2605b may be determined, e.g., as comprising the closest points upon the model 2505b in a circle about a point upon the model's centerline 2650 (e.g., the medial axis centerline, as determined manually, as determined programmatically based upon model moments, as inferred from colonoscope kinematics, etc.). Here, for example, the circumference 2605b is determined by the point 2650a on the centerline 2650. Each row in the image of region 2535a may thus be determined by a corresponding circumference upon the model 2505b. Accordingly, along the 90 degree line of continuity 2555d (corresponding to the reference line 2630c) the portion 2610a of the circumference 2605a corresponds to the point 2615a on the row 2620a. Similarly, the portion 2610b of the circumference 2605b corresponds to the point 2615b on the row 2620b. One will appreciate that portions of the circumference encountering a lacuna will likewise precipitate a lacuna region in the corresponding portion of the row in the projected map region 2535a. For example, having determined a point upon the centerline and seeking the closest vertex to a ray extending from the point into a given radial direction (e.g., 90 degrees), if no model vertex is within a suitable threshold distance of the ray, then the radial direction may be associated with the non-texture or lacuna value (e.g., the pixel in the column of the row corresponding to the circumference associated with the radial direction may indicate a lacuna rather than a captured texture value.)

One will appreciate that because the mapping process may be substantially temporally continuous, the system may be able to infer the camera's orientation relative to previously mapped sections and relative to its current field of view. Similarly, because circumferences are determined from the model's vertices and centerline, rather than from the camera's current position, the camera may assume a variety of orientations without disrupting the three-dimensional model or corresponding projected map generation. That is, the system may readily assign degrees to model vertices in the circumference even if the camera does not enter a region at any particular angle and even if only part of the circumference is visible. In this manner. the presence of circumferences may facilitate a “universal” set of coordinates for the operator.

For clarity, FIG. 26C is a schematic representation of a pair of relatively rotated surgical camera orientations 2675a, 2675b and their corresponding fields of view 2670a, 2670b. Initially, the surgical camera may be in a first orientation 2675a slightly below and to the left of the three-dimensional model's centerline 2650. Naturally, this may produce the field of view 2670a, similar to what has been discussed with respect to FIG. 25. When the camera is rotated 2690 counter-clockwise about its longitudinal axis 2680, naturally, the field of view 2670b will correspondingly rotate. However, as indicated by the countervailing arrow 2685, the system will continue to construe images acquired from the camera relative to the original orientation. Thus, whether the camera advances in the orientation 2675a or in the orientation 2675b, the system will produce the same image in region 2535a, as the same circumferences in the model will be generated with the same corresponding rows and columns in the image.

Returning to FIG. 25, as time again advances 2530b to the time 2500c and the surgical procedure continues, region 2510a may depict the camera's more advanced field of view, region 2550a may show a correspondingly more complete model 2505c, with the icon corresponding to the present camera orientation at an advanced position 2515c, and the region 2535a may show a mostly fully textured two-dimensional plane (e.g., where the full length of the model corresponds to full length of the expected portion of the colon to be examined). In this example, as the end of the intestine remains open, a lacuna 2525c remains corresponding to a residual lacuna region 2540c. In this example, rather than correct lacunae along the way, the operator has elected to return to an earlier portion of the examination after an initial progression and to then remove a lacunae at that location by inspection.

Specifically, as time advances 2530c to the time 2500d, the user has elected to resolve lacuna 2520c (corresponding to the region 2545c) and therefore returned to the location of lacuna 2520c and brought the missing portion of the intestine within the camera's field of view as shown in the region 2510a. By bringing this region into view, the corresponding lacuna is removed from the model in the view 2550a (one will note that the fiducial's orientation 2515d is pointing to where lacunae 2520c had previously been at time 2500c). This precipitates a new model version 2505d wherein the lacuna has been filled. Similarly, the region 2545c corresponding to the lacuna 2520c is likewise omitted from the view 2535a at time 2500d.

### Iterative Internal Body Structure Representation—Example Processing Overview

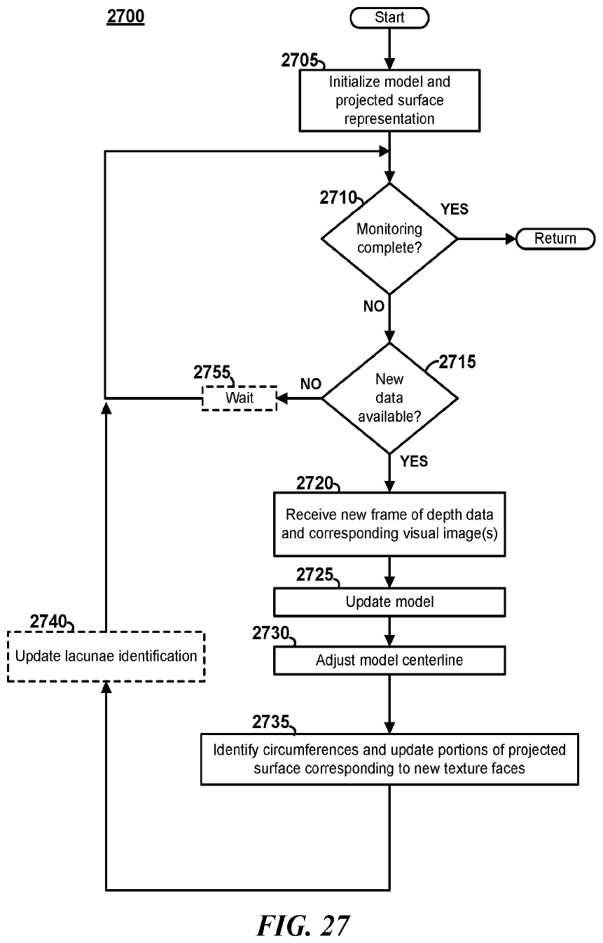

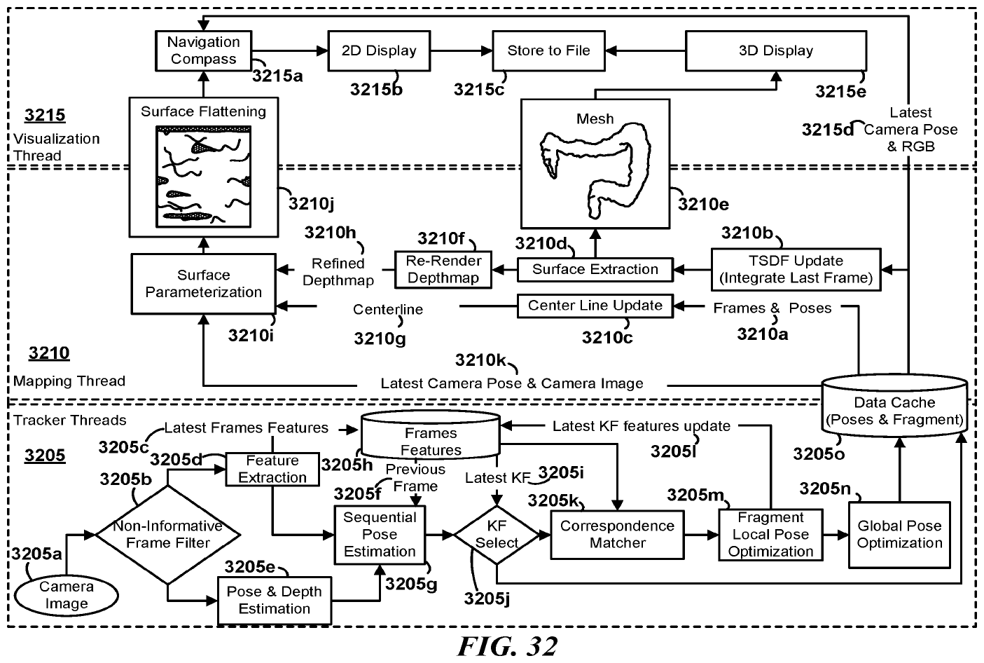

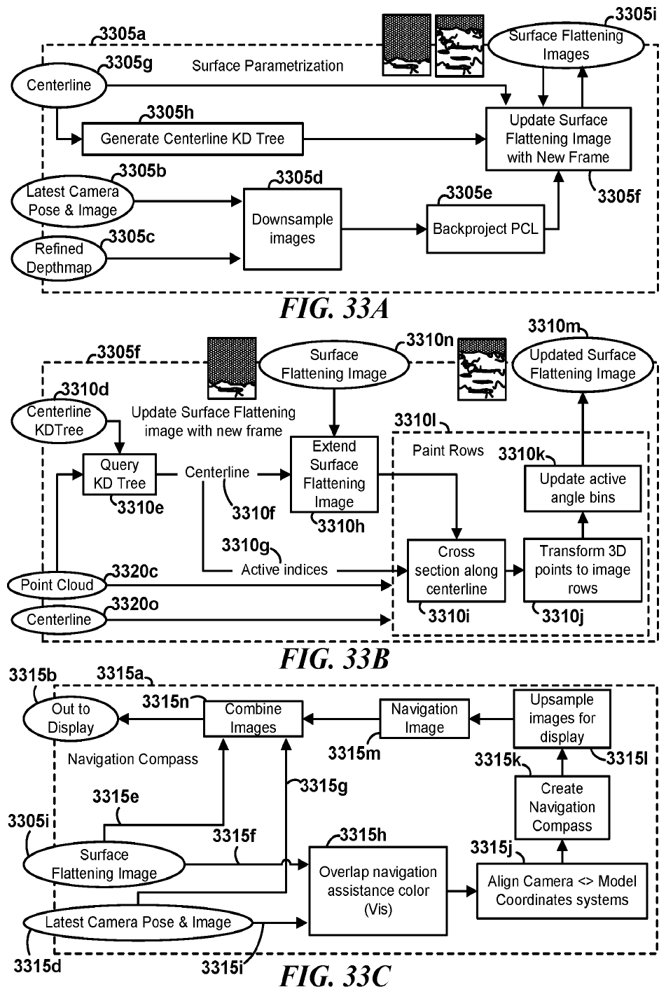

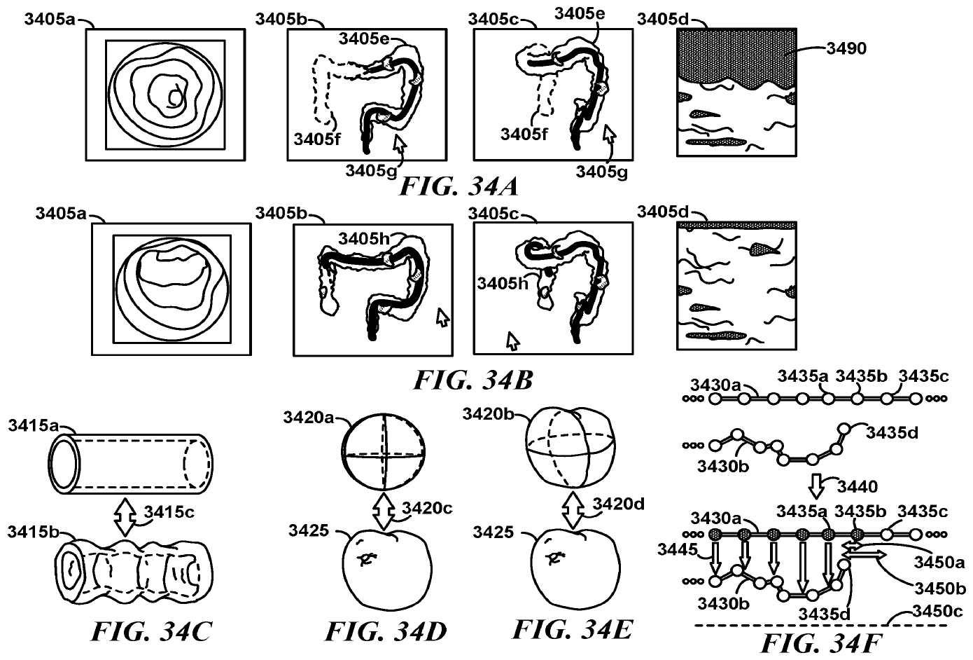

FIG. 27 is a flow diagram illustrating various operations in an example process 2700 for performing the coverage assessment process of FIG. 25. While various operations may be discussed herein in greater detail with respect to FIGS. 32 and FIGS. 33A-C, FIG. 27 provides a general overview. Specifically, during the surgery, the system may be initialized at block 2705, preparing an initial model mesh (e.g., an empty space, or a space with a guide structure, as discussed herein with respect to FIGS. 34C-F) and surface projection (e.g., where the entire image is a single, monolithic lacuna). Though a two-dimensional rectangle was depicted and discussed with respect to FIG. 25 as the projected image region, as discussed elsewhere herein, one will appreciate that the mapping may be to surfaces other than a two dimensional rectangle.

At block 2710, the system may determine whether monitoring of the surgical process is complete. For example, the operator may not desire lacunae recognition at all times throughout the procedure or the procedure may conclude. If the monitoring has not yet concluded, then at block 2715, the system may determine whether new depth frame and image data are available, and if such data is not yet available, wait as indicated by block 2755.

Once new data is available, the system may acquire the new image and depth data at block 2720. At block 2725, the system may then update the model with the depth data, e.g., extending the model 2505b to the new partial model 2505c. With the updated model, it may be possible to extend the centerline at block 2730 using the new model vertices (again, one will appreciate alternative methods for extending a centerline, e.g., based upon camera motion, encoders, etc.). The extended centerline may in turn be used at block 2735 to determine new circumferences (e.g., one of circumferences 2605a or 2605b). The vertices of the circumferences are themselves associated with faces, which may themselves be associated with texture coordinates from the visual images. Thus, the system has a ready collection of references by which to infer the row pixel values (e.g., the pixel values in rows 2620a and 2620b). For example, each of the 360 degrees in the circumference may be used to identify the pixel value for the corresponding column in the circumference's row (or, as mentioned, infer a lacunae value for a given radial direction).

In some embodiments, as the lacunae may be self-evident to the user from the rendering, the system may not recognize lacunae explicitly. However in some embodiments, at block 2740, the system may recognize lacunae, either for use in an internal process and/or for highlighting to the operator. One will appreciate that lacunae recognition may be performed in a number of ways upon either the model or upon the projected image. For example, flood fill algorithms and blob analysis provide ready methods for determining groups of pixels associated with lacunae in the projected image. Just as circumferences in the model were used to infer texture values for populating rows in the projected image, once lacunae are recognized in the image, the system can look back to the corresponding circumference for that row to recognize the three-dimensional location of the lacuna. As another example, regions of the model lacking a threshold number of nearby vertices may likewise be construed as a lacuna.

### Graphical Supplements for Navigation and Orientation

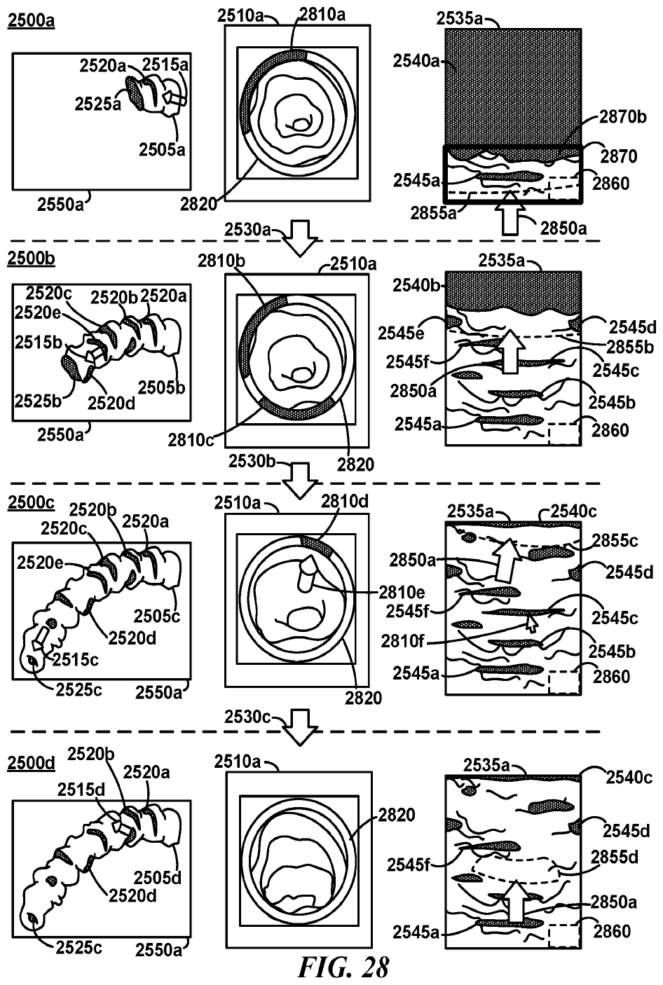

FIG. 28 is a schematic sequence of states for model, view, and projected mapping regions of FIG. 25, but with additional graphical guides, as may be implemented in some embodiments. For example, view region 2510a may include a directional compass 2820 notifying the operator of nearby lacuna (as well, in some embodiments, as indicating the camera's current relative rotation to the nearest circumference, e.g., via a hue-to-radial direction correspondence). Similarly, the projected mapping region 2535a may include a local indicator 2850a showing the current relative position and orientation upon the projected image of the camera depicting the present view in view region 2510a. The location of the camera may be visualized by the local indicator 2850a, e.g., as a circle upon the map 2535a in a color different from the body interior texture, or other indicia, such as an arrow, shown here in a first position. In some embodiments, the portion of the image presently appearing in the camera view may also be indicated with indicia 2855a, e.g., with an outline, change in luminosity of the image pixels, colored border, etc. A bottom-right portion 2860 of the region may include a textual overlay indicating various monitoring statistics. For example, the textual overlay may indicate a percentage of the intestine which is unmapped, or which is already mapped, relative to a standard reference, a ratio of lacuna to completed portions of the model, a length of the intestine in centimeters which the camera has so far traversed, a number of existing lacunae, etc. (e.g., an insertion depth “13 cm” as the current length of the global centerline and a coverage score “67.1%” per the ratios described herein). A length traveled, and length corresponding to the unmapped or mapped percentage of the intestine may also be displayed. Length may be determined for the global centerline at each point in time, as, e.g., the geodesic distance between the 2 extremities of the global centerline.

A depicted coverage score may be determined as a ratio of the mapped colon over the total predicted mapping area (e.g., averaged from a corpus of colon models and scaled by the patient's dimensions). Local coverage, such as of only the current segment of the colon in which the colonoscope is present, or of only a magnified region, such as magnified region 2970b discussed infra, may also be depicted. As yet another example, a coverage score may be calculated for only the previously surveyed region, so as to notify the surgical team of the surface area's lacunae. That is, with reference to time 2500a, a rectangle 2870 (also referred to as the “presently surveyed area”) on the image region 2535a indicates the portion of the image region 2535a used for the coverage calculation, where the width of the rectangle 2870 is the same as the width of image region 2535a and the height of the rectangle 2870 corresponds to the furthest row from the starting row containing a mapped pixel value. The coverage score in this rectangle may be determined based upon a ratio of the lacune and non-lacuna pixels in the rectangle 2870, e.g., the number of pixels associated with lacuna (including unmapped portions in the rectangle 2870 where, as here, the terminating surface of the mapped region is not flush with rectangle 2870) in the numerator and the total number of pixels in the rectangle in the denominator (or, conversely, the total pixels in the rectangle minus the lacunae associated pixels in the numerator and the total pixels in the rectangle 2870 in the denominator). Thus, the score in the depicted example is the ratio of the sum of the pixels in lacuna region 2545a and the pixels in the unmapped region 2870b within the rectangle 2870, divided by the total number of pixels in the rectangle 2870.

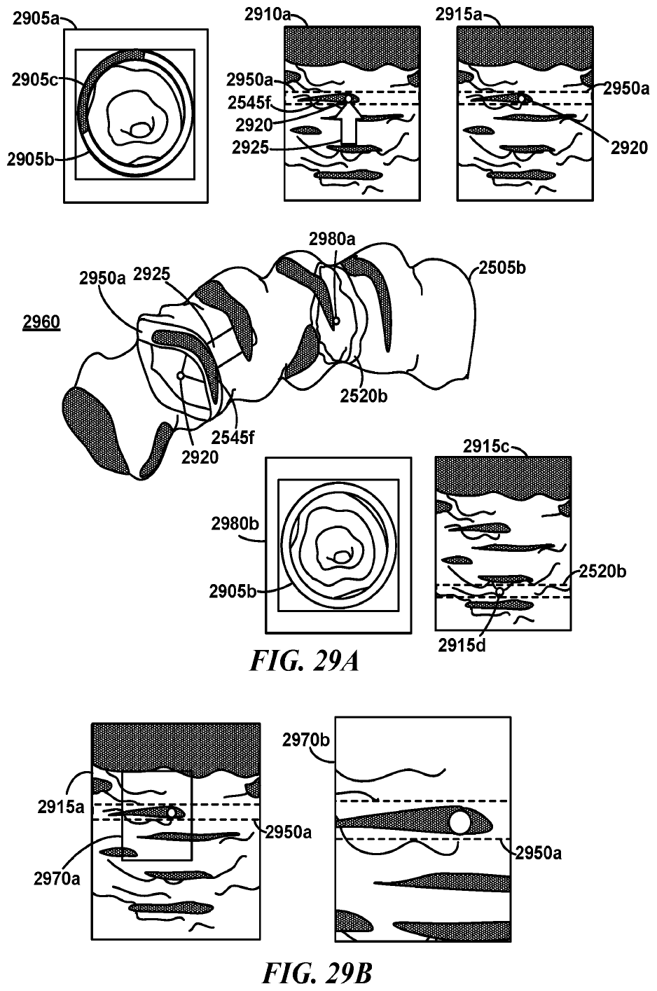

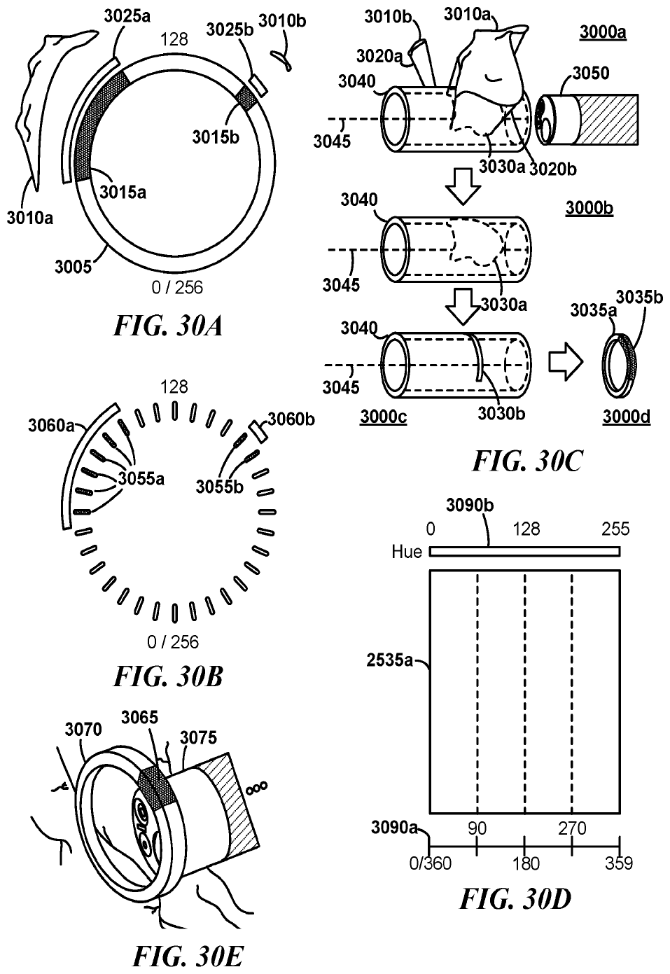

The local indicator 2850a or compass 2820 may direct the user to look in the direction of a lacuna so as to remedy a deficiency. Here, e.g., at time 2500a a portion 2810a of the compass 2820 is highlighted to inform the user that the lacuna 2520a is above and slightly to the left of the current field of view (in some embodiments, the lacuna region 2545a may likewise be highlighted). As will be discussed in greater detail herein, the system may consider one or more of the following in determining which direction to recommend in compass 2820: the input camera image; the predicted depth map; the estimated pose of the camera in determining its recommendation; and a centerline from a start of the sequence (e.g., in the cecum, if the operation is being performed during withdrawal) to the current camera position. For example, the system may consider points along the centerline and corresponding circumferences within a threshold distance of the camera's current position. Lacuna falling upon those circumferences may then produce corresponding highlights (e.g., highlight 2810a). In some embodiments, all of these lacuna precipitate the same colored highlight 2810a in the compass 2820. However, in some embodiments, lacuna in front of the camera may be highlighted in a first color (e.g., green) and lacuna behind the camera may be highlighted in a second color (e.g., red), to provide further directional context. As will be discussed with respect to FIG. 30C, color may instead indicate the radial position, and the forward or backward relative position may instead be indicated with the color or pattern of a border surrounding the highlighted portion (e.g., a lacuna behind and at 180 degrees may precipitate a light blue highlight and a red border). As the camera rotates, e.g., as discussed with respect to FIG. 26C, the compass 2820 may rotate within the camera field of view to inform their user of the camera's orientation relative to the model's coordinates. In yet further embodiments, discussed, e.g., in FIG. 29A, the compass 2820 only indicates lacunae in the circumference (or circumferences in a threshold distance) from the camera's present position.

At time 2500b, the user has again advanced the camera further into the intestine, such that the compass 2820 and local indicator 2850a are likewise updated (as well, in this embodiment, as the field of view indicia 2855b). Here, highlight 2810b corresponds to the lacuna 2520e (and region 2545f) and the highlight 2810c corresponds to lacuna 2520d (and regions 2545d and 2545e; for clarity, the portion of lacuna 2520d wrapping under and around the model from the reader's perspective is not shown), as each of these lacuna fall within a threshold distance of the camera's present position. Similarly, in some embodiments these lacuna may be highlighted in the corresponding regions of the image region 2535a.

When, at time 2500c, the user has advanced further into the colon, the local indicator 2850a and indicia 2855c may again be updated. In the depicted embodiments, the user has moved a cursor 2810f over a region 2545c corresponding to a lacuna of interest 2520b. Upon clicking and selecting the lacuna, the system may ignore the local threshold criteria for updating the compass 2820 and instead provide highlights so as to direct the user to the selected lacuna, here, the highlight 2810d. In some embodiments, overlays, augmented reality projections, etc. may also be integrated into the region 2510a. For example, here, a three-dimensional arrow 2810e has been projected into the space of the field of view to direct the user toward the selected lacuna 2520b.

Once the selected lacuna is remedied at time 2500d, the compass 2820 may be cleared of highlights, as in the depicted embodiment. In some embodiments, the system may instead revert to depicting other lacunae in the vicinity within compass 2820. For clarity, because the user is looking at the “ceiling” of the intestine, the indicia 2855d encompasses only the corresponding central portion of the two-dimensional image.

### Graphical Supplements for Navigation and Orientation—Example Local References

As discussed herein, in some embodiments, the system may recognize lacunae appearing ahead and behind the colonoscope's position and call the surgical team's attention to the same. In some embodiments, however, lacunae identification may be localized to particular circumferences (e.g., circumferences 2605a and 2605b) in the model. That is, in contrast to the methodology discussed herein with respect to FIG. 30C infra, wherein highlights and other regions are identified ahead or behind the colonoscope's current position, some embodiments may instead limit notification to specific circumferences, such as the circumference in which the colonoscope is presently located.

For example, FIG. 29A depicts a pair of schematic and projected mapping regions for a local compass scope, as may be implemented in some embodiments. Here, the system overlays a compass guide element 2905b upon a GUI element 2905a depicting the colonoscope's present field of view. Here, the compass 2905b is directing the operator's attention to the upper left quadrant of the colonoscope's field of view via highlight 2905c (e.g., corresponding to the presence of the lacuna 2545f). This is also reflected in the two-dimensional projected map 2910a wherein only lacuna intersective, at least in part, circumferences, e.g., circumference 2950a, within a threshold distance of the colonoscope's current position are considered for representation with a corresponding highlight in the compass 2905b.

In this example, the lacuna 2545f falls entirely in the one or more considered circumferences (here, circumference 2950a). Where only a portion of a lacuna falls within the circumference, only that portion of the compass corresponding to the intersecting lacuna portion may be highlighted. For clarity, as shown in perspective view 2960, the circumference 2950a is shown relative to the incomplete three-dimensional model 2505b. Thus, the compass 2905b may not display highlights, such as highlight 2905c, until the colonoscope is placed in such as position (e.g., the position associated with the point 2920). Such embodiments may be useful, e.g., in surgical procedures where inspection occurs during withdrawal. That is, in some surgical procedures, the initial advance to a terminal point near the cecum is mostly performed only to prepare for a subsequent procedure, such as inspection of the colon. Localization, mapping, and lacuna remediation then occur in tandem with the slow withdrawal and inspection. Circumference by circumference verification may facilitate a more methodical review than relying solely upon the operator's judgment. Thus, in addition to lacunae resulting from an incomplete model, regions of the model and projected map corresponding to regions that the colonoscope has not viewed for an adequate amount of time, may likewise be called to the operator's attention in the same manner as model lacunae.

As shown in two-dimensional projected map 2910a, an arrow indicia 2925 or a single dot 2920 may be used to indicate the present position of the camera and its relation to the circumference 2950a. Use of a single dot, or other small marker, exclusively, as shown in two-dimensional projected map 2915a, may be used to provide less obtrusive representations of position. Similarly, in some embodiments, neither a dot nor an arrow are presented (i.e., no point-based indicia), but only the rows of the two-dimensional projected map corresponding to circumferences within the threshold distance of the current colonoscope position are noted (e.g., via a rectangular bounding box, via a change in luminosity of the rows relative to other rows, etc.). Naturally, highlighting of the circumferences may be combined with a position or orientation as well.

For completeness in the reader's comprehension, also shown in this example is the circumference 2520b. If the colonoscope were in the position 2980a the element 2905a may appear as shown in the state 2980b, and the compass 2905b may not include any highlights. Similarly, the two-dimensional projected map 2915c will indicate that for the current camera position, indicated by the dot 2915d, the nearest circumference 2520b or circumferences within a threshold distance of the current position do not intersect a lacuna.

Using the approaches disclose herein, one will appreciate that operators may sometimes benefit from GUI elements presenting portions of the model and projected map in varying levels of detail. For example, FIG. 29B is a projected mapping GUI element with a level-of-detail magnification, as may be implemented in some embodiments. Specifically, the example projected map element 2915a of FIG. 29A is shown here with a bounding box 2970a indicating a portion of the element 2915a appearing in a magnified region 2970b. One or more increasing levels of detail, as in magnified region 2970b, may be presented to the surgical team, e.g., overlaid upon map element 2915a, concatenated to mapping map element 2915a, shown overlaid a different GUI element, etc.

Magnified region 2970b may help the operator to align the camera position relative to a circumference, e.g., circumference 2950a. This may facilitate the local remediation of lacunae at a higher resolution than that used for the more global representation of map element 2915a. As described with respect to the embodiments of FIG. 29A, the navigation compass may call attention to missing regions “encircling” the camera. Indeed, in some embodiments, portions of the rectangle 2950a corresponding to the one or more circumferences may have regions highlighted in correspondence with the highlights of the compass (e.g., where the top, 180 degree, position of the compass is highlighted, then the center of the rectangle 2950a may be likewise highlighted in the magnified region, though the rendering of the lacuna may already make the correspondence clear). Thus, the operator may compare the feedback from the compass with the representation of the circumference, e.g., circumference 2950a in the magnified region 2970b.

In some embodiments, the magnified region may automatically follow the region around the colonoscope's current position. Such behavior may be useful in situations where extending and projecting an image at only one resolution may result in degradation of various details, making it difficult, e.g., for the surgical team to recognize small lacunae.

### Example Variations in Graphical Supplements

For further clarification, FIG. 30A is a schematic representation of a continuous navigation compass 3005 (here represented in circular form) as may be implemented in some embodiment. Here, an example lacuna 3010a is ahead and in the upper left quadrant of the camera's field of view, with corresponding highlighted region 3015a upon compass 3005. Similarly, lacuna 3010b is ahead and in the upper right quadrant of the camera's field of view, with corresponding highlighted region 3015b upon compass 3005. In the depicted embodiment, the size of the highlighted regions 3015a and 3015b correspond to the projected size of the corresponding lacuna.

While there are many ways for the highlights and the lacunae to correspond, FIG. 30C provides a schematic representation of a series of states in determining a relative position for displaying a highlight, as may occur in some embodiments. Specifically, as shown in state 3000a by envisioning a cylinder 3040 around the centerline 3045 of a colonoscope 3050's field of view, the system may infer the corresponding portion of the compass to highlight for a given lacuna (though only a portion of the cylinder in front of the camera is shown, one may readily appreciate variations wherein the cylinder extends behind the camera to accommodate projections of lacuna behind the camera). For example, cylinder 3040 may have the same radius as the navigation compass (e.g., compass 3005). The system may project 3020a lacuna 3010b and may project 3020b lacuna 3010a onto the surface of the cylinder 3040. This may precipitate a projected shape 3030a upon the cylinder surface, as shown in state 3000b. By condensing this shape along the circumference of the cylinder, or by considering the radial boundaries in the cylinder, a limiting shape 3030b may be inferred from the shape 3030a as shown in state 3000c. The limiting shape 3030b or corresponding boundary may then be mapped to the dimensions of the compass 3035a to determine the highlighted portion 3035b as shown in state 3000d. One will appreciate that this is but one example, and highlights may readily be determined by other projections (e.g., a direct projection upon a circle in the plane of the camera's field of view).

As mentioned, in some embodiments, as was discussed in connection FIGS. 29A-B where only lacuna intersecting the radial direction of the circumference in which the colonoscope camera presently resides may be depicted on the compass, there may be no need to distinguish between lacunae along the longitudinal axis. In contrast, in embodiments corresponding to the example of FIG. 30C where lacunae ahead or behind of the colonoscope's present position may be represented on the compass, overlapping lacuna may be distinguished by differing colors, boundary outlines, indications of the number of lacunae in the region, etc. In some embodiments, each lacuna, or at least each lacuna under consideration, may be assigned a distinct color, or other unique identifier, and these unique identifiers then used to distinguish lacunae representations within the compass.

Returning to FIG. 30A, the highlights 3015a and 3015b may thus be determined in the manner described with respect to FIG. 30C. As mentioned, the highlights may not be the same color and may indeed depict a variety of colors to reinforce the relative positions of the camera and lacuna. For example, as shown in FIG. 30D, the 360 range of radial directions in the model may be assigned a color (one will appreciate that the granularity may be varied and, consequently, that the mapping from hue to angle may be continuous or discretized at varying levels). Where colors are represented with Hue, Saturation, and Luminosity (HSL), one will appreciate hat hue, when represented by eight bits, may thus assume a value between 0 and 255 (accordingly, red may correspond to a value of 0, green to 90, blue to 170, etc. FIG. 30D illustrates the correspondence between the radial degrees from 0 to 360 via reference 3090a to the range of 0 to 256 hue values shown by reference 3090b. Thus, portions of the top of the compass may take on light blue and green values, whereas portions at the bottom of the compass may take on reddish values.

One will thus appreciate that even where the camera is rotated, as was discussed with respect to FIG. 26C, compass 3005 will likewise rotate within the field of view in the countervailing direction to maintain the proper orientation within the space of the three-dimensional model and region 2535a. Similarly, as previously discussed, the compass may include additional indications to help the user recognized the relative position of the lacunae. Here in FIG. 30A, additional relative references 3025a and 3025b are shown outside the corresponding highlights 3015a and 3015b. References 3025a and 3025b may be colored or textured to indicate that the highlighted lacuna are ahead or behind of the camera's present field of view (here, as both lacuna are ahead of the camera, the references may share the same forward indication). One will readily appreciate variations, e.g., in lieu of references 3025a and 3025b, the borders of highlights 3015a and 3015b may indicate the lacuna's position, via color, transparency levels, luminosity, animation, etc.

As mentioned, the compass 3005 and its highlights may be translucent in some embodiments to facilitate a proper field of view by the operator. However, the limited lighting of the body interior may make it difficult to discern the state of the compass during the operation. Accordingly some embodiments consider a compass in the form shown in the example of FIG. 30B, where the radial directions are represented by discrete indicators rather than a continuous compass. As with the compass of FIG. 30A, the indicators may be color coded in accordance with the radial position (e.g., the topmost indicator taking on a value of 128 in the 0-255 hue range, and the bottommost indicator taking on a hue value of 0 in the 0-255 range). Here, the indicators 3055a and 3055b are shown as highlighted (corresponding to the highlights 3015a and 3015b). While references 3060a and 3060b may also be provided in the same manner as references 3025a and 3025b, one will again appreciate that in addition to separate longitudinal references, the indicators may instead be bolded with colors or patterns indicating the relative longitudinal position of the lacunae.

One will appreciate that multiple cameras may be used in some surgical procedures. In some embodiments, augmented reality graphics may be introduced into the display of some cameras when their field of view encompasses another camera with a compass. For example, FIG. 30E depicts a perspective view of a first colonoscope camera 3075 from the perspective of a second surgical camera. To readily facilitate cross-referencing between the views, in this image of the second camera an augmented reality overlay 3070 of the compass seen in the display of the camera 3075 may be presented, including corresponding highlights 3065. In this manner, users may readily cross reference the compass as depicted in the field of view to achieve a holistic understanding of the surgical field, including the relative locations of the camera and any selected lacuna.

### Example GUI Update Process

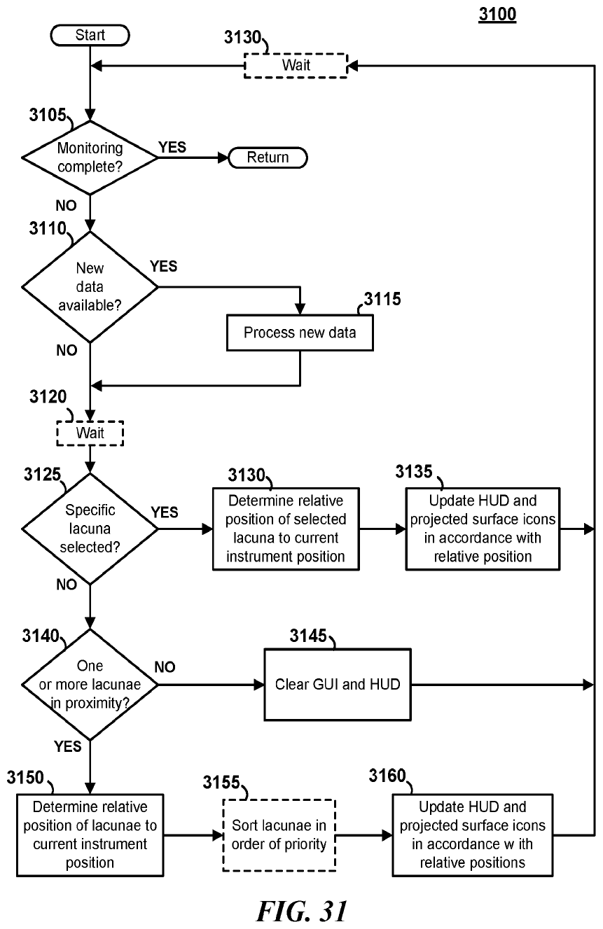

FIG. 31 is a flow diagram illustrating various operations in an example process 3100 for rendering various of the graphical guides of FIG. 28 (process 3100 may run, e.g., as part of the visualization thread 3215 discussed below). At block 3105, the system may determine if monitoring is complete and the process 3100 may conclude if so. Where monitoring is ongoing, at block 3110, the system may determine if new image frames are available (e.g., from the cache 3205o), and if so, process their data at block 3115 (e.g., the operations of block 3215a). As rendering and data processing may occur at different rates, and/or may occur in different threads, the waits of blocks 3120 and 3130 indicate that the process may be delayed on account of the different rates (e.g., in anticipation of the updating of cache 3205o and the preparation of the projected map image 3210j and the updated mesh 3210e).

Thus, prior to rendering, at block 3125 the system may consider if the user has selected any specific lacuna (e.g., as per the cursor 2810f selection of region 2545c). Where a lacuna is selected, at block 3130, the system may determine the relative position of the lacuna to the current instrument position (e.g., using a technique such as that described with respect to FIG. 30C). At block 3135, the system may then update the GUI and projected surface representation to reflect the relative position, such as by indicating highlighted regions of a compass, emphasizing the border of the region in map 2535a, etc.

Where a specific lacuna is not determined to be selected at block 3125, at block 3140, the system may determine if one or more lacunae are in proximity to the camera's current position (e.g., by considering circumferences generated from centerline positions within a threshold distance of the current camera position). If not, then the system may clear the GUI and HUD at block 3145, e.g., to avoid distracting the user. Where one or more lacunae are near, however, at block 3150, the system may determine the relative position of the one or more lacunae to the current instrument position (again, e.g., using the method of FIG. 30C, by projecting mesh positions upon the location of the camera directly, etc.). Where more than one lacunae is in proximity, the system may sort the lacunae by priority at block 3155 (e.g., larger lacunae, or lacunae in sensitive regions of the surgery, may be presented to the user before, or more intensely highlighted, than smaller or less concerning regions). At block 3160, the system may then update the GUI with the appropriate overlays in accordance with the relative position or positions.

### Iterative Internal Body Structure Representation—Example Processing Pipeline

FIG. 32 is a schematic block diagram illustrating various components and their relations in an example processing pipeline for iterative internal structure representation and navigation, as may be implemented in some embodiments. In the depicted embodiments, processing may be generally divided into three portions: visualization, mapping, and tracking. Here, a different computational thread is assigned to each portion, i.e.: a visualization thread 3215, a mapping thread 3210, and a tracker thread 3205. One will appreciate that the threads may programmed to run in parallel on one or more processors, communicating with one another, e.g., using appropriate semaphore flags, queues, etc. Similarly, one will appreciate that each thread may contain sub-threads, as where multiple trackers associated with tracker thread 3205 operate in their own threads.

Beginning with a tracker thread 3205, during the surgical procedure, a new camera image 3205a (e.g., an RGB image, grayscale image, indexed image, etc.) may arrive for processing. At block 3205b, the tracker thread may apply a filter to determine whether the visual image is or is not suitable for downstream processing (e.g., the localization and mapping operations disclosed herein). For example, blurry images, images occluded by biomass or walls of the organ, etc. may be unusable for localization. Where the frame is not usable, it may be discarded (though, in some embodiments, one will appreciate that interpolation or prediction methods between frames may be used to correct some defective frames).

In contrast, where the image is found to be usable at block 3205b, a first copy of the usable image may be provided to pose and depth estimation block 3205e and a second copy of the usable frame provided to feature extraction block 3205d. For example, the features extracted at block 3205d may be scale-invariant feature transform (SIFT) features for the visual image. Again, one will appreciate, e.g., that each of blocks 3205d and 3205e may operate in independent threads, or in sequence in a same thread, in accordance, with, e.g., the methodology described in Posner, Erez, et al. “C3Fusion: Consistent Contrastive Colon Fusion, Towards Deep SLAM in Colonoscopy.” arXiv™ preprint arXiv™:2206.01961 (2022). The extracted features 3205c may be stored in a record 3205h. As discussed elsewhere herein, the images may be used for pose and depth determination at block 3205e. These determinations from block 3205e, the images themselves 3205f as previously stored, and the features extracted at block 3205d may be used to determine the sequential pose estimation of the camera at block 3205g, e.g., as described elsewhere herein. Specifically, the system may compute the sequential pose estimation using the previous frame features 3205f and the latest frame features 3205d. Block 3205g may thus both validate and refine (if needed) the pose estimated by, e.g., one or more convolutional neural networks in block 3205e.

A localization filter, such as a Kalman filter 3205j, may be used to further refine the localization and pose estimate and the result stored in data cache 3205o. As indicated, the Kalman filter 3205j may consider previous Kalman filter 3205i results in its analysis. Even further refinement may be accomplished by providing the matches to a correspondence matcher 3205k and the resulting matching frames sent to local pose optimization block 3205m (which may itself update the record 3205h with the modified Kalman filter results 32051) before performing a global pose optimization at block 3205n (one will appreciate that the modified Kalman filter results 32051 may themselves serve as the latest results 3205i in a subsequent iteration).

With the data cache 3205o populated with the new pose estimation results, mapping thread 3210 may now begin integrating the newly acquired data using the determined pose information. Specifically, frames and poses 3210a may be extracted from the cache 3205o and used for updating the centerline determination at block 3210c (again, one will appreciate a variety of alternative methods for determining the centerline). Similarly, the last depth frame may be acquired for integration with the TSDF structure at block 3210b. From this, the system may then extract the surface of the mesh at block 3210d (e.g., using marching cubes, convex hull, or other suitable approaches) to create the updated mesh 3210e. The mesh surface may also be used for updating the depth map render at block 3210f, so as to produce a refined depth map 3210h. Each of the updated mesh centerline 3210g, refined depth map 3210h, as well as the latest camera pose and image 3210k may then be used to perform surface parametrization at block 3210i to produce the projected surface flattened image 3210j. For example, the system may determine circumferences and corresponding row pixel values corresponding to an active region (e.g., a surrounding region where the colonoscope camera is presently active). Thus, rather than recreate the entire projected image surface with each iterative adjustment to the mesh model, the system may instead update only the active portions of the projected surface flattened image 3210j corresponding to the most recently captured, integrated, and updated portion of the overall 3D model.

As estimated depth maps may sometimes be noisy, some embodiments may create multiple depth maps from the same position, re-rendering the mesh from the same position of the camera in order to create a new, refined depth map. Such local, iterative refinement may be applied, and the operator encouraged by the system (e.g., via GUI feedback) to linger in regions where lacunae appear or where the flattened image or model are poorly structured.

The visualization thread 3215, may then acquire the updated projected image 3210j and mesh 3210e, providing the latter for display at block 3215e and possibly for storage at block 3215c. Similarly, the image 3210j and latest pose and image information 3215d may be used for determining the position and orientation of a navigation compass at block 3215a as described herein (e.g., compass 2820). In some embodiments, it will be at block 3215a that the system ensures that the compass representation retains an appropriate orientation, e.g., to maintain the proper global orientation as was described herein with respect to FIG. 26C, regardless of the camera's roll angle or other change in orientation. The updated compass may then be rendered upon the two-dimensional display 3215b along with the new image 3210j. In some circumstances, the rendered two-dimensional image may likewise be stored to file at block 3215c, e.g., for subsequent review.

### Iterative Internal Body Structure Representation—Example Sub-Processing Pipelines

As discussed above, in some embodiments, navigation itself may build upon the prior pose determination in two stages: surface parametrization, e.g., corresponding to block 3210i, and determination of the navigation compass, e.g., corresponding to block 3215a.

FIG. 33A is a schematic block diagram various operational relations between components of a surface parametrization process 3305a as may be implemented in some embodiments, e.g., at block 3210i. Surface parametrization, as in block 3210i, maps the three-dimensional reconstructed surface of the mesh to a two-dimensional image (such as the projected map of region 2535a). For example, where the mesh is of a colon, surface parametrization may “unwrap” the colon along the centerline of the model, as shown in in FIG. 25, where each horizontal row of the image is derived from mesh vertices along the corresponding circumference derived from the centerline. Similarly, the columns may correspond to angles uniformly sampled by utilizing conformal structure to flatten the colon wall upon a planar image.

For example, as was described with respect to FIGS. 26A-C, the surface parametrization algorithm may determine circumferences by taking cross-sections from the current estimated point cloud at points along the mesh model's centerline. For each vertex appearing in the cross-section, or circumference, the system may assign an angle. Thus, given a centerline 3305g (e.g., centerline 2650) at time t, a K-dimensional tree (KD Tree) may be generated over the centerline point cloud samples at block 3305h. The input depth map 3305c (refined, e.g., per block 3210f) and camera image 3305b may be down-sampled at block 3305d (in a prototype implementation, taking approximately 1 ms to complete). The result may be then back-projected to the three-dimensional coordinates of the mesh at block 3305e (in a prototype implementation, taking approximately 1.5 ms to complete) to produce the current estimated point-cloud representing the scene. In block 3305f the system may perform the cross-sectioning of the estimated point cloud, e.g., along the centerline, querying the centerline from the KD Tree from all of the estimated point cloud vertices and considering the previously generated flattened image 3305i (in a prototype implementation, taking approximately 75 ms to complete). Block 3305f may then produce the new flattened image 3305i for rendering.

FIG. 33B is a schematic block diagram various operational relations between components of a surface flattening image update process as may be implemented in some embodiments, e.g., at block 3305f. The centerline KD tree 3310d may be the output from block 3305h, point cloud 3320c may correspond to the back project point cloud from block 3305e, and the centerline 3320o may correspond to the centerline 3305g (corresponding to each input to block 3305f in FIG. 33A).

An index may be assigned to each vertex x of the currently constructed mesh, or of a back-projected point cloud from the estimated refined depth map (e.g., where the system performs this operation for only the “active” region around the camera), each index representing the closest point cₖ upon the centerline with K samples via the KD tree as shown in EQNs. 8 and 9:

input:X=x₁, . . . ,x_{N}∈R³, centerline KDTree (8)

{Sₖ}={arg min_{k∈{1, . . . ,K}}∥xᵢ−cₖ∥²} (9)

where {Sₖ} is the set representing all the vertices assigned to the kth point upon the centerline. Following this index assignment, each vertex may be assigned an angle encircling the centerline (e.g., representing the vertex's radial relation to the corresponding circumference). In some embodiments, the vertices may be grouped into sections in accordance with their angle assignment (e.g., based upon their presence in one of a collection of radial ranges), e.g., into groups of angle “bins” of approximately equal angle width (e.g., 0.5 degrees). In some embodiments, the system may employ an axis-angle representation where the axis-angle is the forward direction along the centerline (computed, e.g., between two adjacent samples along the centerline). As discussed, for each row in the projected image, the relevant columns (starting 0-359 degrees) may be colored in accordance with the assigned angle of the corresponding estimated point cloud vertex, e.g. as indicated by the “paint rows” block 3310l (in a prototype implementation, taking approximately 65 ms to complete). Thus, the cross section circumferences along the centerline points at block 3310i may be transformed to rows of the image at block 3310j and the updated pixels written at block 3310k (e.g., those pixels which have changed or are newly encountered) to produce an updated surface image 3310m. In some embodiments, rather than rewrite the entire image, the old pixels in the previous image 3310n may remain unchanged. Accordingly, the system may query the KD tree at block 3310e (in a prototype implementation, taking approximately 7.5 ms to complete), to determine a centerline 3310f and active row indices 3310g. The centerline 3305g may be used to extend the surface flattening image as discussed at block 3310h (in a prototype implementation, taking approximately Oms, i.e., a negligible amount, to complete).

With respect to the navigation compass, e.g., as discussed with respect to block 3215a, FIG. 33C is a schematic block diagram depicting various operational relations between components of a navigation compass update process 3315a as may be implemented in some embodiments. As mentioned, the compass (such as compass 2820) may be used to direct the user to un-inspected, or inadequately inspected, areas. The navigation compass may be linked to the surface parametrization block 3305a via the updated surface flattening image 3310m, which may be used to create the compass, e.g., by facilitating lacunae identification.

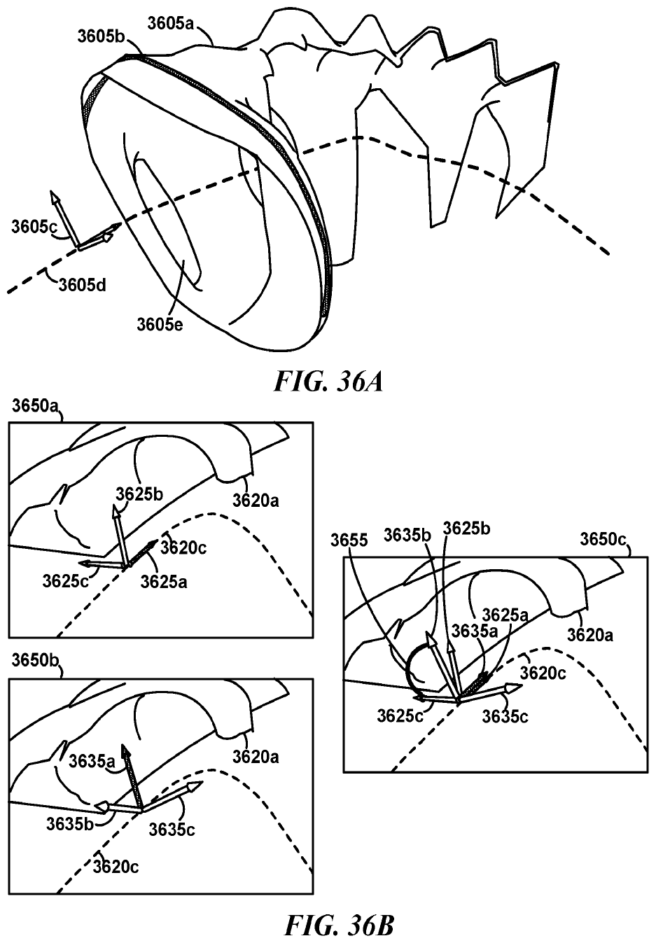

Process 3315a may receive as inputs the surface flattening image 3305i and the camera position 3315d. The overlap navigation assistance color block 3315h may use the current camera pose 3315i and corresponding portion of the flattened image 3315f to determine the appropriate radial coloring (e.g., where hues correspond to the consistent global radial degrees, as discussed, e.g., with respect to FIGS. 26C and 30B) before the camera pose and the 3D model coordinate system (defined by the centerline axis) are aligned at block 3315j (taking approximately 0.7 ms in some embodiments). After the alignment between the camera pose and the centerline forward vector (e.g., as discussed herein with respect to view 3650c of FIG. 36B) the offset angle between the x-axis may be computed in order to offset the compass visualization such that the navigation will be invariant to any camera roll (again, as discussed with respect to, e.g., FIG. 26C). Phrased differently for clarity, given a first “x-axis” vector vₓ₁ a first coordinate system as defined by the camera pose and a second “x-axis” vector vₓ₂ of a second coordinate system define by the centerline, the system may compute the angle using the axis-angle representation as indicated in EQN. 10:

$$angle = acos ( \frac{{v}_{x 1} \cdot {v}_{x 2}}{\vert {v}_{x 1} \vert \vert {v}_{x 2} \vert} ) \qquad ( 10 )$$

The navigation compass may then be created at block 3315k (in a prototype implementation, taking approximately 7 ms). Up-sampling for display may then occur at block 3315l (in a prototype implementation, taking approximately 2 ms to complete), before the navigation image 3315m is created, depicting the visual field image with the compass overlaid. The system may then combine this image 3315m with the surface flattening image 3315e and original image 3315g at block 3315n to be output to a display at block 3315b.

### Example Medial Axis Centerline Estimation—System Processes

Naturally, more precise and consistently generated centerlines may better enable more precise circumference selection for mapping. While one will appreciate a number of methods for dividing a model into circumferences (with or without the use of a centerline), this section provides example centerline estimation methods and processes for the reader's comprehension. Consistent centerline estimation, as may be achieved with these methods, may be particularly useful when analyzing and comparing surgical procedure performances. Accordingly, though presented with specific reference to the example of creating centerlines in the colonoscope context, various embodiments contemplate improved methods for determining the centerline based upon the localization and mapping process, e.g., as described previously herein with reference to FIG. 14, as applied in a variety of anatomies.

### Iterative Internal Body Structure Representation—Variations

One will appreciate variations of the embodiments described above. For example, in some embodiments, the vertical dimension of region 2535a is not fixed, but will grow as the examination continues. This may be appropriate where the dimensions of the organ are unknown. Given the finite space of a GUI, the user may be invited to scroll along the vertical dimension of region 2535a, or the vertical dimension may be scaled such that the available data always fits within the vertical dimension of region 2535a (e.g., there being no region 2540b corresponding to the open end of the mesh 2525a, but rather the available texture extended to the top of the region 2535a; magnified regions, like magnified region 2970b, may facilitate local review in such embodiments). However, in many surgical operations, at least the approximate dimensions of the interior region of the patient's body to be examined may be known. Accordingly. some embodiments may adjust region 2535a in accordance with these expectations. By doing so, the operator and other members of the surgical team may anticipate future states of the surgery and appreciate the present state and scope of review.

For example, similar to FIGS. 25 and 28, FIG. 34A and FIG. 34B depict successive schematic representations of various GUI panels as may be presented to a user during a surgical procedure in some embodiments. Here, the partially complete model 3405e shown in the views 3405b and 3405c may again include various lacunae with corresponding regions in the mapped representation 3405d (corresponding, e.g., to the partial models 2505a-c). As the camera advances, the field of view 3405a (corresponding to region 2510a) may be updated accordingly. One will appreciate that the two views 3405b and 3405c may be two simultaneously presented views of the same model from different orientations, or a single view rotated using, e.g., a mouse cursor 3405g. Unlike the embodiments of FIG. 25, however, the three-dimensional view includes a non-data derived reference mesh 3405f of the current model 3405e. The reference mesh 3405f may be determined by an average of other patients' organ models, a convex hull of their cumulative models (then scaled by the current patient's dimensions), an artist rendition of the organ, etc.

As the surgery progresses and more of the current patient's data is captured, the reference mesh may be replaced with corresponding portions of the data-created meshes 3405e and 3405h. Reference mesh 3405f may be rendered without texture or otherwise clearly distinguished from the data captured meshes 3405e and 3405h. By anticipating the full length of the organ, row placement from centerline circumferences within the finite vertical dimension of mapped representation 3405d may be managed accordingly. That is, if 30% of the reference mesh remains, then the captured data may be scaled so that approximately 30% of the vertical dimension in the region 3405d remains available and marked as “lacuna.” For example, in FIG. 34A, roughly 30% of the reference mesh 3405f remains and so the acquired data may be placed in region 3405d such that unexplored region 3490 comprises roughly 30% of the vertical dimension of region 3405d. Thus, corresponding to the expected length of the organ, the non-data derived mesh 3405f may anticipate the existence of regions in the surgical procedure yet to be explored. As shown in FIG. 34B, at a later time, the views 3405b, 3405c may replace the non-data derived mesh 3405f with the corresponding portions of the updated partial mesh 3405h.

In some embodiments, the non-data derived mesh 3405f may be an idealized geometric structure corresponding to the relevant anatomy. For example, as shown in FIG. 34C a cylindrical reference geometry mesh 3415a may be assumed to correspond 3415c to an actual data-derived intestinal mesh geometry 3415b. While the reference geometry mesh 3415a may be created by an artist by hand, one will appreciate that the dimensions may be determined by a variety of methods. For example, decimation of vertices on an mesh generated from averaged data-derived meshes may result in a near-cylindrical structure, which may itself be used, or an idealized cylinder with substantially similar radius and length. Thus, the idealized reference mesh may be generated based upon an accumulation of real-world data.

Similarly, while intestinal examination has been presented herein primarily to facilitate the reader's understanding, one will appreciate that many embodiments need not be limited to that context. For example, FIG. 34D and FIG. 34E depict perspective views of a reference spherical geometry mesh 3420a, a cumulative convex hull reference geometry mesh 3420b, and an example cavity mesh geometry 3425 captured from the current surgical procedure (e.g., a prostatectomy), respectively. As indicated, each of the refences meshes 3420a, 3420b may be used to correspond 3420c, 3420d to the mesh 3425 generated during the current surgical procedure. Just as iterative “consumption” of the reference mesh 3405f by the data meshes 3405e and 3405h facilitated a reference for determining overall progress, iterative consumption of meshes 3420a and 3420d during exploration of the cavity producing the mesh 3425 may likewise facilitate production of a mapped region, similar to region 3405d, depicting the relative overall progress during the surgery.

To determine what portions of a refence mesh should be replaced with portions of the data-derived mesh, the system may employ a process as shown in FIG. 34F. Here, the edges and vertices of the reference mesh are schematically presented by series of edges and vertices 3430a upon the two-dimensional plane of the page. For example, the edges and vertices 3430a could be the inner or outer surface of the cylinder 3415a, or the inner or outer surface of meshes 3420a or 3420b. As the data-derived mesh 3430b grows, the system may compare 3440 the meshes. Vertices within a threshold distance of a nearest neighbor vertex in the corresponding mesh may be construed as “associated” with the other mesh (represented here by arrows such as arrow 3445), while vertices outside the threshold remain unassociated. Finer granularity may be achieved in some situations by instead only taking the distance along a projection. The reference geometry may be adjusted throughout the process (e.g. rescaling and retargeting of mesh portions) as new data arrives to better ensure correspondence.

Here, the vertices 3435a and 3435b are within a threshold distance of at least one vertex in the mesh 3430b, at least when the difference vector between the vertices in three-dimensional space is projected along a centerline 3450c. For example the projected distance 3450a between the vertex 3435b and the vertex 3435d is less than the threshold. In contrast, the distance 3450b between the vertex 3435c and the vertex 3435d is greater than the threshold. As vertex 3435d is the nearest vertex in the mesh 3430b to vertex 3435d, vertices including and to the left of the vertex 3435b may be removed from the non-data-derived, reference mesh 3430a, while retaining the vertices including and to the right of vertex 3435c (i.e., remove the vertices in the reference mesh 3430a found to be associated with vertices in mesh 3430b).

### Graphical Supplements for Navigation and Orientation—Further Variations

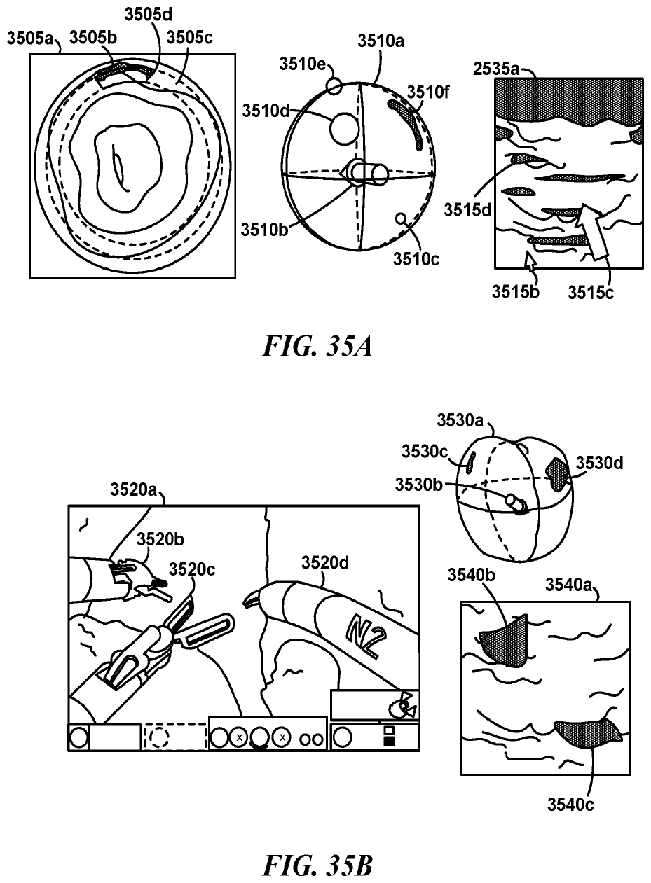

Additional variations upon the GUI features discussed above may be understood with reference to FIGS. 35A and 35B. Specifically, FIG. 35A is a schematic collection of GUI elements in an example colonoscope examination as may be presented to a reviewer in some embodiments. In one variation, as shown in view 3505a, the compass 3505c may assume a translucent form (represented here by dashed lines). A lacuna may be presented as a blob overlay 3505b in the user's field of view corresponding to the highlight 3505d appearing on the compass 3505c. For example, the blob overlay 3505b may be an augmented reality object, such as a billboard rendered upon the user's field of view. By rendering in front of the user' field of view, the blob overlay 3505b may be visible to the user, even if there are occluding objects (e.g., haustral folds) obscuring the lacuna from the camera's current field of view.

Similarly, in some embodiments, in lieu of, or complementary with, the compass 3505c, the system may present a three-dimensional compass 3510a to the user. In this example, the three-dimensional compass 3510a is a sphere with an arrow 3510b at its center indicating the direction from the camera's current orientation to a selected lacuna, or other regions of interest (e.g., the user may have selected the lacuna associated with region 3515d using a cursor 3515b). Just as highlights upon the compass 2820 indicated the location of lacuna in the camera's present vicinity, spheres (e.g., spheres 3510c, 3510d, 3510e) or other indicia may be placed upon locations in the spherical surface of compass 3510a to indicate locations relative to the camera. In some embodiments, for greater clarity, projections of the lacunae upon the compass' spherical surface, such as the lacuna projection 3510f, may also orient the user to the relative location and structure of a lacunae. Such three-dimensional representations, when combined with more two-dimensional representation (such as the current camera location indication 3515c) may empower the user with both quick and accurate navigational context under the time sensitive and high pressure conditions of surgery. Specifically, the user can generally assess their relative orientation by consulting the region 2535a. Once oriented, at the user's convenience, the user may then consult compass 2820, compass 3510a, or magnified region 2970b, etc. for a more granular assessment of their relative location.

As previously mentioned, one will readily appreciation application of various of the disclosed embodiments in surgical contexts other than colonoscopy. For example, FIG. 35B is a schematic collection of GUI elements in an example surgical robotic examination as may be presented to a reviewer in some embodiments. The user may be using a surgical robot to perform a prostatectomy and is operating within an inflated cavity of the patient's lower torso. Here, the user is presented with a camera view 3520a depicting the camera' field of view, which includes perspective views of the various instruments 3520b, 3520c, 3520d. One will appreciate that, like the partial models 2505a-c, progressive examination of the patient cavity may likewise produce a model 3530a with lacuna 3530c and 3530d. An orienting indicia, such as arrow 3530b, may indicate the present relative orientation of the surgical camera. Similarly the projected map 3540a may inform the user of the relative position of lacuna 3530c and 3530d via corresponding regions 3540b and 3540c. Here, one will appreciate that the unwrapped circumferences may be taken at various rotations about a central point of the cavity, rather than at points along the centerline as previously discussed.

### Example Prototype Graphical User Interfaces

FIG. 36A is a schematic representation of an incomplete model 3605a, contour circumference determination 3605b, and centerline guide path 3605d, in a prototype implementation of an embodiment. Here, the incomplete model 3605a produces a centerline 3605d from which circumferences, such as circumference 3605b, may be determined. Here, axes 3605c indicate a position and orientation of the camera at the time of data capture. As the camera proceeds forward, one will appreciate that the circumferences encountering the lacuna 3605e will result in a region of the projected map depicting a hole for 3605e.

Using such axes to facilitate the reader's comprehension, FIG. 36B is a collection of schematic perspective views 3650a, 3650b, 3650c of various orientation axes relative to a centerline during an example compass alignment process (e.g., to maintain alignment with a global reference as discussed with respect to FIG. 26C), as may be implemented in some embodiments. View 3650a depicts a schematic representation of an incomplete model 3620a, centerline guide path 3620c, and “centerline axes” 3625a-c. Specifically, the centerline vector 3625a indicates the forward direction (e.g., for advancing) along the centerline 3620c, the vectors 3625b and 3625c continuing to point in the same radial positions on the sidewall during the advance (e.g., corresponding to the same columns, such as 180 and 90 degrees, respectively, in the map 2535a; that is, the vector 3625b will track the 90 degree line of continuity 2555d and reference line 2630c as the axes 3625a-c move down the centerline 3620c).

In contrast, view 3650b depicts camera orientation axes 3635a-c, which remain consistent with the camera's orientation. Here, the forward vector 3635a indicates the direction in which the camera is pointing, vector 3635b the left direction (i.e., to the left in the field of view of the camera) and the vector 3635c the top direction (i.e., to the top in the field of view of the camera). To determine how to reorient the compass (e.g., as during rotation in FIG. 26C), some embodiments may align the axes 3635a-c to the axes 3625a-c by translating the axes 3635a-c to the same point upon the centerline as the axes 3625a-c and rotating vector 3635a to align with the forward centerline vector 3625a, as shown in the view 3650c (to facilitate the reader's comprehension the vectors 3635a and 3625a are not shown here being perfectly aligned, as they would be in practice). For clarity, rotating vector 3635a only (and not vectors 3635b and 3635c) to align with the vector 3625a, will still result in a change in orientation of the vectors 3635b and 3635c as shown. Once vector 3635a and 3625a are aligned, the angle 3655 between the left vector 3635b of the camera orientation axes 3635a-c and the left vector 3625b of the global forward centerline axes 3625a-c, may indicate the angle at which the compass is to be rotated in the field of view overlay, so as to remain in the same global orientation of the model (again, as was described with respect to FIG. 26C). The reader will appreciate that this demonstrates just one possible method for maintaining the compass' global orientation, e.g., at blocks 3315k or 3315m, so as to align the camera pose and the 3D model coordinate system.

### Computer System

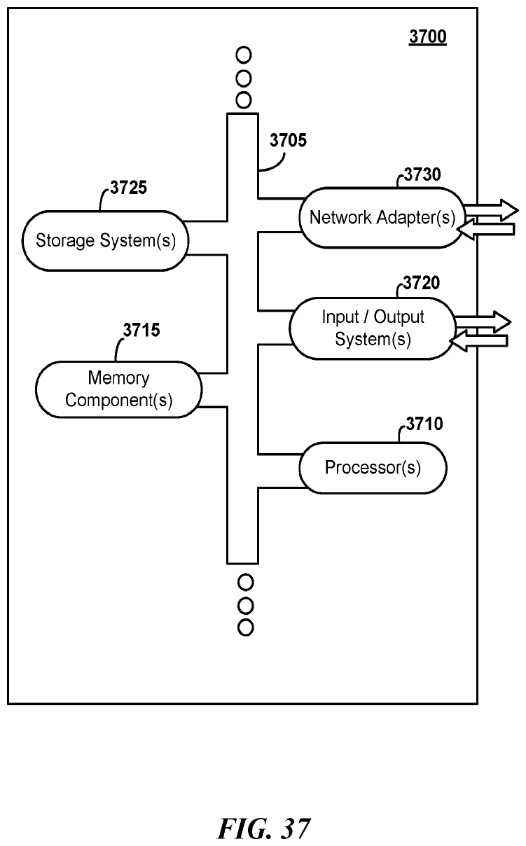

FIG. 37 is a block diagram of an example computer system as may be used in conjunction with some of the embodiments. The computing system 3700 may include an interconnect 3705, connecting several components, such as, e.g., one or more processors 3710, one or more memory components 3715, one or more input/output systems 3720, one or more storage systems 3725, one or more network adaptors 3730, etc. The interconnect 3705 may be, e.g., one or more bridges, traces, busses (e.g., an ISA, SCSI, PCI, 12C, Firewire bus, etc.), wires, adapters, or controllers.

The one or more processors 3710 may include, e.g., an Intel™ processor chip, a math coprocessor, a graphics processor, etc. The one or more memory components 3715 may include, e.g., a volatile memory (RAM, SRAM, DRAM, etc.), a non-volatile memory (EPROM, ROM, Flash memory, etc.), or similar devices. The one or more input/output devices 3720 may include, e.g., display devices, keyboards, pointing devices, touchscreen devices, etc. The one or more storage devices 3725 may include, e.g., cloud-based storages, removable Universal Serial Bus (USB) storage, disk drives, etc. In some systems memory components 3715 and storage devices 3725 may be the same components. Network adapters 3730 may include, e.g., wired network interfaces, wireless interfaces, Bluetooth™ adapters, line-of-sight interfaces, etc.

One will recognize that only some of the components, alternative components, or additional components than those depicted in FIG. 37 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 3730. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 3715 and one or more storage devices 3725 may be computer-readable storage media. In some embodiments, the one or more memory components 3715 or one or more storage devices 3725 may store instructions, which may perform or cause to be performed various of the operations discussed herein. In some embodiments, the instructions stored in memory 3715 can be implemented as software and/or firmware. These instructions may be used to perform operations on the one or more processors 3710 to carry out processes described herein. In some embodiments, such instructions may be provided to the one or more processors 3710 by downloading the instructions from another system, e.g., via network adapter 3730.

### REMARKS

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

1. A computer-implemented method for assessing surgical instrument progress within a patient interior, the method comprising:
determining pose data associated with a surgical instrument;
determining depth data associated with the pose data;
constructing at least a portion of a three-dimensional model of at least a portion of the patient interior based upon the pose data and the depth data; and
determining a position of a centerline associated with the at least the portion of the three-dimensional model.

2. The computer-implemented method of claim 1, wherein constructing the at least the portion of the three-dimensional model of the at least the portion of the patient interior based upon the pose data and the depth data, comprises:
determining features between two images;
generating a fragment based upon differences between the two features; and
consolidating the fragments to form the at least the portion of the three-dimensional model.

3. The computer-implemented method of claim 1, wherein determining the position of the centerline comprises:
providing the depth data to a neural network configured to in-fill portions of the at least the portion of the three-dimensional model.

4. The computer-implemented method of claim 1 further comprising:
determining a kinematics threshold, wherein, the kinematics threshold is one of:
a speed threshold for motion of at least a portion of the surgical instrument projected upon the centerline;
a speed threshold for motion of at least a portion of the surgical instrument projected radially from the centerline; and
a distance of at least a portion of the surgical instrument from the centerline.

5. The computer-implemented method of claim 4, wherein, the kinematics threshold is a speed threshold for motion of at least the portion of the surgical instrument projected upon the centerline, and wherein, the method further comprises:
determining that the surgical instrument is being withdrawn; and
determining that a speed of the surgical instrument projected upon the centerline exceeds the speed threshold.

6. The computer-implemented method of claim 5, wherein, determining the kinematics threshold comprises:
consulting a database comprising surgical instrument kinematics data projected upon reference geometries for a plurality of surgical operations; and
determining the kinematics threshold based upon kinematics data values in the database corresponding to times when surgical instruments were being withdrawn.

7. The computer-implemented method of claim 6, wherein determining the position of the centerline associated with the at least the portion of the three-dimensional model, comprises:
filtering the at least the portion of the three-dimensional model to produce a filtered portion;
determining centerline endpoints based upon the filtered portion;
generating a new local centerline from poses of the surgical instrument; and
extending the centerline with the new local centerline.

8. The computer-implemented method of claim 7, wherein extending the centerline with the new local centerline, comprises:
determining a first array of points on the centerline;
determining a second array of points on the new local centerline; and
determining a weighted average between pairs of points in the first array and in the second array.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A non-transitory computer-readable medium, the non-transitory computer-readable medium comprising instructions configured to cause a computer system to perform a method for assessing surgical instrument progress within a patient interior, the method comprising:
determining pose data associated with a surgical instrument;
determining depth data associated with the pose data;
constructing at least a portion of a three-dimensional model of at least a portion of the patient interior based upon the pose data and the depth data; and
determining a position of a centerline associated with the at least the portion of the three-dimensional model.

22. The non-transitory computer-readable medium of claim 21, wherein constructing the at least the portion of the three-dimensional model of the at least the portion of the patient interior based upon the pose data and the depth data, comprises:
determining features between two images;
generating a fragment based upon differences between the two features; and
consolidating the fragments to form the at least the portion of the three-dimensional model.

23. The non-transitory computer-readable medium of claim 21, wherein determining the position of the centerline comprises:
providing the depth data to a neural network configured to in-fill portions of the at least the portion of the three-dimensional model.

24. The non-transitory computer-readable medium of claim 21, wherein the method further comprises:
determining a kinematics threshold, wherein, the kinematics threshold is one of:
a speed threshold for motion of at least a portion of the surgical instrument projected upon the centerline;
a speed threshold for motion of at least a portion of the surgical instrument projected radially from the centerline; and
a distance of at least a portion of the surgical instrument from the centerline.

25. The non-transitory computer-readable medium of claim 24, wherein, the kinematics threshold is a speed threshold for motion of at least the portion of the surgical instrument projected upon the centerline, and wherein, the method further comprises:
determining that the surgical instrument is being withdrawn; and
determining that a speed of the surgical instrument projected upon the centerline exceeds the speed threshold.

26. The non-transitory computer-readable medium of claim 25, wherein, determining the kinematics threshold comprises:
consulting a database comprising surgical instrument kinematics data projected upon reference geometries for a plurality of surgical operations; and
determining the kinematics threshold based upon kinematics data values in the database corresponding to times when surgical instruments were being withdrawn.

27. The non-transitory computer-readable medium of claim 26, wherein determining the position of the centerline associated with the at least the portion of the three-dimensional model, comprises:
filtering the at least the portion of the three-dimensional model to produce a filtered portion;
determining centerline endpoints based upon the filtered portion;
generating a new local centerline from poses of the surgical instrument; and
extending the centerline with the new local centerline.

28. The non-transitory computer-readable medium of claim 27, wherein extending the centerline with the new local centerline, comprises:
determining a first array of points on the centerline;
determining a second array of points on the new local centerline; and
determining a weighted average between pairs of points in the first array and in the second array.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A computer system comprising:
at least one processor; and
at least one memory, the at least one memory comprising instructions configured to cause the computer system to perform a method for assessing surgical instrument progress within a patient interior, the method comprising:
determining pose data associated with a surgical instrument;
determining depth data associated with the pose data;
constructing at least a portion of a three-dimensional model of at least a portion of the patient interior based upon the pose data and the depth data; and
determining a position of a centerline associated with the at least the portion of the three-dimensional model.

42. The computer system of claim 41, wherein constructing the at least the portion of the three-dimensional model of the at least the portion of the patient interior based upon the pose data and the depth data, comprises:
determining features between two images;
generating a fragment based upon differences between the two features; and
consolidating the fragments to form the at least the portion of the three-dimensional model.

43. The computer system of claim 41, wherein determining the position of the centerline comprises:
providing the depth data to a neural network configured to in-fill portions of the at least the portion of the three-dimensional model.

44. The computer system of claim 41, wherein the method further comprises:
determining a kinematics threshold, wherein, the kinematics threshold is one of:
a speed threshold for motion of at least a portion of the surgical instrument projected upon the centerline;
a speed threshold for motion of at least a portion of the surgical instrument projected radially from the centerline; and
a distance of at least a portion of the surgical instrument from the centerline.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)
